SEQUENCE LISTING (6 sequences)

SEQ ID NO: 1 - protein, 462 aa, Cricetulus griseus
  REGION 1..462: note = misc_feature - Thioesterase (G3HNG5) glycan positions, 298, 422
MAQRLAPNIPEGFKAVTASLGRPRSTKAQPDSEAMQASTAQDQMAPIMILEPADGCLCDQPVLISVHGLAPEQPVTLRAALRDEKGALFRAHARYRADDHGGLDLARAPALGGSFAGIEPMGLLWALEPERPFWRLIKRDVQTPFVVELEVLDGHEPDGGRLLARAVHERHFMAPGVRRVPVREGRVRATLFLPPGNGPFPGIVDLFGVGGGLLEYRASLLAGKGFAVMALAYYNYDDLPKGMDIFHLEYFEEAVNYLLSHPQVKGPGIGLLGISKGGELGLAMASFLKGIKAAVIINGSVAAVGNTIHYKDETIPPVSLLRNRVKMTKDGLKDVVEGLQSPLVEEKSFIPVERPDTAFLLLVGQDDHNWKSEFYANEISKRLEAHGKEKPQIICYPEAGHYIEPPYFPLCKAGMHLLVGANITFGGEPKPHAVAQVDAWQQLQTFFHKHLGGEERTIPSKL

SEQ ID NO: 2 - protein, 412 aa, Homo sapiens
  REGION 1..412: note = misc_feature - LPLA2 (Q8NCC3, human sequence shown) glycan positions, 99, 273,289, 398
MGLHLRPYRVGLLPDGLLFLLLLLMLLADPALPAGRHPPVVLVPGDLGNQLEAKLDKPTVVHYLCSKKTESYFTIWLNLELLLPVIIDCWIDNIRLVYNKTSRATQFPDGVDVRVPGFGKTFSLEFLDPSKSSVGSYFHTMVESLVGWGYTRGEDVRGAPYDWRRAPNENGPYFLALREMIEEMYQLYGGPVVLVAHSMGNMYTLYFLQRQPQAWKDKYIRAFVSLGAPWGGVAKTLRVLASGDNNRIPVIGPLKIREQQRSAVSTSWLLPYNYTWSPEKVFVQTPTINYTLRDYRKFFQDIGFEDGWLMRQDTEGLVEATMPPGVQLHCLYGTGVPTPDSFYYESFPDRDPKICFGDGDGTVNLKSALQCQAWQSRQEHQVLLQELPGSEHIEMLANATTLAYLKRVLLGP

SEQ ID NO: 3 - protein, 548 aa, Cricetulus griseus
  REGION 1..548: note = misc_feature - PLBL2 (G3I6T1,listed without signal sequence) glycan positions,47, 65, 69, 190, 395, 474
LPTQGPGRRRQNLDPPVSRVRSVLLDAASGQLRLVDGIHPYAVAWANLTNAIRETGWAYLDLGTNGSYNDSLQAYAAGVVEASVSEELIYMHWMNTMVNYCGPFEYEVGYCEKLKSFLEINLEWMQREMELSQDSPYWHQVRLTLLQLKGLEDSYEGRLTFPTGRFTIKPLGFLLLQIAGDLEDLEQALNKTSTKLSLGSGSCSAIIKLLPGARDLLVAHNTWNSYQNMLRIIKKYQLQFRQGPQEAYPLIAGNNLVFSSYPGTIFSGDDFYILGSGLVTLETTIGNKNPALWKYVQPQGCVLEWIRNIVANRLALDGATWADIFKQFNSGTYNNQWMIVDYKAFIPNGPSPGSRVLTILEQIPGMVVVADKTEDLYKTTYWASYNIPFFEIVFNASGLQDLVAQYGDWFSYTKNPRAQIFQRDQSLVEDMNSMVRLIRYNNFLHDPLSLCEACIPKPNAENAISARSDLNPANGSYPFQALYQRPHGGIDVKVTSFSLAKRMSMLAASGPTWDQLPPFQWSLSPFRSMLHMGQPDLWTFSPISVPWD

SEQ ID NO: 4 - protein, 305 aa, Cricetulus griseus
  REGION 1..305: note = misc_feature - PPT (G3HN89) glycan positions, 197,212,232
MASPGSRWLLAVSLLPWCCAAWSLGHLNPPSLTPLVIWHGMGDSCCNPISMGAIKKMVEKEIPGIYVLSLEIGKNMMEDVENSFFLNVNSQVMMVCQILEKDPKLQQGYNAIGFSQGGQFLRAVAQRCPSPRMINLISVGGQHQGVFGLPRCPGESSHVCDFIRKMINAGAYSKVVQLRLVQAQYWHDPIKEDVYRNHSIFLADINQERCVNETYKKNLMALNKFVMVKFLNDSIVDPVDSEWFGFYRSGQAKETIPLQESTLYTEDRLGLKQMDKAGKLVFLAKEGDHLQLSKEWFNAYIIPFL

SEQ ID NO: 5 - protein, 488 aa, Cricetulus griseus
  REGION 1..488: note = misc_feature - PLD3 (G3HNQ5) glycan positions 97, 102, 132, 234, 282, 385, 430
MKPKLMYQELKVPVEEPAGELPVNEIEAWKAAEKKARWVLLVLILAVVGFGALMTQLFLWEYGDLHLFGPNQRPAPCYDPCEAVLVESIPEGLEFPNATTSNPSTSQAWLGLLAGAHSSLDIASFYWTLTNNDTHTQEPSAQQGEEILQQLQALAPRGVKVRIAVSKPNGPLADLQSLLQSGAQVRMVDMQKLTHGVLHTKFWVVDQTHFYLGSANMDWRSLTQVKELGVVMYNCSCLARDLTKIFEAYWFLGQAGSSIPSTWPRPFDTRYNQETPMEICLNGTPALAYLASAPPPLCPSGRTPDLKALLSVVDSARSFIYIAVMNYLPTMEFSHPRRFWPAIDDGLRRAAYERGVKVRLLVSCWGHSEPSMRSFLLSLAALRDNHTHSDIQVKLFVVPADEAQARIPYARVNHNKYMVTERAVYIGTSNWSGSYFTETAGTSLLVTQNGHDGLRSQLEDVFLRDWNSLYSHNLDTAADSVGNACRLL

SEQ ID NO: 6 - protein, 627 aa, Cricetulus griseus
  REGION 1..627: note = misc_feature - SP (A0A061IEQ5) glycan positions 84, 173, 333, 393, 518, 611
MPRHGVSSGQGHLRADLELRLESSLPAPKLGLLWMGLVLALVLTLFDSTVLWTPARAYPLPAQGHPVKFSAIVPPLQNAFVWRNLSCPACKVLFTALNYGLKKEPNVARVGSVAIKMCKMLNIAPLNVCQSAVHLFEDDVVEVWTRSVLSPSEACGLLLGPSCGHWDIFSSWNISLPSVPKPPPKPPSPPAPGAPVSRVLFLTDLHWDHDYLEGTDPNCADPLCCRRSSGWPPNSQAGAGYWGEYSKCDLPLRTLESLLKGLGPAGPFEMVYWTGDIPAHDVWQQSRQDQLRALTTVTDLVRKFLGPVPVYPAVGNHESTPVNGFPPPFIKGNQSSQWLYEAMAKAWEPWLPADALHTLRIGGFYALTPRPGLRLISLNMNFCSRENFWLLINSTDPAGQLQWLVEELQAAENRGDKVHIIGHIPPGHCLKSWSWNYYKIVARYENTLAGQFFGHTHVDEFEIFYDEETLSRPLAVAFLGPSATTYINLNPGYRVYQIDGNYPGSSHVVLDHETYILNLTQANAPEATPHWKRLYRARETYGLPDALPASWHNLVYRMRDNEQLFQTFWFLYHKGHPPSEPCGTPCRLATLCAQLSARADSPALCRHLMPNGSLPDAHSLWSRTLLC

(19) United States
(12) Patent Application Publication
(10) Pub. No.: US 2023/0048743 A1
(43) Pub. Date: Feb. 16, 2023

(54) Structures for Reducing Antibody-Lipase Binding

(71) Applicant: Genentech Inc., South San Francisco, CA (US)

(72) Inventors: Elizabeth Sara Hecht, South San Francisco, CA (US); Shrenik Chetan Mehta, South San Francisco, CA (US); Wendy Noel Sandoval, South San Francisco, CA (US); Sreedhara Alavattam, South San Francisco, CA (US); Nathaniel Robert Tzizik-Swanson, South San Francisco, CA (US)

(73) Assignee: Genentech Inc., South San Francisco, CA (US)

(21) Appl. No.: 17/811,316

(22) Filed: Jul. 8, 2022

Related U.S. Application Data
(60) Provisional application No. 63/319,686, filed on Mar. 14, 2022.
(60) Provisional application No. 63/231,134, filed on Aug. 9, 2021.
(60) Provisional application No. 63/220,894, filed on Jul. 12, 2021.

(51) Int. Cl. C07K 16/40; C12N 15/85

(52) U.S. Cl. CPC C07K 16/40; C12N 15/85; C07K 2317/522; C07K 2317/92; C07K 2317/524; C07K 2317/526; C07K 2317/41

(57) ABSTRACT

The present application relates to recombinant antibodies that are engineered to alter interactions between the antibodies and one or more endogenous lipases of a host cell used to produce the antibodies. In some cases, the antibodies are mutated in the heavy chain constant region, such as at CH1, CH2, and/or CH3. In other cases, the antibodies are mutated to alter their glycosylation profile.

### CROSS-REFERENCE TO RELATED APPLICATIONS

The present application claims priority to United States provisional patent application Nos: 63/319,686, filed Mar. 14, 2022, 63/231,134, filed Aug. 9, 2021, and 63/220,894, filed Jul. 12, 2021, each of which is incorporated by reference herein for any purpose.

### SEQUENCE LISTING

This application contains a Sequence listing in computer readable form entitled “01164-0020-00PCT”, created Jul. 5, 2022, having a size of 11000 bytes, which is incorporated by reference herein.

### FIELD

The present application relates to recombinant antibodies that are engineered to alter interactions between the antibodies and one or more endogenous lipases of a host cell used to produce the antibodies. In some cases, the antibodies are mutated in the heavy chain constant region, such as at CH1, CH2, and/or CH3. In other cases, the antibodies are mutated to alter their glycosylation profile.

### INTRODUCTION

The detection of Host cell proteins (HCPs) as contaminants in purified compositions of heterologous proteins expressed by the host cells has historically been challenging due to their low abundance. HCP impurities, for example, can lead to diverse product concerns and mechanistic characterization of their persistence and interactions with biotherapeutics is critical to refining processes. Lipases represent a class of HCPs that are believed to play a role in formulation shelf-life. Lipases, in the class of esterases, have the capacity to act on the excipient polysorbate 20 (PS-20), whose degradation can lead to sub-visible particles in a composition comprising a purified protein that also comprises polysorbate 20 as an excipient. While substantially depleted during downstream cellular processing, lipases at <0.1% levels, for example, may still deleteriously affect the stability of drug formulations. Efforts by surface plasmon resonance (SPR) have suggested that phospholipase B-like 2 (PLBL2) binds directly to antibodies, for example, but SPR did not detect certain other lipase-antibody complexes. For the first time, new methods using hydroxyl radical footprinting (FPOP), native mass spectrometry and ion mobility are used to directly establish, characterize and rank the interactions of multiple lipases and antibodies. Furthermore, the role of the lipase's and antibody's higher order structure is investigated for its impact on the binding affinity.

Here, FPOP was performed on control or lipase:antibody (Ab) molar ratio solutions to localize amino acids involved in binding. Antibody mutants were designed based on these predictions, and all proteins were expressed in CHO cell lines. N-glycan analysis of the lipases was performed on an HPLC-Chip Cube and PGC column on a Q-TOF (Agilent Technologies). Samples were exchanged on desalting spin columns just prior to mass spectrometry (MS) or ion mobility (IM) analysis. Static spray native MS and charge reducing non-MS IM quantitative assays were developed to screen complexes and analyzed on a Q Exactive™ UHMR (Thermo Fisher Inc.) or an IMgeniusrm (IonDX Inc.). Native MS deconvolution was performed in Unidec 3.1 with binding dissociation curves analyzed in R.

Targeted analysis was found to be critical to establishing the role of structure, sequence, and modifications in complex interactions. SEC and SPR caused disruption of complexes, leading to evaluation of lipases PLBL2 and lysosomal phospholipase A2 (LPLA2) in complex with several different monoclonal antibodies by microscale thermophoresis (MST). Non-immobilizing/labeling, solution-state native assays were desired to confirm the low affinities (1-30 μM Kd) and gain structural insights. FPOP localized conserved interactions to the Ab heavy chain, and accordingly, point mutations were produced. Low-resolving power native MS was used for detection to overcome high heterogeneity resulting from >5 glycosylation sites/lipase. Complexes were determined to have a 1:1 stoichiometry by MS (˜110 kDa) and by IM (˜51 inverse mobility (1/K) units). The rank order of antibody binding affinity was the same across methods. Both MS and 1M detected lipase-Ab interactions that were too weak for MST analysis (Lipase-C and D). PRM MS experiments were performed to generate binding dissociation curves as a function of HCD energy to rank antibodies and mutants. The relative area of the complex IM peak was also quantified and compared between species. Of the six mutants screened, one had near total binding disruption, with the others falling between 17-77% complex reduction. The trends in the rank orders of mutants were approximately the same, with the weakest and strongest knock outs consistent between methods. The IM data was further explored to gain insight into the protein confirmations adopted in the complex. Confirmed with time-course and exo-glycosidase experiments, shifts observed in the free lipase peak pre- and post-complex formation supported a secondary glycosylation-mediated mechanism for lipase-antibody interactions. In sum, this work is the first in-depth structural report of lipase-Ab binding, and also demonstrates the utility of orthogonal MS and newly established IM techniques in evaluating complexes.

### SUMMARY

In one embodiment, the invention includes an antibody (e.g., a recombinant antibody) produced by a cell (e.g., a mammalian host cell) engineered (e.g., transformed or transduced with a nucleic acid) to express the antibody, where the antibody has a modification (substitution, deletion, or addition) of at least 1 amino acid in the CH1 region, CH2 region, or CH3 region, and where the modification results in altered interaction of the antibody with one or more lipases (e.g., endogenous lipases) expressed by the cell. In one embodiment, the altered interaction is due to an altered glycosylation profile of said antibody. It is contemplated that any particular antibody may comprise one or more modifications, e.g., only a single modification, or modifications at two, three, four or more positions.

The invention also includes pharmaceutical compositions comprising any of the antibodies described herein, as well as isolated nucleic acid encoding such antibodies, expression vectors comprising such isolated nucleic acids, and cells containing, transformed or transduced with the isolated nucleic acids. Also included are methods of treating a human subject in need thereof, comprising administering to the subject a pharmaceutically effective dose of a pharmaceutical composition of the invention, as well as a method of producing the antibodies.

Thus, for example, the disclosure includes a recombinant antibody produced by a host cell engineered to express said antibody, wherein said antibody has a modification (substitution, deletion, or addition) of at least one amino acid residue in the heavy chain CH1 region, CH2 region, or CH3 region of the antibody, wherein the modification results in altered interaction of the antibody with one or more endogenous lipases expressed by said host cell. In some cases, the altered interaction is due to an altered glycosylation profile of said antibody. In some cases, the modification results in a reduced level of interaction with one or more endogenous lipases expressed by said host cell, such as lysosomal phospholipase A2 (LPLA2), phospholipase B-like protein (PLBL2), thioesterase, palmitoyl protein thioesterase (PPT), phospholipase D3 (PLD3), or sphingomyelin phosphodiesterase (SP). In some cases, the modification results in a reduced level of interaction with LPLA2 and/or PLBL2. In some cases, the modification results in at least a 5-fold, 10-fold, 20-fold, 30-fold, 50-fold, or 10)-fold reduction in binding affinity (i.e., increase in K_{D}) of said antibody to said one or more endogenous lipases expressed by said host cell, optionally wherein binding affinity is determined by surface plasmon resonance (SPR), microscale thermophoresis (MST), and/or ELISA. In some cases, the modification results in at least a 5-fold, 10-fold, 20-fold, 30-fold, 50-fold, or 100-fold reduction in the level of interaction of said antibody to said one or more endogenous lipases, optionally as determined by ESI-MS (e.g., by VC50) or by the amount of antibody-lipase complexes detected by SEC-MS or atmospheric ion mobility (e.g., IM-MS).

In some embodiments, the antibody comprises a human IgG constant region. In some cases, the antibody comprises a human IgG4 constant region, wherein the modification is a substitution of at least one amino acid from P149 to S197 (Kabat numbering). In some cases, the antibody comprises a human IgG1 constant region, wherein the modification is a substitution of an amino acid selected from V152 to P214 (Kabat numbering). In some cases modification comprises a substitution of at least one amino acid selected from the group consisting of G170, V171.1173, F174, P175, V177. L178, Q179. S180, S181, G182, L186, F154, P155, V189, V190, T191, V192, P193, S194, S195, S196, L198, K200, P157, V158, and T159 (Kabat numbering). In some cases, the modification comprises a substitution of an amino acid selected from the group consisting of F174, P175, Q179, V192, L198 and K200 (Kabat numbering). In some cases, the substitution is selected from the group consisting of G170A, V171A, T173A, F174A, P175A, V177A, L178A, Q179A, S180A, S181A, G182A, L186A, F154A, P155A, V189A, V190A, T191A, V192A, P193A, S194A, S195A, S196A, L198A, K200A, P157A, V158A, and T159A (Kabat numbering). In some cases, the substitution is selected from the group consisting of F174A, P175A, Q179A, V192A, L198A and K200A.

Alternatively, in some cases, the at least one substitution of at least one of G170, V171, T173, F174, P175, V177, L178, Q179, S180, S181, G182, L186, F154, P155, V189, V190, T191, V192, P193, S194, S195, S196, L198, K200, P157, V158, and T159 is with an amino acid selected from the group consisting of alanine (A), leucine (L) and isoleucine (I). In some cases, the at least one substitution is with alanine (A). In other cases, the at least one substitution is with an amino acid selected from the group consisting of phenylalanine (F), tryptophan (W) and tyrosine (Y). In some cases, the at least one substitution is with tryptophan (W), and in other cases the at least one substitution is with tyrosine (Y). In some cases the at least one substitution at one or more of G170, V171, T173, F174, P175, V177, L178, Q179, S180, S181, G182, L186, F154, P155, V189, V190, T191, V192, P193, S194, S195, S196, L198, K200, P157, V158, and T159 is with an amino acid selected from the group consisting of aspartic acid (D) and glutamic acid (E). In other cases, the at least one substitution is with an amino acid selected from the group consisting of Arginine (R) and Lysine (K).

In some embodiments, the antibody comprises modifications in one amino acid in the CH1 region. In some cases, the antibody comprises modifications in two amino acids in the CH1 region. In some cases, the antibody comprises modifications in three amino acids in the CH1 region. In some cases, the antibody comprises modifications in four or more amino acids in the CH1 region. In some cases, in addition to the modifications above, the antibody comprises at least one further modification in the heavy chain constant region, such as a modification in the Fc region, a mutation at N297, a LALAPG modification of an IgG1 Fc, and a substitution at one or more of residues 265, 269, 270, 297, 327, 333, 334, and 335 (EU numbering).

In some cases, the antibody heavy chain does not comprise a modification in an amino acid selected from residues 203-256 (Kabat numbering), does not comprise a modification in an amino acid selected from residues 203-243 (Kabat numbering), or does not comprise a modification of an amino acid selected from residues 197 and 198 and 203-243, and 246-251 (Kabat numbering). In some cases, the antibody is a human IgG4 antibody, and does not comprise a modification in an amino acid selected from any one or more of S197, L198, K203, T207, D211, R222, E226, S227, L229, G230, P237, P238, E246, F247, G249, G250, or P251. In some cases, the host cell is a Chinese hamster ovary (CHO) cell. In some cases, the host cell is modified to: mutate, down-regulate, or knock-out one or both of alpha-Man-1 or alpha-Man-2; to inhibit processing of Asn-linked Man₉GlcNac₂ glycan precursors and/or to increase high molecular weight mannose species, such as Man6 or higher, Man7 or higher, or Man7-9, relative to Man3-5; and/or to increase expression of one or more enzymes that increase the chain length of glycans, such as GNT-1, GNT-2, GNT-3, GNT-4abc, GNT-5, or GalT.

The present disclosure also relates to pharmaceutical compositions comprising antibodies described above. The disclosure further relates to a method of treating a human subject in need thereof, comprising administering to said subject a pharmaceutically effective dose of the pharmaceutical composition. The disclosure also relates to isolated nucleic acids expressing such antibodies, or a set of nucleic acids expressing the heavy and light chains of the antibodies, expression vectors comprising the nucleic acids, and isolated host cells containing, transformed or transduced with the isolated nucleic acids or expression vectors. The disclosure further relates to methods of producing the antibodies, comprising incubating a host cell containing, transformed or transduced with an isolated nucleic acid that expresses the antibodies under conditions in which the antibodies are produced.

The present disclosure also relates to methods of reducing interactions between a recombinant antibody and one or more endogenous lipases expressed in a host cell used to express the antibody, comprising engineering a modification (substitution, deletion, or addition) of at least one amino acid residue in the heavy chain CH1 region, CH2 region, or CH3 region of the antibody. In some cases, the method further comprises detecting interaction between the antibody and the one or more endogenous lipases or determining the binding affinity of the one or more endogenous lipases to the antibody. In some cases, the interaction of lipase and antibody is detected in an assay using purified lipase and antibody. In some cases, the interaction of lipase and antibody is detected by analysis of antibody produced in the host cell, for example by SPR, hydroxyl radical footprinting, native mass spectrometry (e.g., ESI-MS or SEC-MS), and/or ion mobility. In some cases, the method further comprises determining binding affinity of lipase and antibody, for example by surface plasmon resonance (SPR), microscale thermophoresis (MST), and/or ELISA.

In some such methods, the antibody modification results in a reduced level of interaction with one or more endogenous lipases expressed by said host cell, such as lysosomal phospholipase A2 (LPLA2), phospholipase B-like protein (PLBL2), thioesterase, palmitoyl protein thioesterase (PPT), phospholipase D3 (PLD3), or sphingomyelin phosphodiesterase (SP). In some cases, the modification results in a reduced level of interaction with LPLA2 and/or PLBL2. In some cases, the modification results in at least a 5-fold, 10-fold, 20-fold, 30-fold, 50-fold, or 100-fold reduction in binding affinity (i.e., increase in K_{D}) of said antibody to said one or more endogenous lipases expressed by said host cell, optionally wherein binding affinity is determined by surface plasmon resonance (SPR), microscale thermophoresis (MST), and/or ELISA. In some cases, the modification results in at least a 5-fold, 10-fold, 20-fold, 30-fold, 50-fold, or 100-fold reduction in the level of interaction of said antibody to said one or more endogenous lipases, optionally as determined by ESI-MS (e.g., by VC50) or by the amount of antibody-lipase complexes detected by SEC-MS or atmospheric ion mobility (e.g., IM-MS).

In some such methods, the antibody comprises a human IgG constant region. In some cases, the antibody comprises a human IgG4 constant region, wherein the modification is a substitution of at least one amino acid from P149 to S197 (Kabat numbering). In some cases, the antibody comprises a human IgG1 constant region, wherein the modification is a substitution of an amino acid selected from V152 to P214 (Kabat numbering). In some cases modification comprises a substitution of at least one amino acid selected from the group consisting of G170, V171, T173, F174, P175, V177, L178, Q179, S180, S181. G182, L186, F154, P155, V189, V190, T191, V192, P193, S194, S195, S196, L198, K200, P157. V158, and T159 (Kabat numbering). In some cases, the modification comprises a substitution of an amino acid selected from the group consisting of F174, P175, Q179, V192, L198 and K200 (Kabat numbering). In some cases, the substitution is selected from the group consisting of G170A, V171A, T173A, F174A, P175A, V177A, L178A, Q179A, S180A, S181A, 0182A, L186A, F154A, P155A, V189A, V190A, T191A, V192A, P193A, S194A, S195A, S196A, L198A, K200A, P157A, V158A, and T159A (Kabat numbering). In some cases, the substitution is selected from the group consisting of F174A, P175A, Q179A, V192A, L198A and K200A.

Alternatively, in some of these methods, the at least one substitution of at least one of G170, V171, T173, F174, P175, V177, L178, Q179, S180, S181, G182, L186, F154, P155, V189, V190, T191, V192, P193, S194, 5195, S196, L198, K200, P157, V158, and T159 is with an amino acid selected from the group consisting of alanine (A), leucine (L) and isoleucine (I). In some cases, the at least one substitution is with alanine (A). In other cases, the at least one substitution is with an amino acid selected from the group consisting of phenylalanine (F), tryptophan (W) and tyrosine (Y). In some cases, the at least one substitution is with tryptophan (W), and in other cases the at least one substitution is with tyrosine (Y). In some cases, the at least one substitution at one or more of G170, V171, T173, F174, P175, V177, L178, Q179, S180, S181, 0182, L186, F154, P155, V189, V190, T191, V192, P193, S194, S195, S196, L198, K200, P157, V158, and T159 is with an amino acid selected from the group consisting of aspartic acid (D) and glutamic acid (E). In other cases, the at least one substitution is with an amino acid selected from the group consisting of Arginine (R) and Ly sine (K).

In some of the methods, the antibody comprises modifications in one amino acid in the CH1 region. In some cases, the antibody comprises modifications in two amino acids in the CH1 region. In some cases, the antibody comprises modifications in three amino acids in the CH1 region. In some cases, the antibody comprises modifications in four or more amino acids in the CH1 region. In some cases, in addition to the modifications above, the antibody comprises at least one further modification in the heavy chain constant region, such as a modification in the Fc region, a mutation at N297, a LALAPG modification of an IgG1 Fe, and a substitution at one or more of residues 265, 269, 270, 297, 327, 333, 334, and 335 (EU numbering).

In some such methods, the antibody heavy chain does not comprise a modification in an amino acid selected from residues 203-256 (Kabat numbering), does not comprise a modification in an amino acid selected from residues 203-243 (Kabat numbering), or does not comprise a modification of an amino acid selected from residues 197 and 198 and 203-243, and 246-251 (Kabat numbering). In some cases, the antibody is a human IgG4 antibody, and does not comprise a modification in an amino acid selected from any one or more of S197, L198, K203, T207, D211, R222, E226, S227, L229, O230, P237, P238, E246, F247, G249, G250, or P251. In some cases, the host cell is a Chinese hamster ovary (CHO) cell. In some cases, the host cell is modified to: mutate, down-regulate, or knock-out one or both of alpha-Man-1 or alpha-Man-2; to inhibit processing of Asn-linked Man9GlcNac2 glycan precursors and/or to increase high molecular weight mannose species, such as Man6 or higher, Man7 or higher, or Man7-9, relative to Man3-5; and/or to increase expression of one or more enzymes that increase the chain length of glycans, such as GNT-1, GNT-2, GNT-3, GNT-4abc, GNT-5, or GalT.

The disclosure also contemplates methods of producing a recombinant protein or antibody, comprising: (a) expressing the protein or antibody in a host cell modified to: mutate, down-regulate, or knock-out one or both of alpha-Man-1 or alpha-Man-2; inhibit processing of Asn-linked Man9GlcNac2 glycan precursors and/or to increase high molecular weight mannose species, such as Man6 or higher, Man7 or higher, or Man7-9, relative to Man3-5; and/or increase expression of one or more enzymes that increase the chain length of glycans, such as GNT-1, GNT-2, GNT-3, GNT-4abc, GNT-5, or GaIT; and (b) determining whether the protein or antibody has reduced interaction with one or more endogenous lipases expressed by said host cell as compared to an antibody expressed from an unmodified host cell. In such methods, in some cases the host cell is a CHO cell. In some cases, determining whether the protein or antibody has reduced interaction with one or more endogenous lipases is by SPR, hydroxyl radical footprinting, native mass spectrometry (e.g., ESI-MS or SEC-MS), and/or ion mobility analysis of the protein or antibody expressed from the host cell as compared to the antibody expressed from an unmodified host cell.

The disclosure further encompasses methods of producing a recombinant protein or antibody, comprising: (a) expressing the protein or antibody in a host cell under conditions that significantly increase the concentration of high molecular weight mannose species, such as Man6 or higher, Man7 or higher, or Man7-9 relative to overall mannosylated species, and (b) determining whether the protein or antibody has reduced interaction with one or more endogenous lipases expressed by said host cell as compared to an antibody expressed from an unmodified host cell. In some such cases, the conditions comprise increasing the osmolality of the culture medium, for example, by at least 100 or at least 200 mOsm/kg, adding manganese chloride or ammonium chloride to the medium, increasing or adding raffinose, monensin, mannose, galactose, fructose, and/or maltose, or adding a high mannose promoting inhibitors such as Kifunensine to the medium. In such methods, in some cases the host cell is a CHO cell. In some cases, determining whether the protein or antibody has reduced interaction with one or more endogenous lipases is by SPR, hydroxyl radical footprinting, native mass spectrometry (e.g., ESI-MS or SEC-MS), and/or ion mobility analysis of the protein or antibody expressed from the host cell as compared to the antibody expressed from an unmodified host cell.

The disclosure also relates to methods of producing a recombinant protein or antibody, comprising: (a) expressing the protein or antibody in a host cell modified to eliminate at least one glycosylation site on at least one endogenous lipase; and (b) determining whether the protein or antibody has reduced interaction with one or more endogenous lipases expressed by said host cell as compared to an antibody expressed from an unmodified host cell. In some cases, the host cell is modified to eliminate at least one glycosylation site on LPLA2 and/or PLBL2. In some cases, the modification comprises at least one amino acid substitution within at least one N-X-S/T site in at least one lipase enzyme of the host cell. In such methods, in some cases the host cell is a CHO cell. In some cases, determining whether the protein or antibody has reduced interaction with one or more endogenous lipases is by SPR, hydroxyl radical footprinting, native mass spectrometry (e.g., ESI-MS or SEC-MS), and/or ion mobility analysis of the protein or antibody expressed from the host cell (e.g., 1M-MS) as compared to the antibody expressed from an unmodified host cell.

In some cases, where the host cell is a CHO cell comprising a modified lipase, the modification comprises:

one or more amino acid substitutions in LPLA2 (SEQ ID NO: 2) at one or more of positions 39-41, 99-101, 273-275, and 289-291,one or more amino acid substitutions in LPLA2 (SEQ ID NO: 2) at one or more of positions 125-131, 133-145, 146-177, 229-247, and 248-260

one or more amino acid substitutions in LPLA2 (SEQ ID NO: 2) at one or more of positions 146-177,

one or more amino acid substitutions in PLBL2 (SEQ ID NO: 3) at one or more of positions 47, 65, 69, 190, 395, and 474,

one or more amino acid substitutions in PLBL2 (SEQ ID NO: 3) at one or more of positions 67-78, 79-98, 173-187, 359-371, 372-388, 389-400, 401407, 424-459, 211-236, 241-253, 287-333, 340-352, 513-530, 539-546, 573-599, 56-64, 485-512, and 548-572,

one or more amino acid substitutions in PLBL2 (SEQ ID NO: 3) at one or more of positions 79-98, 424-459, 573-599, 372-388, and 548-572,

one or more amino acid substitutions in thioesterase (SEQ ID NO: 1) at one or more of positions 298-300 and 422-424,

one or more amino acid substitutions in PPT (SEQ ID NO: 4) at one or more of positions 197-199, 212-214, and 232-234,

one or more amino acid substitutions in PLD3 (SEQ ID NO: 5) at one or more of positions 97-99, 102-104, 132-134, 234-236, 282-284, 385-387, and 430-432, and/or

one or more amino acid substitutions in SP (SEQ ID NO: 6) at one or more of positions 84-86, 173-175, 333-335, 393-395, 518-520, and 611-613.

### BRIEF DESCRIPTION OF THE DRAWINGS

FIGS. 1A-1I show quality control mass spectra of: FIG. 1A, LPLA2-01 (LPLA Lot 1), FIG. 1B, LPLA2-02 (LPLA2 Lot 2), FIG. 1C, LPLA2-03, FIG. 1D, PLBL2-01, FIG. 1E, PLBL2-02, and overlays of the deconvoluted masses from the 1-3 production batches of: FIG. 1F, LPLA2, and FIG. 1G, PLBL2. FIG. 1H shows the deglycosylated mass of LPLA2 in the LPLA2-01 (LPLA2 reference) and LPLA2-02 (LPLA2 lot 2) samples. It shows the raw data from the RPLC-MS experiment. FIG. 1I shows the deglycosylated mass of PLBL2-01 (“reference”: shown in upper graphs) and PLBL2-02 (“lot 2” and lower graphs) samples.

FIGS. 2A-2C show quality control mass spectra of: FIG. 2A, antibody mAb1 (an IgG4 isotype antibody), FIG. 2B, mAb2, and FIG. 2C, mAb3.

FIG. 3A-3C show native mass spectra of 10:1 lipase to mAb1 binding to LPLA2-01 (FIG. 3A), PLBL2-01 (FIG. 3B), and PLBL2-02 (FIG. 3C). The pressure in the HCD cell was higher to stabilize the LPLA2 complex, resulting in increased antibody dimer artifacts in the spectrum.

FIGS. 4A and 4B show LPLA2-01-mAb1 complex formation at an (FIG. 4A) 10:1 molar ratio and (FIG. 4B) 1:10 molar ratio. The antibody peak remains the most intense species regardless of the solution ratio. However, the reduced concentrations at the 1:10 molar ratio in FIG. 4B prevent significant overlapping distributions from the Ab and Ab dimer.

FIG. 5A-SC show that ion mobility spectra of mAb2 (an IgG1 isotype antibody; FIG. 5A), mAb3 (an IgG1 isotype antibody FIG. 5B), and mAb1 (an IgG4-B isotype antibody; FIG. 5C) complexed to PLBL2-01 resulted in different quantities of 1:1 complex detected at approximately 52 l/K.

FIG. 6 shows that desialylated LPLA2 bound to mAb1 (IgG4 isotype) with similar affinity as detected by native MS.

FIG. 7 shows annotation of de-sialylated LPLA2 or glycoform analysis.

FIG. 8A-SC show relative abundance of sialylated, mannosylated, and fucosylated N-gly can species in each lipase indicated (LPLA2-02 in FIG. 8A, PLBL2-02 in FIG. 8B, and PLBL2-03 in FIG. 8C) in its active, TO form and inactive T6mo, form.

FIG. 9A-B show a comparison of mannosylated (FIG. 9A) or fucosylated (FIG. 9B) glycans between LPLA2-02, PLBL2-02, and PLBL2-03.

FIGS. 10A-C show: FIG. 10A, change in oxidation levels of LPLA2 peptides with and without mAb1 (IgG4 isotype), FIG. 10B, change in oxidation levels of LPLA2 peptides with and without mAb2 (IgG1 isotype), and FIG. 10C, binding epitopes of mAb1 and mAb2 mapped to the structure of LPLA2.

FIGS. 11A-C show: FIG. 11A, change in oxidation levels of PLBL2 peptides with and without mAb1 (IgG4 isotype), FIG. 11B, change in oxidation levels of PLBL2 peptides with and without mAb2 (IgG1 isotype), and FIG. 11C, binding epitopes of mAb1 and mAb2 mapped to the structure of PLBL2.

FIGS. 12A-B show changes in oxidation levels of peptides in mAb1 with and without either LPLA2 (FIG. 12A) or PLBL2 (FIG. 12B).

FIGS. 13A-B show changes in oxidation levels of peptides in mAb2 with and without either LPLA2 (FIG. 13A) or PLBL2 (FIG. 13B).

FIGS. 14A-B show low resolving power (4375 RP@200 m/z) of PLBL2 (FIG. 14A) and LPLA2-03 (FIG. 14B) binding to mAb1 mutants with alanine substitutions at positions 174, 175, 179, 192, 198, and 200, respectively. All intensity is relative to the base peak of the maximum charge state of the free antibody. The +21 peak of mAb1, at about 7095 m/z and the +20 peak at 7450 m/z overlap the complex distributions, but may be distinguished on the basis of exact mass, and by the differences in peak splitting, where the lipase glycoforms produce very wide spectral peaks. A 20 m/z window was used to isolate the +29 charge state of mutant L198A-LPLA2-03 complex, the +29 peak could be observed by SIM, but the complex distribution was not above the noise in the full spectrum.

FIGS. 15A-B show binding dissociation curves for (FIG. 15A) LPLA2-03-mAb1 mutant complexes or (FIG. 15B) PLBL2-03-mAb1 mutant complexes using a four-parameter logistic growth curve, with outliers detected by the Tukey method removed from calculations (highlighted in red). mAb1 mutants are, respectively F174A, P175A, Q179A, V192A, L198A, K200A. Unmutated mAb1 (WT) was also assessed.

FIG. 16A-16B show VC50 values extracted from native MS binding dissociation curves. Confidence intervals are shown in the error bars, with significant differences to WO at 95% or 90% confidence levels shown as * or +, respectively. FIG. 16k PLBL2 complex; FIG. 16B: LPLA2 complex FIG. 17A-17G show ion mobility analysis of mutant mAb1 species (FIGS. 17A-F) or wild-type (WT) mAb1 (FIG. 17G) against LPLA2-03. mAb1 mutants are, respectively F174A (FIG. 17A), P175A (FIG. 17B), Q179A (FIG. 17C), V192A (FIG. 17D), L198A (FIG. 17E), K200A (FIG. 17F). Fit curves are labeled with the peak assignment, where LPLA2 is the remaining free lipase, the Ab is the remaining free Ab, 2×Ab is a gas-phase dimer artifact, 2×LPLA2 is a gas-phase dimer artifact, and complex is the peak of interest. The fit sum is the curve created from the overlay of the Gaussian-A plots, and the thick lines plotted show the mean+/−standard deviation at east inverse mobility. Fits were performed in MagicPlot Pro® as described in the Examples.

FIGS. 18A-B show (FIG. 18A) 1:1 PPT:mAb1 stoichiometric complex formed in a solution at 100:1 relative molar concentrations and (FIG. 18B) 2:1 PLD3:mAb1 complex formed at 10:1 relative molar concentrations in solution. PLD3 was found naturally in solution as a dimer. The mAb peak at 6439 m/z (+23) is set to 100% relative abundance in each spectrum for scale. The antibody concentration is 0.27 μM.

FIGS. 19A-B show (FIG. 19A) relative abundance of sialylated, mannosylated, and fucosylated N-glycan species in each lipase, and (FIG. 19B) the breakdown in the mannose species between lipases. For glycans containing both a fucose and sialic species, its abundance in FIG. 19A was counted as contributing to both the fucosylated and sialylated groups.

FIG. 20 shows a schematic of the non-MS atmospheric ion mobility technology for detecting native state proteins and complexes. Samples flow through an electrospray emitter at 300 nL/min producing charged droplets, which pass through a charge reducing field. Droplets emerge with a single charge and evaporate yielding singly charged ions. At a given voltage, proteins of different collisional cross sectional areas take different trajectories around a central rod, where only ions with a certain inverse mobility will hit and be detected on the ring. A sweep of the voltage enables the relative proportion of sample components to be identified in a single two-minute run.

### DETAILED DESCRIPTION

### Definitions

As utilized in accordance with the present disclosure, the following terms, unless otherwise indicated, shall be understood to have the following meanings:

The term “antibody” herein refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term is used in the broadest sense and encompasses various antibody structures, including but not limited to monoclonal antibodies, polyclonal antibodies, multispecific antibodies (e.g., bispecific antibodies, diabodies, etc.), full length antibodies, single-chain antibodies, antibody conjugates, and antibody fragments, so long as they exhibit the desired binding activity.

An “isolated” antibody is one that has been separated from a component of its natural environment. In some aspects, an antibody is purified to greater than 95°,o or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

A “recombinant antibody” is an antibody that is produced in a host cell from a heterologous nucleic acid that has been introduced to the host cell for the purpose of producing the antibody, such as, for example, a vector.

An “antigen” refers to the target of an antibody, i.e., the molecule to which the antibody specifically binds. The term “epitope” denotes the site on an antigen, either proteinaceous or non-proteinaceous, to which an antibody binds. Epitopes on a protein can be formed both from contiguous amino acid stretches (linear epitope) or comprise non-contiguous amino acids (conformational epitope), e.g., coming in spatial proximity due to the folding of the antigen. i.e. by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents.

“Affinity” or “binding affinity” refers to the strength of the sum total of noncovalent interactions between a single binding site of a molecule (e.g., an antibody) and its binding partner (e.g., an antigen or a lipase protein). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein.

In this disclosure. “binds” or “binding” or “specific binding” and similar terms, when referring to a protein and its ligand or an antibody and its antigen target for example, means that the binding affinity is sufficiently strong that the interaction between the members of the binding pair cannot be due to random molecular associations (i.e. “nonspecific binding”). Such binding typically requires a dissociation constant (K_{D}) of 1 μM or less, and may often involve a K_{D} of 100 nM or less.

In some cases, binding is detected using mass spectroscopy, such as by ion mobility and native mass spectroscopy (IM-MS). In some embodiments, the level of binding may be evaluated by determining the collisional-induced dissociation voltage “VC50” of a complex. A “VC50” is the voltage required to dissociate 50% of a given complex observed in the MS.

The term “heavy chain” refers to a polypeptide comprising at least a heavy chain variable region, with or without a leader sequence. In some embodiments, a heavy chain comprises at least a portion of a heavy chain constant region, such as at least a CH1 region, for instance. The term “full-length heavy chain” refers to a polypeptide comprising a heavy chain variable region and a complete heavy chain constant region, with or without a leader sequence. The full-length heavy chain of an IgG isotype antibody may or may not comprise a C-terminal lysine residue or C-terminal glycine-lysine residues.

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence.

The terms “full-length antibody”, “intact antibody”, and “whole antibody” are used herein interchangeably to refer to an antibody having a structure substantially similar to a native antibody structure or, comprising a full-length heavy chain (i.e., a complete Fc sequence) and a full-length light chain.

The term “hypervariable region” or “HVR” as used herein refers to each of the regions of an antibody variable region which are hypervariable in sequence and which determine antigen binding specificity, for example “complementarity determining regions” (“CDRs”). Generally, antibodies comprise six CDRs: three in the VH (CDR-H1 or heavy chain CDR1, CDR-H2, CDR-H3), and three in the VL (CDR-L1, CDR-L2, CDR-L3). Exemplary CDRs herein include:

(a) “Chothia CDRs”: hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) “Kabat CDRs”: CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and

(c) “McCallum CDRs”: antigen contacts occurring at amino acid residues 27c-36 (L1), 46-55 (L2), 89-96 (L3). 30-35b (H1), 47-58 (H2), and 93-101 (H3) (MacCallum et al. J. Mol. Biol. 262: 732-745 (1996)).

“Framework” or “FR” refers to the residues of the variable region residues that are not part of the complementary determining regions (CDRs). The FR of a variable region generally consists of four FRs: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4. An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

The term “variable region” or “variable domain” interchangeably refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). See, e.g., Kindt et al. Kuby Immunology, 6^{th} ed., W.H. Freeman and Co., page 91 (2007). A variable domain may comprise heavy chain (HC) CDR1-FR2-CDR2-FR3-CDR3 with or without all or a portion of FR1 and/or FR4: and light chain (LC) CDR1-FR2-CDR2-FR3-CDR3 with or without all or a portion of FR1 and/or FR4. That is, a variable domain may lack a portion of FR1 and/or FR4 so long as it retains antigen-binding activity. A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The light chain and heavy chain “constant regions” of an antibody refer to additional sequence portions outside of the FRs and CDRs and variable regions. Certain antibody fragments may lack all or some of the constant regions. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “Fc region” herein is used to define a C-terminal region of an immunoglobulin heavy chain that comprises at least the CH2 and CH3 portion of the constant region. The term includes native sequence Fc regions and variant Fc regions. Recombinant antibodies, produced by host cells, may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine and lysine. Therefore, in a recombinant antibody with a full-length heavy chain, the C-terminal lysine, or the C-terminal glycine and lysine, of the Fc region may or may not be present. Unless otherwise specified herein, numbering of amino acid residues in the Fc region or heavy chain constant region is according to Kabat numbering.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the human IgG1 IgG2, IgG3, or IgG4 isotype. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson. Nature Biotechnology 23:1126-1136 (2005).

The term “multispecific” herein refers to a molecule that can bind to more than one different target or antigen, such as to two or three or more different targets or antigens. The term “bispecific” herein refers to a molecule such as a binding protein or antibody that is able to specifically bind to two different targets or antigens. A “multispecific” or “bispecific” antibody herein may include the appropriate full length heavy and light chains for binding to two different antigens, or it may include appropriate antibody fragments for binding to two different antigens.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “nucleic acid molecule” or “nucleic acid” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi:10.1038/nm.4356 or EP 2 101 823 B1). In some embodiments herein, a nucleic acid molecule encodes a recombinant antibody.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” refers to one or more nucleic acid molecules encoding antibody heavy and light chains of antibodies herein (or fragments thereof), including such nucleic acid molecule(s) in a single vector or separate vectors, and such nucleic acid molecule(s) present at one or more locations in a host cell.

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The terms “host cell”. “host cell line”, and “host cell culture” are used interchangeably and refer to cells into which at least one exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell that is “isolated” is one that is separated from a natural environment, for example, present in a laboratory such as in a cell culture system, or for example, otherwise being in vitro or ex vivo, as opposed to being in its natural in vivo environment.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasts.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

By “reduce” is meant the ability to cause an overall decrease. In some embodiments, reduce or inhibit can refer to a relative reduction compared to a reference (e.g., reference level of biological activity or binding affinity, such as lipase binding affinity). In some embodiments, the binding affinity is reduced by at least 2-fold, for example, or by a larger degree, such as at least 5-fold, at least 10-fold, at least 20-fold, at least 30-fold, at least 50-fold, or at least 100-fold. Note that a reduction in binding affinity may be measured by an increase in the dissociation constant, or K_{D}, between the molecules, or an increase in the IC50, for example, as measured by an assay such as surface plasmon resonance (SPR), microscale thermophoresis (MST), or ELISA.

As used herein, the term “about” refers to a numeric value, including, for example, whole numbers, fractions, and percentages, whether or not explicitly indicated. The term “about” generally refers to a range of numerical values (e.g., +/−5-10% of the recited range) that one of ordinary skill in the art would consider equivalent to the recited value (e.g., having the same function or result). When terms such as “at least” and “about” precede a list of numerical values or ranges, the terms modify all of the values or ranges provided in the list. In some instances, the term about may include numerical values that are rounded to the nearest significant figure.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

Exemplary techniques used in connection with recombinant DNA, oligonucleotide synthesis, tissue culture and transformation (e.g., electroporation, lipofection), enzymatic reactions, and purification techniques are described, e.g., in Sambrook et at Molecular Cloning: A Laboratory Manual (2nd ed., Cold Spring Harbor Laboratory Press, Cold Spring Harbor, N.Y. (1989)), among other places.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

An “individual” or “subject” is a human unless otherwise specified. In some cases, where specified, an “individual” or “subject” is a non-human mammal or includes non-human mammals (e.g., “a mammalian subject” or a “non-human mammal subject”). Mammals include, but are not limited to, domesticated animals (e.g., cows, sheep, cats, dogs, and horses), primates (e.g., humans and non-human primates such as monkeys), rabbits, and rodents (e.g., mice and rats).

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

An “effective amount” of an agent, e.g., a pharmaceutical composition comprising an antibody, refers to an amount effective, at dosages and for periods of time necessary, to achieve the desired therapeutic or prophylactic result.

### Exemplary Antibodies

The present disclosure relates in part to antibodies that arc modified to alter the interactions of the antibody with one or more endogenous lipases expressed by host cells used to express the antibodies. For example, many antibodies are produced in high concentration in host cells in cell culture, and then are isolated and purified from the cell culture medium. Host cells, however, also produce endogenous proteins that could, depending on conditions, be retained in low or trace amounts in the isolated and purified antibody formulations, for example, because they may interact with the antibodies or may co-purify with the antibodies, or may persist with the protein intended to be purified through other mechanisms. Such host cell protein impurities, even at very low, trace levels, could trigger immune responses in patients, where the antibody is used therapeutically, and could also shorten the shelf-life of an antibody, reduce its potency, or destabilize an antibody formulation, particularly in cases where the antibody is formulated at a very high concentration. An example of such potential host cell proteins that may end up being retained in antibodies isolated from host cells are endogenous lipase proteins such as, for example, lipase phospholipase B like protein (PLBL2) and lysosomal phospholipase A2 (LPLA2), which are expressed, for instance, in host cells such as Chinese hamster ovary (CHO) cells.

The present disclosure relates in part to antibodies that are modified to alter or reduce interactions with endogenous lipases from host cells, such as any one or more of the lipases listed in the “Exemplary Lipases” section below and the table therein, which includes PLBL2, LPLA2, and others.

In some embodiments, the antibody is modified to alter its glycosylation profile. In some embodiments, the antibody is modified in its constant region, such as the CH1, CH2, and/or CH3 region, to reduce interactions with exogenous lipases. For example, the Examples herein describe that human IgG4 antibodies may interact with endogenous lipases of host cells in the CH1 region, such as in a portion from P149 to S197, and that human IgG1 antibodies may interact with endogenous lipases of host cells in the CH1 region, such as in a portion from V152 to P214 (Kabat numbering). Thus, in some embodiments, one or more amino acid residues within those stretches of the CH1 region may be modified (i.e., by substitution, insertion, or deletion). In some cases, one or more residues in the CH1 region, or in the P149-S197 portion of a human IgG4 heavy chain constant region, or in the V152-P214 portion of a human IgG1 heavy chain constant region (Kabat numbering), may be substituted with another amino acid residue, such as an alanine, glycine, valine, isoleucine, or the like. In some cases, the substitution is for an alanine, leucine, or isoleucine. In some cases, substitution is for an alanine. In other cases, the substitution is for a phenylalanine, tryptophan, or tyrosine (e.g., the substitution of a smaller residue for a Phe, Trp, or Tyr). In yet other cases, the substitution is for an aspartic acid or glutamic acid (e.g., the substitution of a neutral, basic, or hydrophobic residue for an Asp or Glu). In yet more cases, the substitution is for an arginine or lysine (e.g., the substitution of a neutral, acidic, or hydrophobic residue for an Arg or Lys).

In some embodiments, an antibody is modified such that the modification results in at least a 5-fold, 10-fold, 20-fold, 30-fold, 50-fold, or 100-fold reduction in binding affinity of the antibody to the one or more lipases expressed by the cell, e.g., lipase PLBL2, as determined by determined by surface plasmon resonance (SPR), microscale thermophoresis (MST), and/or ELISA. In another embodiment, the modification is determined to lead to a statistically significant difference in level of binding to at least one endogenous lipase by MS or IM experiments, e.g., resulting in at least a 5-fold, 10-fold, 20-fold, 30-fold, 50-fold, or 100-fold reduction in the level of interaction of said antibody to said one or more endogenous lipases, for example, as determined by binding dissociation as measured by mass spectroscopy, such as electrospray ionization (EST) MS (ESI-MS), (e.g., by VC50) or by the amount of antibody-lipase complexes detected by SEC-MS or atmospheric ion mobility. (See Hofstadler and Sannes-Lowery, Nature Reviews 5: 585-595 (2006), for description of ESI-MS.)

Examples of antibody CH1 modifications include substitutions of any of the following amino acids (one-letter abbreviations and Kabat numbering): G170, V171, T173, F174, P175, V177, L178, Q179, 5180, 5181, 0182, L186, F154, P155, V189, V190, T191, V192, P193, S194, S195, S196, L198, K200, P157, V158, and T159. In some cases, substitutions are in at least one of F174, P175, Q179, V192, L198 and K200. The substitutions may be with any amino acid other than the one originally in that position. For example, the substitution may be with an amino acid selected from the group consisting of alanine (A), leucine (L) and isoleucine (1); in a specific embodiment, the substitution is with alanine (A). As another example, the substitution may be with an amino acid selected from the group consisting of phenylalanine (F), tryptophan (W) and tyrosine (Y); in a specific embodiment, the substitution is with tryptophan (W) or tyrosine (Y). As another example, the substitution may be with an amino acid selected from the group consisting of aspartic acid (D) and glutamic acid (E). As another example, the substitution may be with an amino acid selected from the group consisting of Arginine (R) and Lysine (K).

In some cases, the modification comprises a substitution of at least one amino acid selected from the group consisting of G170, V171, T173, F174, P175, V177, L178, Q179, S180, S181, G182, L186, F154, P155, V189, V190, T191, V192, P193, S194, S195, S196, L198, K200, P157, V158, and T159 (Kabat numbering). In some cases, modification is a substitution of an amino acid selected from the group consisting of F174, P175, Q179, V192, L198 and K200 (Kabat numbering). In some cases, the substitution is at a residue selected from the group consisting of G170A, V171A, T173A, F174A, P175A, V177A, L178A, Q179A, S180A, S181A, G182A, L186A, F154A, P155A, V189A, V190A, T191A, V192A, P193A, S194A, S195A, S196A, L198A, K200A, P157A, V158A, and T159A (Kabat numbering). In some cases the substitution is selected from the group consisting of F174A, P175A, Q179A, V192A, L198A and K200A.

In some cases, the glycosylation modification or amino acid modification of the antibody leads to an at least 2-fold, at least 5-fold, at least 10-fold, at least 20-fold, at least 30-fold, at least 50-fold, or at least 100-fold decrease in binding affinity (K_{D}) compared to the unmodified antibody. (See. e.g., Table 4 below.) For instance, modifications at residues F174, P175, Q179, V192, L198 and K200 (Kabat numbering) of an IgG4 antibody, such as alanine substitutions, may lead to at least a 30-fold increase in K_{D} (i.e., weaker affinity) for one or more lipases, such as PLBL2, for example, by SPR, and in some cases, at least a 50-fold increase. Substitutions at those residues as well as at G182, P155, and V189 (Kabat numbering), such as alanine substitutions, may least to at least a 20-fold increase in K_{D}, such as at least a 25-fold increase. And modifications at F174, P175, Q179, V192, L198, K200, G182, P155, and V189, as well as at V171, T173, V177, L178, 5180, S181, F154, V190, T191, P193, S195, S196, and T159, such as alanine substitutions, may lead to at least a 10-fold increase in K_{D}.

In any of the above cases in some embodiments, only one CH1 residue is substituted. In other embodiments, two CH1 residues are substituted. In other embodiments, three or four CH1 residues are substituted.

In some embodiments, the antibody heavy chain constant region does not comprise a modification in an amino acid selected from residues 203-256 (Kabat numbering), does not comprise a modification in an amino acid selected from residues 203-243 (Kabat numbering), or does not comprise a modification of an amino acid selected from residues 197 and 198 and 203-243, and 246-251 (Kabat numbering). In some cases the antibody is a human IgG4 antibody, and does not comprise a modification in an amino acid selected from any one or more of S197, L198, K203, T207, D211, R222, E226, S227, L229, G230, P237, P238, E246, F247, G249, G250, or P251.

In some embodiments, the antibody does not comprise a modification in the heavy chain constant region other than one or more of those described above. In other cases, however, the heavy chain constant region of the antibody also comprises other modifications, for example, to modify ADCC activity or other properties of the antibody, examples of which are described further below. In some cases, the antibody does not comprise modifications in the CH2 or CH3 region, i.e., in the Fc region, and thus, comprises a wild-type Fc region, such as a wild-type, human Fc region. In other cases, the antibody comprises one or more Fc region modifications, such as those described in the section below.

Thus, in addition to the modifications discussed above, it may be desirable to alter the binding affinity of an antibody for its antigen target and/or to modify other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Because of the modular nature of antibodies, and the fact that the modifications described herein are located in the heavy chain constant region, the modifications herein are compatible with any type of antibody variable region, and may be applied to a wide variety of antibodies with different antigen targets, functions, and CDR and variable region sequences.

### Glycosylation Variants

Glycosylation of the Fc portion of an antibody, as well as certain other amino acid sequence modifications may impact the effector function of an antibody. In certain aspects, the disclosure contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example. Fc receptor (FcR) binding assays can be conducted to ensure that the antibody lacks FcγR binding (hence likely lacking ADCC activity), but retains FcRn binding ability. The primary cells for mediating ADCC. NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom. I et al., Proc. Nat'l Acad. Sci. USA 82:1499-1502 (1985); 5,821,337 (see Bruggemann, M. et al., J. Exp. Med 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad Sci. USA 95:652-656 (1998). C1q binding assays may also be carried out to confirm that the antibody is unable to bind C1q and hence lacks CDC activity. See. e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Intl. Immunol. 18(12):1759-1769 (2006); WO 2013/120929 A1),In certain aspects, an antibody provided herein is further altered to increase or decrease the extent to which the antibody is glycosylated. Addition or deletion of glycosylation sites to an antibody may be conveniently accomplished by altering the amino acid sequence such that one or more glycosylation sites is created or removed. Alteration of certain glycosylation sites may, for example, alter interactions with Fc gamma receptors, and may alter the effector function of an antibody. Such alterations, in some embodiments, may be performed in addition to alterations intended to alter interactions with host cell proteins such as lipases.

For example, native IgG antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See. e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (20114); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, U S2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 20114/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### Fc Region Variants

In certain aspects, one or more additional amino acid modifications may be introduced into the Fc region of an antibody provided herein, thereby generating an Fc region variant. For example, in some aspects, an antibody herein has effector function. In other aspects, an antibody herein lacks effector function. In some aspects, the antibody is further modified to alter effector function.

Antibodies with reduced effector function include those with substitution of one or more of Fc region residues 238, 265, 269, 270, 297, 327 and 329 (U.S. Pat. No. 6,737,056). Such Fe mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspects, an antibody variant comprises an Fc region with one or more amino acid substitutions which improve ADCC, e.g., substitutions at positions 298.333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P3290 in an Fc region derived from a human IgG₁ Fc region. In one aspect, the substitutions are L234A, L235A and P3290 (LALAPG) in an Fc region derived from a human IgG₁ Fc region. (See, e.g., WO 2012/130831). In another aspect, the substitutions are L234A, L235A and D265A (LALA-DA) in an Fc region derived from a human IgG₁ Fc region.

In some aspects, the antibodies may have a modification at position N297 to reduce or eliminate ADCC activity, such as N297G or N297Q. In some such cases, the antibody lacks effector function.

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See. e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues I253, H310, H433, N434, and H435 (EU index numbering) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254. H435, and Y436 are crucial for the interaction (Firan, M., et al., Int. Immunol. 13 (2001) 993; Shields. R. L., et al., J. Biol. Chem. 276 (2001) 6591-6604). In Yeung. Y. A., et at (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 8,30,930, 7,855,275, 9,000,130, or WO 2016040856.

### Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide-ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### Recombinant Methods and Compositions

Proteins herein may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised in one or more nucleic acid molecules or expression vectors.

In one aspect, a method of making a recombinant antibody is provided, wherein the method comprises culturing a host cell comprising nucleic acid(s) encoding the antibody or components of the antibody, as provided above, under conditions suitable for expression of the antibody, and optionally recovering the antibody from the host cell (or host cell culture medium). For recombinant production of an antibody, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

### Exemplary Host Cells and Lipases

Suitable host cells for cloning or expression of protein-encoding vectors include prokaryotic or eukaryotic cells. For example, antibodies may be produced in bacteria, in particular when glycosylation and Fc effector function are not needed. For expression of antibody fragments and polypeptides in bacteria, see. e.g., U.S. Pat. Nos. 5,648,237, 5,789,199, and 5,840,523. (See also Charlton, K. A., In: Methods in Molecular Biology, Vol. 248. Lo, B. K. C. (ed.), Humana Press, Totowa, N.J. (2003), pp. 245-254, describing expression of antibody fragments in E. coli.) After expression, the antibody may be isolated from the bacterial cell paste in a soluble fraction and can be further purified.

Mammalian host cells are often used to express proteins such as antibodies, however, particularly those intended for therapeutic use. For example, mammalian cell lines that are adapted to grow in suspension may be useful. Examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W 138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, N.J. (2004), pp. 255-268.

In one aspect, the host cell is a Chinese Hamster Ovary (CHO) cell or lymphoid cell (e.g., Y0, NS0, Sp20 cell). In some cases, the host cell is a CHO cell. In some cases, the CHO cell is modified, for example, to produce antibodies with an altered glycosylation state.

As described herein, host cells comprise endogenous lipases, which may interact with recombinant proteins or antibodies, leading to low levels of the lipases being included as contaminants in the purified antibodies and in antibody formulations. Below are sequences of exemplary lipases tested herein for Ab-lipase binding, showing predicted glycosylation sites in bold, with exemplary relevant asparagine residues underlined:

| Thioesterase (G3HNG5) glycan positions. 298, 422 | |
|---|---|
| (SEQ ID NO: 1) | |
| MAQRLAPNTPEGFKAVTASLGRPRSTKAQPDSEAMQASTAQDQMAPIMILEPADGCLCDQP | 80 |
| VLISVHGLAPEQPVTLRAA | |
| LRDEKGALFRAHARYRADDHGGLDLARAPALGGSFAGIEPMGLLWALEPERPFWRLIKRDV | 160 |
| QTPFVVELEVLDGHEPDGG | |
| RLLARAVHERHFMAPGVRRVPVREGRVRATLFLPPGNGPFPGIVDLFGVGGGLLEYRASLL | 240 |
| AGKGFAVMALAYYNYDDLP | |
| KGMDIFHLEYFEEAVNYLLSHPQVKGPGIGLLGISKGGELGLAMASFLKGIKAAVIINGSV | 320 |
| AAVGNTIHYKDETIPPVSL | |
| LRNRVKMTKDGLKDVVEGLQSPLVEEKSFIPVERPDTAFLLLVGQDDHNWKSEFYANEISK | 400 |
| RLEAHGKEKPQIICYPEAG | |
| HYIEPPYFPLCKAGMHLLVGANITFGGEPKPHAVAQVDAWQQLQTFFHKHLGGEERTIPSK | |
| L | |
| LPLA2 (Q8NCC3, human sequence shown) glycan | |
| positions, 99, 273, 289, 398 | |
| (SEQ ID NO: 2) | |
| MGLHLRPYRVGLLPDGLLFLLLLLMLLADPALPAGRHPPVVLVPGDLGNQLEAKLDKPTVV | 80 |
| HYLCSKKTESYFTIWLNLE | |
| LLLPVIIDCWIDNIRLVYNKTSRATQFPDGVDVRVPGFGKTFSLEFLDPSKSSVGSYFHTM | 160 |
| VESLVGWGYTRGEDVRGAP | |
| YDWRRAPNENGPYFLALREMIEEMYQLYGGPVVLVAHSMGNMYTLYFLQRQPQAWKDKYIR | 240 |
| AFVSLGAPWGGVAKTLRVL | |
| ASGDNNRIPVIGPLKIREQQRSAVSTSWLLPYNYTWSPEKVFVQTPTINYTLRDYRKFFQD | 320 |
| IGFEDGWLMRQDTEGLVEA | |
| TMPPGVQLHCLYGTGVPTPDSFYYESFPDRDPKICFGDGDGTVNLKSALQCQAWQSRQEHQ | 400 |
| VLLQELPGSEHIEMLANAT | |
| TLAYLKRVLLGP | |
| PLBL2 (G316T1, listed without signal sequence) | |
| glycan positions, 47, 65, 69, 190, 395, 474 | |
| (SEQ ID NO: 3) | |
| LPTQGPGRRRQNLDPPVSRVRSVLLDAASGQLRLVDGIHPYAVAWANLTNAIRETGWAYLD | 80 |
| LGTNGSYNDSLQAYAAGVV | |
| EASVSEELIYMHWMNTMVNYCGPFEYEVGYCEKLKSFLEINLEWMQREMELSQDSPYWHQV | 160 |
| RLTLLQLKGLEDSYEGRLT | |
| FPTGRFTIKPLGFLLLQIAGDLEDLEQALNKTSTKLSLGSGSCSAIIKLLPGARDLLVAHN | 240 |
| TWNSYQNMLRIIKKYQLQF | |
| RQGPQEAYPLIAGNNLVFSSYPGTTFSGDDFYILGSGLVTLETTIGNKNPALWKYVQPQGC | 320 |
| VLEWIRNIVANRLALDGAT | |
| WADIFKQFNSGTYNNQWMIVDYKAFIPNGPSPGSRVLTILEQIPGMVVVADKTEDLYKTTY | 400 |
| WASYNIPFFEIVFNASGIQ | |
| DLVAQYGDWFSYTKNPRAQIFQRDQSLVEDMNSMVRLIRYNNFLHDPLSLCEACIPKPNAE | 480 |
| NAISARSDLNPANGSYPFQ | |
| ALYQRPHGGIDVKVTSFSLAKRMSMLAASGPTWDQLPPFQWSLSPFRSMLHMGQPDLWTFS | |
| PISVPWD | |
| PPT (G3HN89) glycan positions, 197, 212, 232 | |
| (SEQ ID NO: 4) | |
| MASPGSRWLLAVSLLPWCCAAWSLGHLNPPSLTPLVIWHGMGDSCCNPISMGAIKKMVEKE | 80 |
| IPGIYVLSLEIGKNMMEDV | |
| ENSFFLNVNSQVMMVCQILEKDPKLQQGYNAIGFSQGGQFLRAVAQRCPSPRMINLISVGG | 160 |
| QHQGVFGLPRCPGESSHVC | |
| DFIRKMINAGAYSKVVQLRLVQAQYWHDPTKEDVYRNHSIFLADINQERCVNETYKKNLMA | 240 |
| LNKFVMVKFLNDSIVDPVD | |
| SEWFGFYRSGQAKETIPLQESTLYTEDRLGLKQMDKAGKLVFLAKEGDHLQLSKEWFNAYI | |
| IPFL | |
| PLD3 (G3HNQ5) glycan positions 97, 102, 132, | |
| 234, 282, 385, 430 | |
| (SEQ ID NO: 5) | |
| MKPKLMYQELKVPVEEPAGELPVNEIEAWKAAEKKARWVLLVLILAVVGFGALMTQLFLWE | 80 |
| YGDLHLFGPNQRPAPCYDP | |
| CEAVLVESIPEGLEFPNATTSNPSTSQAWLGLLAGAHSSLDIASFYWTLTNNDTHTQEPSA | 160 |
| QQGEEILQQLQALAPRGVK | |
| VRIAVSKPNGPLADLQSLLQSGAQVRMVDMQKLTHGVLHTKFWVVDQTHFYLGSANMDWRS | 240 |
| LTQVKELGVVMYNCSCLAR | |
| DLTKIFEAYWFLGQAGSSIPSTWPRPFDTRYNQETPMEICLNGTPALAYLASAPPPLCPSG | 320 |
| RTPDLKALLSVVDSARSFI | |
| YIAVMNYLPTMEFSHPRRFWPAIDDGLRRAAYERGVKVRLLVSCWGHSEPSMRSFLLSLAA | 400 |
| LRDNHTHSDIQVKLFVVPA | |
| DEAQARIPYARVNHNKYMVTERAVYIGTSNWSGSYFTETAGTSLLVTQNGHDGLRSQLEDV | 480 |
| FLRDWNSLYSHNLDTAADS | |
| VGNACRLL | |
| SP (A0A061IEQ5) glycan positions 84, 173, 333, | |
| 393, 518, 611 | |
| (SEQ ID NO: 6) | |
| MPRHGVSSGQGHLRADLEIRLESSLPAPKLGLLWMGLVLALVLTLFDSTVLWTPARAYPLP | 80 |
| AQGHPVKFSAIVPPLQNAF | |
| VWRNLSCPACKVLFTALNYGLKKEPNVARVGSVAIKMCKMLNIAPLNVCQSAVHLFEDDVV | 160 |
| EVWTRSVLSPSEACGLLLG | |
| PSCGHWDIFSSWNISLPSVPKPPPKPPSPPAPGAPVSRVLFLTDLHWDHDYLEGTDPNCAD | 240 |
| PLCCRRSSGWPPNSQAGAG | |
| YWGEYSKCDLPLRTLESLLKGLGPAGPFEMVYWTGDIPAHDVWQQSRQDQLRALTTVTDLV | 320 |
| RKFLGPVPVYPAVGNHEST | |
| PVNGFPPPFIKGNQSSQWLYEAMAKAWEPWLPADALHTLRIGGFYALTPRPGLRLISLNMN | 400 |
| FCSRENFWLLINSTDPAGQ | |
| LQWLVEELQAAENRGDKVHIIGHIPPGHCLKSWSWNYYKIVARYENTLAGQFFGHTHVDEF | 480 |
| EIFYDEETLSRPLAVAFLG | |
| PSATTYINLNPGYRVYQIDGNYPGSSHVVLDHETYILNLTQANAPEATPHWKRLYRARETY | 560 |
| GLPDALPASWHNLVYRMRD | |
| NEQLFQTFWFLYHKGHPPSEPCGTPCRLATLCAQLSARADSPALCRHLMPNGSLPDAHSLW | |
| SRTLLC | |

Additional exemplary lipases include Clusterin. Lipoprotein lipase, Patatin-like phospholipase domain-containing protein 5, phospholipase DDHD1, lysophospholipase. Monoacylglycerol lipase ABHD12, Lysophospholipase-like protein 1, Lysosomal acid lipase/cholesteryl ester hydrolase, Lipase maturation factor 2, Patatin-like phospholipase domain-containing protein 5, Monoacylglycerol lipase ABHD12, ans Lysosomal acid lipase/cholesteryl ester hydrolase.

Also included are lipases summarized in the table below:

| Uniprot | Entry | Protein | Gene | | |
|---|---|---|---|---|---|
| Entry | name | names | names | Organism | Length |
| A0A3L7H5I5 | A0A3L7H5I5_CRIGR | Hormone- | CgPICR_018531 | Cricetulus griseus | 843 |
| | | sensitive | | (Chinese hamster) | |
| | | lipase | | (Cricetulus | |
| | | | | barabensis griseus) | |
| A0A061HW32 | A0A061HW32_CRIGR | Hormone- | H671_20969 | Cricetulus griseus | 592 |
| | | sensitive | | (Chinese hamster) | |
| | | lipase | | (Cricetulus | |
| | | | | barabensis griseus) | |
| A0A3L7HAZ7 | A0A3L7HAZ7_CRIGR | Lysophospho | CgPICR_006350 | Cricetulus griseus | 1,439 |
| | | lipase | | (Chinese hamster) | |
| | | | | (Cricetulus | |
| | | | | barabensis griseus) | |
| A0A3L7IKX6 | A0A3L7IKX6_CRIGR | Lipoprotein | CgPICR_019757 | Cricetulus griseus | 474 |
| | | lipase | | (Chinese hamster) | |
| | | | | (Cricetulus | |
| | | | | barabensis griseus) | |
| G3GRM1 | G3GRM1_CRIGR | Hepatic | I79_000173 | Cricetulus griseus | 470 |
| | | triacylglycerol | | (Chinese hamster) | |
| | | lipase | | (Cricetulus | |
| | | | | barabensis griseus) | |
| A0A061I1V4 | A0A061I1V4_CRIGR | Hepatic | H671_4g13339 | Cricetulus griseus | 412 |
| | | triacylglycerol | | (Chinese hamster) | |
| | | lipase | | (Cricetulus | |
| | | | | barabensis griseus) | |
| A0A3L7HTB5 | A0A3L7HTB5_CRIGR | Hepatic | CgPICR_005213 | Cricetulus griseus | 502 |
| | | triacylglycerol | | (Chinese hamster) | |
| | | lipase | | (Cricetulus | |
| | | | | barabensis griseus) | |
| A0A061IKA1 | A0A061IKA1_CRIGR | Lipoprotein | H671_1g2493 | Cricetulus griseus | 487 |
| | | lipase | | (Chinese hamster) | |
| | | | | (Cricetulus | |
| | | | | barabensis griseus) | |
| G3H6V7 | G3H6V7_CRIGR | Lipoprotein | I79_006077 | Cricetulus griseus | 450 |
| | | lipase | | (Chinese hamster) | |
| | | | | (Cricetulus | |
| | | | | barabensis griseus) | |
| A0A3L7HY34 | A0A3L7HY34_CRIGR | Triacylglycerol | CgPICR_007694 | Cricetulus griseus | 469 |
| | | lipase | | (Chinese hamster) | |
| | | | | (Cricetulus | |
| | | | | barabensis griseus) | |
| A0A3L7HXX8 | A0A3L7HXX8_CRIGR | Triacylglycerol | CgPICR_007698 | Cricetulus griseus | 466 |
| | | lipase | | (Chinese hamster) | |
| | | | | (Cricetulus | |
| | | | | barabensis griseus) | |
| G3H6Z2 | G3H6Z2_CRIGR | 2-arachidonoyl | I79_006116 | Cricetulus griseus | 300 |
| | | glycerol | | (Chinese hamster) | |
| | | hydrolase | | (Cricetulus | |
| | | AB . . . | | barabensis griseus) | |
| A0A061IL50 | A0A061IL50_CRIGR | ABHD6 | CgPICR_004910, | Cricetulus griseus | 337 |
| | | | H671_1g3077 | (Chinese hamster) | |
| | | | | (Cricetulus | |
| | | | | barabensis griseus) | |
| G3HQY6 | G3HQY6_CRIGR | Lipase | I79_013245 | Cricetulus griseus | 397 |
| | | | | (Chinese hamster) | |
| | | | | (Cricetulus | |
| | | | | barabensis griseus) | |
| G31FK5 | G3IFK5_CRIGR | DAGLA | CgPICR_006759, | Cricetulus griseus | 1,043 |
| | | | I79_022528 | (Chinese hamster) | |
| | | | | (Cricetulus | |
| | | | | barabensis griseus) | |
| G3IL04 | G3IL04_CRIGR | Triacylglycerol | I79_024562 | Cricetulus griseus | 758 |
| | | lipase | | (Chinese hamster) | |
| | | | | (Cricetulus | |
| | | | | barabensis griseus) | |
| G3IN33 | G3IN33_CRIGR | Triacylglycerol | I79_025345 | Cricetulus griseus | 463 |
| | | lipase | | (Chinese hamster) | |
| | | | | (Cricetulus | |
| | | | | barabensis griseus) | |
| A0A3L7HXU7 | A0A3L7HXU7_CRIGR | Triacylglycerol | CgPICR_007696 | Cricetulus griseus | 476 |
| | | lipase | | (Chinese hamster) | |
| | | | | (Cricetulus | |
| | | | | barabensis griseus) | |
| AUA061I462 | A0A061I462_CRIGR | Apolipoprotein | H671_4g12453 | Cricetulus griseus | 414 |
| | | A-IV-like | | (Chinese hamster) | |
| | | protein | | (Cricetulus | |
| | | | | barabensis griseus) | |
| A0A3L7HYQ8 | A0A3L7HYQ8_CRIGR | Lipase | CgPICR_006565 | Cricetulus griseus | 396 |
| | | | | (Chinese hamster) | |
| | | | | (Cricetulus | |
| | | | | barabensis griseus) | |
| A0A3L7I0B5 | A0A3L7I0B5_CRIGR | Lipase | CgPICR_006567 | Cricetulus griseus | 399 |
| | | | | (Chinese hamster) | |
| | | | | (Cricetulus | |
| | | | | barabensis griseus) | |
| A0A3L7HYQ0 | A0A3L7HYQ0_CRIGR | Gastric | CgPICR_006570 | Cricetulus griseus | 441 |
| | | triacylglycerol | | (Chinese hamster) | |
| | | lipase | | (Cricetulus | |
| | | | | barabensis griseus) | |
| G3I731 | G3I731_CRIGR | NR1H2 | CgPICR_019340, | Cricetulus griseus | 445 |
| | | | I79_019312 | (Chinese hamster) | |
| | | | | (Cricetulus | |
| | | | | barabensis griseus) | |
| A0A3L7HAG1 | A0A3L7HAG1_CRIGR | Apolipoprotein H | CgPICR_012123 | Cricetulus griseus | 345 |
| | | | | (Chinese hamster) | |
| | | | | (Cricetulus | |
| | | | | barabensis griseus) | |
| A0A3L7GMF6 | A0A3L7GMF6_CRIGR | NLGN2 | CgPICR_003667 | Cricetulus griseus | 836 |
| | | | | (Chinese hamster) | |
| | | | | (Cricetulus | |
| | | | | barabensis griseus) | |
| A0A3L7IAJ0 | A0A3L7IAJ0_CRIGR | Lipase | CgPICR_000068 | Cricetulus griseus | 704 |
| | | maturation | | (Chinese hamster) | |
| | | factor | | (Cricetulus | |
| | | | | barabensis griseus) | |
| G3IKG6 | G3IKG6_CRIGR | Triacylglycerol | I79_024369 | Cricetulus griseus | 386 |
| | | lipase | | (Chinese hamster) | |
| | | | | (Cricetulus | |
| | | | | barabensis griseus) | |
| G3HQJ0 | G3HQJ0_CRIGR | Phospholipase | I79_013092 | Cricetulus griseus | 543 |
| | | A1 | | (Chinese hamster) | |
| | | | | (Cricetulus | |
| | | | | barabensis griseus) | |
| A0A3L7IBZ9 | A0A3L7IBZ9_CRIGR | Phospholipase A1 | CgPICR_017566 | Cricetulus griseus | 494 |
| | | | | (Chinese hamster) | |
| | | | | (Cricetulus | |
| | | | | barabensis griseus) | |
| G3I2W3 | G3I2W3_CRIGR | Lipase | I79_017758 | Cricetulus griseus | 699 |
| | | maturation | | (Chinese hamster) | |
| | | factor | | (Cricetulus | |
| | | | | barabensis griseus) | |
| G3HBV6 | G3HBV6_CRIGR | Lipase | I79_007907 | Cricetulus griseus | 355 |
| | | maturation | | (Chinese hamster) | |
| | | factor | | (Cricetulus | |
| | | | | barabensis griseus) | |
| A0A061I960 | A0A061I960_CRIGR | Lipase | CgPICR_006562, | Cricetulus griseus | 422 |
| | | | H671_3g9853 | (Chinese hamster) | |
| | | | | (Cricetulus | |
| | | | | barabensis griseus) | |
| A0A3L7HYR2 | A0A3L7HYR2_CRIGR | Lipase | CgPICR_006563 | Cricetulus griseus | 400 |
| | | | | (Chinese hamster) | |
| | | | | (Cricetulus | |
| | | | | barabensis griseus) | |
| A0A3L7HZD7 | A0A3L7HZD7_CRIGR | Lipase | CgPICR_006556 | Cricetulus griseus | 402 |
| | | | | (Chinese hamster) | |
| | | | | (Cricetulus | |
| | | | | barabensis griseus) | |
| G3GYM8 | G3GYM8_CRIGR | Fatty acid | I79_002933 | Cricetulus griseus | 579 |
| | | amide | | (Chinese hamster) | |
| | | hydrolase | | (Cricetulus | |
| | | | | barabensis griseus) | |
| G3I5R0 | G3I5R0_CRIGR | ANGPTL4 | CgPICR_001128, | Cricetulus griseus | 411 |
| | | C-terminal | I79_018815 | (Chinese hamster) | |
| | | chain | | (Cricetulus | |
| | | | | barabensis griseus) | |
| A0A3L7HYM5 | A0A3L7HYM5_CRIGR | Abhydro_lipase | CgPICR_006574 | Cricetulus griseus | 359 |
| | | domain- | | (Chinese hamster) | |
| | | containing | | (Cricetulus | |
| | | protein | | barabensis griseus) | |
| A0A061HX37 | A0A061HX37_CRIGR | Mediator of | H671_xg20514 | Cricetulus griseus | 2,793 |
| | | RNA | | (Chinese hamster) | |
| | | polymerase II | | (Cricetulus | |
| | | trans . . . | | barabensis griseus) | |
| G3IAQ4 | G3IAQ4_CRIGR | Abhydrolase | I79_020680 | Cricetulus griseus | 425 |
| | | domain- | | (Chinese hamster) | |
| | | containing | | (Cricetulus | |
| | | protein | | barabensis griseus) | |
| G3HKV9 | G3HKV9_CRIGR | Group XV | CgPICR_005649, | Cricetulus griseus | 412 |
| | | phospholipase | I79_011341 | (Chinese hamster) | |
| | | A2 | | (Cricetulus | |
| | | | | barabensis griseus) | |
| G3HZU4 | G3HZU4_CRIGR | Colipase | CgPICR_020526, | Cricetulus griseus | 113 |
| | | | I79_016608 | (Chinese hamster) | |
| | | | | (Cricetulus | |
| | | | | barabensis griseus) | |
| G3HTY7 | G3HTY7_CRIGR | Apolipoprotein | H671_5g13744, | Cricetulus griseus | 101 |
| | | A-II | I79_014389 | (Chinese hamster) | |
| | | | | (Cricetulus | |
| | | | | barabensis griseus) | |
| A0A3L7HYV3 | A0A3L7HYV3_CRIGR | Abhydro_lipase | CgPICR_006569 | Cricetulus griseus | 386 |
| | | domain- | | (Chinese hamster) | |
| | | containing | | (Cricetulus | |
| | | protein | | barabensis griseus) | |
| G3HBV7 | G3HBV7_CRIGR | Lipase | I79_007908 | Cricetulus griseus | 168 |
| | | maturation | | (Chinese hamster) | |
| | | factor 1 | | (Cricetulus | |
| | | | | barabensis griseus) | |
| G3IIM8 | G3IIM8_CRIGR | Carboxylic | I79_023701 | Cricetulus griseus | 646 |
| | | ester | | (Chinese hamster) | |
| | | hydrolase | | (Cricetulus | |
| | | | | barabensis griseus) | |
| G3I7K0 | G3I7K0_CRIGR | Neuroligin-3 | I79_019492 | Cricetulus griseus | 828 |
| | | | | (Chinese hamster) | |
| | | | | (Cricetulus | |
| | | | | barabensis griseus) | |
| A0A3L7HDS6 | A0A3L7HDS6_CRIGR | Carboxylic | CgPICR_012466 | Cricetulus griseus | 654 |
| | | ester | | (Chinese hamster) | |
| | | hydrolase | | (Cricetulus | |
| | | | | barabensis griseus) | |
| A0A061IKL2 | A0A061IKL2_CRIGR | Carboxylic | H671_1g2413 | Cricetulus griseus | 603 |
| | | ester | | (Chinese hamster) | |
| | | hydrolase | | (Cricetulus | |
| | | | | barabensis griseus) | |
| A0A3L7H4J2 | A0A3L7H4J2_CRIGR | NLGN3 | CgPICR_018248 | Cricetulus griseus | 841 |
| | | | | (Chinese hamster) | |
| | | | | (Cricetulus | |
| | | | | barabensis griseus) | |
| A0A061I7T5 | A0A061I7T5_CRIGR | Sn1-specific | H671_4g11902 | Cricetulus griseus | 537 |
| | | diacylglycerol | | (Chinese hamster) | |
| | | lipase . . . | | (Cricetulus | |
| | | | | barabensis griseus) | |
| A0A061I7P8 | A0A061I7P8_CRIGR | Lipase | H671_3g10704 | Cricetulus griseus | 402 |
| | | member K | | (Chinese hamster) | |
| | | | | (Cricetulus | |
| | | | | barabensis griseus) | |
| Q8R4V8 | Q8R4V8_CRIGR | Phospholipase | | Cricetulus griseus | 200 |
| | | A1 | | (Chinese hamster) | |
| | | | | (Cricetulus | |
| | | | | barabensis griseus) | |
| G313H3 | G3I3H3_CRIGR | PNPLA2 | CgPICR_009656, | Cricetulus griseus | 486 |
| | | | I79_017981 | (Chinese hamster) | |
| | | | | (Cricetulus | |
| | | | | barabensis griseus) | |
| A0A061IDI1 | A0A061IDI1_CRIGR | Lipase | H671_3g10468 | Cricetulus griseus | 187 |
| | | member K- | | (Chinese hamster) | |
| | | like protein | | (Cricetulus | |
| | | | | barabensis griseus) | |
| A0A3L7HYM1 | A0A3L7HYM1_CRIGR | Abhydro_lipase | CgPICR_006571 | Cricetulus griseus | 229 |
| | | domain- | | (Chinese hamster) | |
| | | containing | | (Cricetulus | |
| | | protein | | barabensis griseus) | |
| A0A3L7HZE7 | A0A3L7HZE7_CRIGR | Abhydro_lipase | CgPICR_006566 | Cricetulus griseus | 272 |
| | | domain- | | (Chinese hamster) | |
| | | containing | | (Cricetulus | |
| | | protein | | barabensis griseus) | |
| A0A061I822 | A0A061I822_CRIGR | Lipase | H671_3g9852 | Cricetulus griseus | 327 |
| | | member N | | (Chinese hamster) | |
| | | | | (Cricetulus | |
| | | | | barabensis griseus) | |
| A0A061HWK1 | A0A061HWK1_CRIGR | Hormone- | H671_21698 | Cricetulus griseus | 196 |
| | | sensitive | | (Chinese hamster) | |
| | | lipase-like | | (Cricetulus | |
| | | protein | | barabensis griseus) | |
| G3HKM8 | G3HKM8_CRIGR | Lipase | I79_01I257 | Cricetulus griseus | 347 |
| | | member H | | (Chinese hamster) | |
| | | | | (Cricetulus | |
| | | | | barabensis griseus) | |
| G3H1I9 | G3H1I9_CRIGR | Phospholipase | I79_004016 | Cricetulus griseus | 697 |
| | | DDHD2 | | (Chinese hamster) | |
| | | | | (Cricetulus | |
| | | | | barabensis griseus) | |
| G3GY86 | G3GY86_CRIGR | Neuroligin-2 | I79_002759 | Cricetulus griseus | 635 |
| | | | | (Chinese hamster) | |
| | | | | (Cricetulus | |
| | | | | barabensis griseus) | |
| G3IGT0 | G3IGT0_CRIGR | Carboxylic | I79_023006 | Cricetulus griseus | 575 |
| | | ester | | (Chinese hamster) | |
| | | hydrolase | | (Cricetulus | |
| | | | | barabensis griseus) | |
| G3H6H1 | G3H6H1_CRIGR | Arylacetamide | I79_005931 | Cricetulus griseus | 408 |
| | | deacetylase- | | (Chinese hamster) | |
| | | like 1 | | (Cricetulus | |
| | | | | barabensis griseus) | |
| A0A3L7IPE5 | A0A3L7IPE5_CRIGR | Thyroglobulin | CgPICR_011560 | Cricetulus griseus | 3,665 |
| | | | | (Chinese hamster) | |
| | | | | (Cricetulus | |
| | | | | barabensis griseus) | |
| A0A3L7H125 | A0A3L7H125_CRIGR | NLGN4X | CgPICR_023230 | Cricetulus griseus | 826 |
| | | | | (Chinese hamster) | |
| | | | | (Cricetulus | |
| | | | | barabensis griseus) | |
| A0A3L7HZX3 | A0A3L7HZX3_CRIGR | CES2 | CgPICR_005602 | Cricetulus griseus | 1,012 |
| | | | | (Chinese hamster) | |
| | | | | (Cricetulus | |
| | | | | barabensis griseus) | |
| A0A061HVH1 | A0A061HVH1_CRIGR | Hormone- | H671_21249 | Cricetulus griseus | 335 |
| | | sensitive | | (Chinese hamster) | |
| | | lipase-like | | (Cricetulus | |
| | | protein | | barabensis griseus) | |
| G3HEB4 | G3HEB4_CRIGR | Neuroligin-1 | I79_008895 | Cricetulus griseus | 385 |
| | | | | (Chinese hamster) | |
| | | | | (Cricetulus | |
| | | | | barabensis griseus) | |
| A0A061I8R9 | A0A061I8R9_CRIGR | Carboxylic | H671_3g9584 | Cricetulus griseus | 275 |
| | | ester | | (Chinese hamster) | |
| | | hydrolase | | (Cricetulus | |
| | | | | barabensis griseus) | |
| A0A3L7IH61 | A0A3L7IH61_CRIGR | NLGN1 | CgPICR_002421 | Cricetulus griseus | 611 |
| | | | | (Chinese hamster) | |
| | | | | (Cricetulus | |
| | | | | barabensis griseus) | |
| G3I1J5 | G3I1J5_CRIGR | Phospholipase | I79_017256 | Cricetulus griseus | 442 |
| | | A1 member A | | (Chinese hamster) | |
| | | | | (Cricetulus | |
| | | | | barabensis griseus) | |
| A0A3L7HXV9 | A0A3L7HXV9_CRIGR | PNLIP | CgPICR_007697 | Cricetulus griseus | 428 |
| | | | | (Chinese hamster) | |
| | | | | (Cricetulus | |
| | | | | barabensis griseus) | |
| A0A3L7HR81 | A0A3L7HR81_CRIGR | PRA1A | CgPICR_002041 | Cricetulus griseus | 442 |
| | | | | (Chinese hamster) | |
| | | | | (Cricetulus | |
| | | | | barabensis griseus) | |
| A0A3L7HQI6 | A0A3L7HQI6_CRIGR | LIPI | CgPICR_001909 | Cricetulus griseus | 520 |
| | | | | (Chinese hamster) | |
| | | | | (Cricetulus | |
| | | | | barabensis griseus) | |
| A0A3L7IJN1 | A0A3L7IJN1_CRIGR | AADAC | CgPICR_009978 | Cricetulus griseus | 398 |
| | | | | (Chinese hamster) | |
| | | | | (Cricetulus | |
| | | | | barabensis griseus) | |
| G3I5L2 | G315L2_CRIGR | Carboxylic | I79_018765 | Cricetulus griseus | 507 |
| | | ester | | (Chinese hamster) | |
| | | hydrolase | | (Cricetulus | |
| | | | | barabensis griseus) | |
| G3HQX5 | G3HQX5_CRIGR | Gastric | I79_013234 | Cricetulus griseus | 302 |
| | | triacylglycerol | | (Chinese hamster) | |
| | | lipase | | (Cricetulus | |
| | | | | barabensis griseus) | |
| G3HQX8 | G3HQX8_CRIGR | Gastric | I79_013237 | Cricetulus griseus | 299 |
| | | triacylglycerol | | (Chinese hamster) | |
| | | lipase | | (Cricetulus | |
| | | | | barabensis griseus) | |
| A0A061I8V8 | A0A061I8V8_CRIGR | Carboxylic | H671_4g12801 | Cricetulus griseus | 461 |
| | | ester | | (Chinese hamster) | |
| | | hydrolase | | (Cricetulus | |
| | | | | barabensis griseus) | |
| A0A061I9X1 | A0A061I9X1_CRIGR | Carboxylic | H671_3g11044 | Cricetulus griseus | 551 |
| | | ester | | (Chinese hamster) | |
| | | hydrolase | | (Cricetulus | |
| | | | | barabensis griseus) | |
| A0A061IAT4 | A0A061IAT4_CRIGR | Carboxylic | H671_3g11044 | Cricetulus griseus | 560 |
| | | ester | | (Chinese hamster) | |
| | | hydrolase | | (Cricetulus | |
| | | | | barabensis griseus) | |
| A0A061I5M8 | A0A061I5M8_CRIGR | Protein ADP- | H671_4g13178 | Cricetulus griseus | 797 |
| | | ribosylarginine | | (Chinese hamster) | |
| | | hydrola . . . | | (Cricetulus | |
| | | | | barabensis griseus) | |
| G3IIG1 | G3IIG1_CR1GR | Carboxylic | I79_023630 | Cricetulus griseus | 561 |
| | | ester | | (Chinese hamster) | |
| | | hydrolase | | (Cricetulus | |
| | | | | barabensis griseus) | |
| G3I766 | G3I766_CRIGR | Carboxylic | I79_019351 | Cricetulus griseus | 561 |
| | | ester | | (Chinese hamster) | |
| | | hydrolase | | (Cricetulus | |
| | | | | barabensis griseus) | |
| A0A061I8D9 | A0A061I8D9_CRIGR | Carboxylic | CgPICR_005503, | Cricetulus griseus | 575 |
| | | ester | H671_3g10206 | (Chinese hamster) | |
| | | hydrolase | | (Cricetulus | |
| | | | | barabensis griseus) | |
| A0A061ID38 | A0A0611D38_CRIGR | Acyloxyacyl | H671_3g9294 | Cricetulus griseus | 575 |
| | | hydrolase | | (Chinese hamster) | |
| | | isoform 1 | | (Cricetulus | |
| | | | | barabensis griseus) | |
| G3IIG0 | G3IIG0_CRIGR | Carboxylic | I79_023629 | Cricetulus griseus | 529 |
| | | ester | | (Chinese hamster) | |
| | | hydrolase | | (Cricetulus | |
| | | | | barabensis griseus) | |
| A0A061I9Z6 | A0A061I9Z6_CRIGR | Carboxylic | H671_3g9073 | Cricetulus griseus | 560 |
| | | ester | | (Chinese hamster) | |
| | | hydrolase | | (Cricetulus | |
| | | | | barabensis griseus) | |
| A0A061IEJ2 | A0A061IEJ2_CRIGR | Carboxylic | CgPICR_005591, | Cricetulus griseus | 558 |
| | | ester | H671_3g9072 | (Chinese hamster) | |
| | | hydrolase | | (Cricetulus | |
| | | | | barabensis griseus) | |
| A0A061I734 | A0A061I734_CRIGR | Carboxylic | CgPICR_005604, | Cricetulus griseus | 564 |
| | | ester | H671_3g11292 | (Chinese hamster) | |
| | | hydrolase | | (Cricetulus | |
| | | | | barabensis griseus) | |
| G3I7X7 | G3I7X7_CRIGR | Carboxylic | I79_019629 | Cricetulus griseus | 564 |
| | | ester | | (Chinese hamster) | |
| | | hydrolase | | (Cricetulus | |
| | | | | barabensis griseus) | |
| A0A061IBF9 | A0A061IBF9_CRIGR | Carboxylic | H671_3g10418 | Cricetulus griseus | 559 |
| | | ester | | (Chinese hamster) | |
| | | hydrolase | | (Cricetulus | |
| | | | | barabensis griseus) | |
| A0A061I6L1 | A0A061I6L1_CRIGR | Carboxylic | H671_3g10650 | Cricetulus griseus | 558 |
| | | ester | | (Chinese hamster) | |
| | | hydrolase | | (Cricetulus | |
| | | | | barabensis griseus) | |
| G3HQX3 | G3HQX3_CRIGR | Gastric | I79_013232 | Cricetulus griseus | 248 |
| | | triacylglycerol | | (Chinese hamster) | |
| | | lipase | | (Cricetulus | |
| | | | | barabensis griseus) | |
| A0A3L7I1B2 | A0A3L7I1B2_CRIGR | Carboxylic | CgPICR_005498 | Cricetulus griseus | 583 |
| | | ester | | (Chinese hamster) | |
| | | hydrolase | | (Cricetulus | |
| | | | | barabensis griseus) | |
| A0A061I5X0 | A0A06115X0_CRIGR | Cocaine | H671_3g11294 | Cricetulus griseus | 1,189 |
| | | esterase-like | | (Chinese hamster) | |
| | | isoform 1 | | (Cricetulus | |
| | | | | barabensis griseus) | |
| A0A3L7HZW4 | A0A3L7HZW4_CRIGR | Carboxylic | CgPICR_005592 | Cricetulus griseus | 560 |
| | | ester | | (Chinese hamster) | |
| | | hydrolase | | (Cricetulus | |
| | | | | barabensis griseus) | |
| G3I5K6 | G3I5K6_CRIGR | Carboxylic | I79_018759 | Cricetulus griseus | 516 |
| | | ester | | (Chinese hamster) | |
| | | hydrolase | | (Cricetulus | |
| | | | | barabensis griseus) | |
| A0A3L7I011 | A0A3L7I011_CRIGR | Carboxylic | CgPICR_005593 | Cricetulus griseus | 549 |
| | | ester | | (Chinese hamster) | |
| | | hydrolase | | (Cricetulus | |
| | | | | barabensis griseus) | |
| A0A061I9H4 | A0A061I9H4_CRIGR | Carboxylic | H671_3g11292 | Cricetulus griseus | 498 |
| | | ester | | (Chinese hamster) | |
| | | hydrolase | | (Cricetulus | |
| | | | | barabensis griseus) | |
| A0A3L7I042 | A0A3L7I042_CRIGR | Carboxylic | CgPICR_005590 | Cricetulus griseus | 560 |
| | | ester | | (Chinese hamster) | |
| | | hydrolase | | (Cricetulus | |
| | | | | barabensis griseus) | |
| A0A061IAM3 | A0A061IAM3_CRIGR | Carboxylic | H671_3g10323 | Cricetulus griseus | 529 |
| | | ester | | (Chinese hamster) | |
| | | hydrolase | | (Cricetulus | |
| | | | | barabensis griseus) | |
| A0A3L7HZM5 | A0A3L7HZM5_CRIGR | Carboxylic | CgPICR_005502 | Cricetulus griseus | 578 |
| | | ester | | (Chinese hamster) | |
| | | hydrolase | | (Cricetulus | |
| | | | | barabensis griseus) | |
| A0A3L7I2N6 | A0A3L7I2N6_CRIGR | Carboxylic | CgPICR_005599 | Cricetulus griseus | 449 |
| | | ester | | (Chinese hamster) | |
| | | hydrolase | | (Cricetulus | |
| | | | | barabensis griseus) | |
| A0A3L7I0Q0 | A0A3L7I0Q0_CRIGR | Carboxylic | CgPICR_005597 | Cricetulus griseus | 627 |
| | | ester | | (Chinese hamster) | |
| | | hydrolase | | (Cricetulus | |
| | | | | barabensis griseus) | |
| A0A3L710P0 | A0A3L7I0P0_CRIGR | Carboxylic | CgPICR_005587 | Cricetulus griseus | 548 |
| | | ester | | (Chinese hamster) | |
| | | hydrolase | | (Cricetulus | |
| | | | | barabensis griseus) | |
| A0A3L7HYP5 | A0A3L7HYP5_CRIGR | Abhydrolipase | CgPICR_006572 | Cricetulus griseus | 210 |
| | | domain- | | (Chinese hamster) | |
| | | containing | | (Cricetulus | |
| | | protein | | barabensis griseus) | |
| A0A061HUZ2 | A0A061HUZ2_CRIGR | Platelet- | H671_21690 | Cricetulus griseus | 231 |
| | | activating | | (Chinese hamster) | |
| | | factor | | (Cricetulus | |
| | | acetylhy . . . | | barabensis griseus) | |
| A0A3L7I017 | A0A3L7I017_CRIGR | LCAT | CgPICR_005642 | Cricetulus griseus | 458 |
| | | | | (Chinese hamster) | |
| | | | | (Cricetulus | |
| | | | | barabensis griseus) | |
| G3I9K8 | G3I9K8_CRIGR | Arylacetamid | I79_020257 | Cricetulus griseus | 363 |
| | | deacetylase- | | (Chinese hamster) | |
| | | like 2 | | (Cricetulus | |
| | | | | barabensis griseus) | |
| A0A3L7IIN6 | A0A3L7IIN6_CRIGR | AADACL2 | CgPICR_009977 | Cricetulus griseus | 401 |
| | | | | (Chinese hamster) | |
| | | | | (Cricetulus | |
| | | | | barabensis griseus) | |
| G3HQX9 | G3HQX9_CRIGR | Lipase | I79_013238 | Cricetulus griseus | 484 |
| | | member M | | (Chinese hamster) | |
| | | | | (Cricetulus | |
| | | | | barabensis griseus) | |
| G3HQX2 | G3HQX2_CRIGR | Gastric | I79_013231 | Cricetulus griseus | 185 |
| | | triacylglycerol | | (Chinese hamster) | |
| | | lipase | | (Cricetulus | |
| | | | | barabensis griseus) | |
| A0A3L7I2M8 | A0A3L7I2M8_CRIGR | Carboxylic | CgPICR_005589 | Cricetulus griseus | 498 |
| | | ester | | (Chinese hamster) | |
| | | hydrolase | | (Cricetulus | |
| | | | | barabensis griseus) | |
| G3GVB7 | G3GVB7_CRIGR | Sn1-specific | I79_001652 | Cricetulus griseus | 154 |
| | | diacylglycerol | | (Chinese hamster) | |
| | | lipase . . . | | (Cricetulus | |
| | | | | barabensis griseus) | |
| G3I0M0 | G3I0M0_CRIGR | Hormone- | I79_016908 | Cricetulus griseus | 326 |
| | | sensitive | | (Chinese hamster) | |
| | | lipase | | (Cricetulus | |
| | | | | barabensis griseus) | |
| A0A061I5N2 | A0A061I5N2_CRIGR | DUF676 | H671_4g12871 | Cricetulus griseus | 1,082 |
| | | domain- | | (Chinese hamster) | |
| | | containing | | (Cricetulus | |
| | | protein | | barabensis griseus) | |
| A0A061IQA8 | A0A061IQA8_CRIGR | DUF676 | H671_1g1584 | Cricetulus griseus | 1,609 |
| | | domain- | | (Chinese hamster) | |
| | | containing | | (Cricetulus | |
| | | protein | | barabensis griseus) | |
| A0A061I6F9 | A0A061I6F9_CRIGR | DUF676 | H671_4g12871 | Cricetulus griseus | 1,172 |
| | | domain- | | (Chinese hamster) | |
| | | containing | | (Cricetulus | |
| | | protein | | barabensis griseus) | |
| A0A3L7HYS2 | A0A3L7HYS2_CRIGR | AB | CgPICR_006573 | Cricetulus griseus | 449 |
| | | hydrolase-1 | | (Chinese hamster) | |
| | | domain- | | (Cricetulus | |
| | | containing | | barabensis griseus) | |
| | | protein | | | |
| A0A061IEN9 | A0A061IEN9_CRIGR | Patatin-like | H671_3g9878 | Cricetulus griseus | 429 |
| | | phospholipase | | (Chinese hamster) | |
| | | domain-c . . . | | (Cricetulus | |
| | | | | barabensis griseus) | |
| A0A061I428 | A0A061I428_CRIGR | Group XV | H671_4g12540 | Cricetulus griseus | 201 |
| | | phospholipase | | (Chinese hamster) | |
| | | A2-like | | (Cricetulus | |
| | | protein | | barabensis griseus) | |
| G3I769 | G31769_CRIGR | Carboxylic | I79_019354 | Cricetulus griseus | 545 |
| | | ester | | (Chinese hamster) | |
| | | hydrolase | | (Cricetulus | |
| | | | | barabensis griseus) | |
| G3HZJ6 | G3HZJ6_CRIGR | Carboxylic | I79_016502 | Cricetulus griseus | 240 |
| | | ester | | (Chinese hamster) | |
| | | hydrolase | | (Cricetulus | |
| | | | | barabensis griseus) | |
| G3IIG3 | G3IIG3_CRIGR | Carboxylic | I79_023632 | Cricetulus griseus | 511 |
| | | ester | | (Chinese hamster) | |
| | | hydrolase | | (Cricetulus | |
| | | | | barabensis griseus) | |
| G3HZJ8 | G3HZJ8_CRIGR | Carboxylic | I79_016504 | Cricetulus griseus | 393 |
| | | ester | | (Chinese hamster) | |
| | | hydrolase | | (Cricetulus | |
| | | | | barabensis griseus) | |
| G3I7X4 | G3I7X4_CRIGR | Carboxylic | I79_019625 | Cricetulus griseus | 514 |
| | | ester | | (Chinese hamster) | |
| | | hydrolase | | (Cricetulus | |
| | | | | barabensis griseus) | |
| G3I765 | G3I765_CRIGR | Carboxylic | I79_019350 | Cricetulus griseus | 128 |
| | | ester | | (Chinese hamster) | |
| | | hydrolase | | (Cricetulus | |
| | | | | barabensis griseus) | |
| A0A061IDX0 | A0A061IDX0_CRIGR | Carboxylic | H671_3g10207 | Cricetulus griseus | 554 |
| | | ester | | (Chinese hamster) | |
| | | hydrolase | | (Cricetulus | |
| | | | | barabensis griseus) | |
| A0A061IGP0 | A0A061IGP0_CRIGR | Carboxylic | H671_3g9072 | Cricetulus griseus | 545 |
| | | ester | | (Chinese hamster) | |
| | | hydrolase | | (Cricetulus | |
| | | | | barabensis griseus) | |
| A0A061IEJ6 | A0A061IEJ6_CRIGR | Carboxylic | H671_3g9076 | Cricetulus griseus | 543 |
| | | ester | | (Chinese hamster) | |
| | | hydrolase | | (Cricetulus | |
| | | | | barabensis griseus) | |
| A0A061IDC4 | A0A061IDC4_CRIGR | Carboxylic | H671_3g10598 | Cricetulus griseus | 531 |
| | | ester | | (Chinese hamster) | |
| | | hydrolase | | (Cricetulus | |
| | | | | barabensis griseus) | |
| A0A061IAS9 | A0A061IAS9_CR1GR | Carboxylic | H671_3g10207 | Cricetulus griseus | 466 |
| | | ester | | (Chinese hamster) | |
| | | hydrolase | | (Cricetulus | |
| | | | | barabensis griseus) | |
| A0A061IBR3 | A0A061IBR3_CRIGR | Carboxylic | H671_3g10207 | Cricetulus griseus | 541 |
| | | ester | | (Chinese hamster) | |
| | | hydrolase | | (Cricetulus | |
| | | | | barabensis griseus) | |
| G3I770 | G3I770_CRIGR | Carboxylic | I79_019355 | Cricetulus griseus | 420 |
| | | ester | | (Chinese hamster) | |
| | | hydrolase | | (Cricetulus | |
| | | | | barabensis griseus) | |
| G3I773 | G3I773_CRIGR | Carboxylic | I79_019358 | Cricetulus griseus | 393 |
| | | ester | | (Chinese hamster) | |
| | | hydrolase | | (Cricetulus | |
| | | | | barabensis griseus) | |
| A0A3L7I051 | A0A3L7I051_CRIGR | Carboxylic | CgPICR_005600 | Cricetulus griseus | 558 |
| | | ester | | (Chinese hamster) | |
| | | hydrolase | | (Cricetulus | |
| | | | | barabensis griseus) | |
| A0A3L7I1A1 | A0A3L7I1A1_CRIGR | Carboxylic | CgPICR_005488 | Cricetulus griseus | 584 |
| | | ester | | (Chinese hamster) | |
| | | hydrolase | | (Cricetulus | |
| | | | | barabensis griseus) | |
| G3HII7 | G3HII7_CRIGR | Carboxylic | I79_010458 | Cricetulus griseus | 451 |
| | | ester | | (Chinese hamster) | |
| | | hydrolase | | (Cricetulus | |
| | | | | barabensis griseus) | |
| G3I774 | G3I774_CRIGR | Carboxylic | I79_019359 | Cricetulus griseus | 352 |
| | | ester | | (Chinese hamster) | |
| | | hydrolase | | (Cricetulus | |
| | | | | barabensis griseus) | |
| G3I5K9 | G3I5K9_CRIGR | Carboxylic | I79_018762 | Cricetulus griseus | 370 |
| | | ester | | (Chinese hamster) | |
| | | hydrolase | | (Cricetulus | |
| | | | | barabensis griseus) | |
| A0A3L7HLV1 | A0A3L7HLV1_CRIGR | ACHE | CgPICR_017300 | Cricetulus griseus | 929 |
| | | | | (Chinese hamster) | |
| | | | | (Cricetulus | |
| | | | | barabensis griseus) | |
| A0A3L7I019 | A0A3L7I019_CRIGR | Carboxylic | CgPICR_005594 | Cricetulus griseus | 535 |
| | | ester | | (Chinese hamster) | |
| | | hydrolase | | (Cricetulus | |
| | | | | barabensis griseus) | |
| A0A061I790 | A0A061I790_CRIGR | Carboxylic | H671_3g10206 | Cricetulus griseus | 496 |
| | | ester | | (Chinese hamster) | |
| | | hydrolase | | (Cricetulus | |
| | | | | barabensis griseus) | |
| A0A061IDA6 | A0A061IDA6_CRIGR | Carboxylic | H671_3g10649 | Cricetulus griseus | 352 |
| | | ester | | (Chinese hamster) | |
| | | hydrolase | | (Cricetulus | |
| | | | | barabensis griseus) | |
| G3I5K8 | G3I5K8_CRIGR | Carboxylic | I79_018761 | Cricetulus griseus | 448 |
| | | ester | | (Chinese hamster) | |
| | | hydrolase | | (Cricetulus | |
| | | | | barabensis griseus) | |
| A0A3L7HZX1 | A0A3L7HZX1_CRIGR | Carboxylic | CgPICR_005595 | Cricetulus griseus | 560 |
| | | ester | | (Chinese hamster) | |
| | | hydrolase | | (Cricetulus | |
| | | | | barabensis griseus) | |
| G3HII8 | G3HII8_CRIGR | Carboxylic | I79_010459 | Cricetulus griseus | 411 |
| | | ester | | (Chinese hamster) | |
| | | hydrolase | | (Cricetulus | |
| | | | | barabensis griseus) | |
| G3I768 | G3I768_CRIGR | Carboxylic | I79_019353 | Cricetulus griseus | 449 |
| | | ester | | (Chinese hamster) | |
| | | hydrolase | | (Cricetulus | |
| | | | | barabensis griseus) | |
| A0A3L7HZQ0 | A0A3L7HZQ0_CRIGR | Carboxylic | CgPICR_005491 | Cricetulus griseus | 569 |
| | | ester | | (Chinese hamster) | |
| | | hydrolase | | (Cricetulus | |
| | | | | barabensis griseus) | |
| A0A3L7HZV5 | A0A3L7HZV5_CRIGR | Carboxylic | CgPICR_005500 | Cricetulus griseus | 529 |
| | | ester | | (Chinese hamster) | |
| | | hydrolase | | (Cricetulus | |
| | | | | barabensis griseus) | |
| A0A3L7I1I7 | A0A3L7I1I7_CRIGR | Carboxylic | CgPICR_005588 | Cricetulus griseus | 611 |
| | | ester | | (Chinese hamster) | |
| | | hydrolase | | (Cricetulus | |
| | | | | barabensis griseus) | |
| A0A3L7HZU5 | A0A3L7HZU5_CRIGR | Carboxylic | CgPICR_005490 | Cricetulus griseus | 304 |
| | | ester | | (Chinese hamster) | |
| | | hydrolase | | (Cricetulus | |
| | | | | barabensis griseus) | |
| A0A061IGP5 | A0A061IGP5_CRIGR | Carboxylic | H671_3g9077 | Cricetulus griseus | 398 |
| | | ester | | (Chinese hamster) | |
| | | hydrolase | | (Cricetulus | |
| | | | | barabensis griseus) | |
| A0A3L7HZM4 | A0A3L7HZM4_CRIGR | Carboxylic | CgPICR_005495 | Cricetulus griseus | 419 |
| | | ester | | (Chinese hamster) | |
| | | hydrolase | | (Cricetulus | |
| | | | | barabensis griseus) | |
| G3HFM0 | G3HFM0_CRIGR | Monoacylglycerol | I79_009382 | Cricetulus griseus | 330 |
| | | lipase | | (Chinese hamster) | |
| | | ABHD6 | | (Cricetulus | |
| | | | | barabensis griseus) | |
| G3HSU0 | G3HSU0_CRIGR | Protein | I79_013937 | Cricetulus griseus | 1,174 |
| | | FAM135B | | (Chinese hamster) | |
| | | | | (Cricetulus | |
| | | | | barabensis griseus) | |
| A0A061IFE2 | A0A061IFE2_CRIGR | Liver | H671_3g9583 | Cricetulus griseus | 882 |
| | | carboxylesterase | | (Chinese hamster) | |
| | | 1-like | | (Cricetulus | |
| | | protein | | barabensis griseus) | |
| A0A061I6Q8 | A0A061I6Q8_CRIGR | Liver | H671_3g10564 | Cricetulus griseus | 820 |
| | | carboxylesterase | | (Chinese hamster) | |
| | | B-1-like | | (Cricetulus | |
| | | protein | | barabensis griseus) | |
| A0A061IAA7 | A0A061IAA7_CRIGR | Liver | H671_3g10564 | Cricetulus griseus | 704 |
| | | carboxylesterase | | (Chinese hamster) | |
| | | B-1-like | | (Cricetulus | |
| | | protein | | barabensis griseus) | |
| A0A061I7X9 | A0A061I7X9_CRIGR | Liver | H671_3g10564 | Cricetulus griseus | 704 |
| | | carboxylesterase | | (Chinese hamster) | |
| | | B-1-like | | (Cricetulus | |
| | | protein | | barabensis griseus) | |
| A0A3L7HRB9 | A0A3L7HRB9_CRIGR | LIPH | CgPICR_002185 | Cricetulus griseus | 196 |
| | | | | (Chinese hamster) | |
| | | | | (Cricetulus | |
| | | | | barabensis griseus) | |
| A0A3L7IHZ7 | A0A3L7IHZ7_CRIGR | AADACL2 | CgPICR_009975 | Cricetulus griseus | 401 |
| | | | | (Chinese hamster) | |
| | | | | (Cricetulus | |
| | | | | barabensis griseus) | |
| G3GW90 | G3GW90_CRIGR | Phospholipase B1, | I79_002010 | Cricetulus griseus | 995 |
| | | membrane- | | (Chinese hamster) | |
| | | associat . . . | | (Cricetulus | |
| | | | | barabensis griseus) | |
| G3I9K7 | G3I9K7_CRIGR | Arylaceiamide | I79_020256 | Cricetulus griseus | 392 |
| | | deacetylase | | (Chinese hamster) | |
| | | | | (Cricetulus | |
| | | | | barabensis griseus) | |
| A0A061I741 | A0A061I741_CRIGR | Liver | H671_3g11295 | Cricetulus griseus | 981 |
| | | carboxylesterase- | | (Chinese hamster) | |
| | | like | | (Cricetulus | |
| | | protein | | barabensis griseus) | |
| A0A061ID92 | A0A061ID92_CRIGR | Liver | H671_3g9582 | Cricetulus griseus | 905 |
| | | carboxylesterase | | (Chinese hamster) | |
| | | 1-like | | (Cricetulus | |
| | | protein | | barabensis griseus) | |
| G3GUE1 | G3GUE1_CRIGR | Protein | I79_001298 | Cricetulus griseus | 1,546 |
| | | FAM135A | | (Chinese hamster) | |
| | | | | (Cricetulus | |
| | | | | barabensis griseus) | |
| A0A3L7HRF9 | A0A3L7HRF9_CRIGR | LIPH | CgPICR_002186 | Cricetulus griseus | 208 |
| | | | | (Chinese hamster) | |
| | | | | (Cricetulus | |
| | | | | barabensis griseus) | |
| G3IM61 | G3IM61_CRIGR | Thyroglobulin | I79_024992 | Cricetulus griseus | 241 |
| | | | | (Chinese hamster) | |
| | | | | (Cricetulus | |
| | | | | barabensis griseus) | |
| G3IHH9 | G3IHH9_CRIGR | Isoamyl | I79_023269 | Cricetulus griseus | 280 |
| | | acetate- | | (Chinese hamster) | |
| | | hydrolyzing | | (Cricetulus | |
| | | esteras . . . | | barabensis griseus) | |
| A0A3L7IHB1 | A0A3L7IHB1_CRIGR | NCEH1 | CgPICR_002419 | Cricetulus griseus | 237 |
| | | | | (Chinese hamster) | |
| | | | | (Cricetulus | |
| | | | | barabensis griseus) | |
| A0A3L7HP21 | A0A3L7HP21_CRIGR | FAM135B | CgPICR_014371 | Cricetulus griseus | 1,270 |
| | | | | (Chinese hamster) | |
| | | | | (Cricetulus | |
| | | | | barabensis griseus) | |
| A0A3L7H5L4 | A0A3L7H5L4_CRIGR | LIPE | CgPICR_018531 | Cricetulus griseus | 444 |
| | | | | (Chinese hamster) | |
| | | | | (Cricetulus | |
| | | | | barabensis griseus) | |
| A0A3L7HQ89 | A0A3L7HQ89_CRIGR | DAGLB | CgPICR_011708 | Cricetulus griseus | 655 |
| | | | | (Chinese hamster) | |
| | | | | (Cricetulus | |
| | | | | barabensis griseus) | |
| G3HKW5 | G3HKW5_CRIGR | Phosphatidyl | I79_011347 | Cricetulus griseus | 183 |
| | | choline- | | (Chinese hamster) | |
| | | sterol | | (Cricetulus | |
| | | acyltran . . . | | barabensis griseus) | |
| A0A3L7HZQ8 | A0A3L7HZQ8_CRIGR | CES1D | CgPICR_005484 | Cricetulus griseus | 1,211 |
| | | | | (Chinese hamster) | |
| | | | | (Cricetulus | |
| | | | | barabensis griseus) | |
| A0A3L7HYL8 | A0A3L7HYL8_CRIGR | LIPK | CgPICR_006564 | Cricetulus griseus | 223 |
| | | | | (Chinese hamster) | |
| | | | | (Cricetulus | |
| | | | | barabensis griseus) | |
| G3HQX7 | G3HQX7_CRIGR | Gastric | I79_013235 | Cricetulus griseus | 151 |
| | | triacylglycerol | | (Chinese hamster) | |
| | | lipase | | (Cricetulus | |
| | | | | barabensis griseus) | |
| G3HQX6 | G3HQX6_CRIGR | Gastric | I79_013236 | Cricetulus griseus | 252 |
| | | triacylglycerol | | (Chinese hamster) | |
| | | lipase | | (Cricetulus | |
| | | | | barabensis griseus) | |
| A0A061HVA0 | A0A061HVA0_CRIGR | Patatin-like | H671_xg19912 | Cricetulus griseus | 58 |
| | | phospholipase | | (Chinese hamster) | |
| | | domain-c . . . | | (Cricetulus | |
| | | | | barabensis griseus) | |
| A0A3L7IGW9 | A0A3L7IGW9_CRIGR | FAM135A | CgPICR_013396 | Cricetulus griseus | 1,183 |
| | | | | (Chinese hamster) | |
| | | | | (Cricetulus | |
| | | | | barabensis griseus) | |
| G3I0M2 | G310M2_CRIGR | Platelet- | I79_016910 | Cricetulus griseus | 228 |
| | | activating | | (Chinese hamster) | |
| | | factor | | (Cricetulus | |
| | | acetylhy . . . | | barabensis griseus) | |
| A0A3L7HBC2 | A0A3L7HBC2_CRIGR | IAH1 | CgPICR_006476 | Cricetulus griseus | 245 |
| | | | | (Chinese hamster) | |
| | | | | (Cricetulus | |
| | | | | barabensis griseus) | |
| A0A3L7HT19 | A0A3L7HT19_CRIGR | PAFAH1B2 | CgPICR_005977 | Cricetulus griseus | 214 |
| | | | | (Chinese hamster) | |
| | | | | (Cricetulus | |
| | | | | barabensis griseus) | |
| G3I767 | G3I767_CRIGR | Liver | I79_019352 | Cricetulus griseus | 795 |
| | | carboxylesterase | | (Chinese hamster) | |
| | | | | (Cricetulus | |
| | | | | barabensis griseus) | |
| G3I7X9 | G3I7X9_CRIGR | Liver | I79_019630 | Cricetulus griseus | 765 |
| | | carboxylesterase 1 | | (Chinese hamster) | |
| | | | | (Cricetulus | |
| | | | | barabensis griseus) | |
| A0A3L7I0E5 | A0A3L7I0E5_CRIGR | CES1 | CgPICR_005497 | Cricetulus griseus | 1,118 |
| | | | | (Chinese hamster) | |
| | | | | (Cricetulus | |
| | | | | barabensis griseus) | |
| A0A3L7I003 | A0A3L7I003_CRIGR | CES2C | CgPICR_005601 | Cricetulus griseus | 825 |
| | | | | (Chinese hamster) | |
| | | | | (Cricetulus | |
| | | | | barabensis griseus) | |
| G3H7V6 | G3H7V6_CRIGR | Monoglyceride | I79_006450 | Cricetulus griseus | 328 |
| | | lipase | | (Chinese hamster) | |
| | | | | (Cricetulus | |
| | | | | barabensis griseus) | |
| G3HQX4 | G3HQX4_CRIGR | Lipase | I79_013233 | Cricetulus griseus | 159 |
| | | member K | | (Chinese hamster) | |
| | | | | (Cricetulus | |
| | | | | barabensis griseus) | |
| G3GVB6 | G3GVB6_CRIGR | Sn1-specific | I79_001651 | Cricetulus griseus | 167 |
| | | diacylglycerol | | (Chinese hamster) | |
| | | lipase . . . | | (Cricetulus | |
| | | | | barabensis griseus) | |
| A0A3L71GK4 | A0A3L7IGK4_CRIGR | FAM135A | CgPICR_013395 | Cricetulus griseus | 320 |
| | | | | (Chinese hamster) | |
| | | | | (Cricetulus | |
| | | | | barabensis griseus) | |

The lipases of the table above may be found in CHO cells, for example.

Lipases represent a class of HCPs that have been identified in discovery experiments, and are believed to play an important role in formulation shelf-life. Lipases, in the class of esterases, have the capacity to act on Polysorbate 20 (PS20), leading to the generation of degradants that may form solid particles.

Of the lipases identified, phospholipase B like protein (PLBL2) has been best characterized. While shown to bind to antibody drug products, it is no longer thought to play a significant role in PS20 degradation. Yet other members of the class, found with lower abundance, represent targets of interest for characterization.

The disclosure also contemplates modifications of host cells, for example, to reduce protein-lipase, e.g., antibody-lipase interactions, as well as modifications in cell culture conditions to reduce such interactions, and methods of producing a protein or an antibody in such cells or conditions, optionally including a step of determining protein-lipase or antibody-lipase interactions. In some embodiments, the disclosure includes a mammalian host cell for expression of a recombinant protein, where the cell is modified to alter expression of one or more endogenous enzymes involved in glycosylation of an endogenous lipase relative to the expression of the endogenous enzymes in an unmodified cell. Examples include mutations, down-regulation, or knock-outs of enzymes, such as alpha-Man-1 and 2, to prevent processing of the Asn-linked Man₉GlcNac₂ glycan precursor and/or increase high MW high mannose species; metabolic approaches resulting in higher chain length glycans, such as modulation of feed sources to increase proportion of raffinose, monensin, mannose, galactose, fructose, and maltose; the addition of high mannose promoting inhibitors. e.g., kifunensine; and/or over-expression enzymes to upregulate to increase the chain length of glycans, such as GNT-1, 2, 3, 4abc, 5, and GalT. In some embodiments, the host cell is modified to reduce the relative amount of low-number mannose glycans in endogenous lipases. For example, as described in the Examples below, lipases that more tightly bound antibodies were enriched in low-number mannose glycans such as Man3-5, or Man6 or smaller. Lipases that were less prone to bind antibodies tended to have mannosylated species of greater than 1200 Daltons, or Man7-9. Thus, in some embodiments, host cells are modified to produce lipases with mannosylated glycosylation modifications that are of relatively higher molecular weight, such as >1200 Da, or to produce mannosylated glycosylation modifications with relatively higher levels of Man6 or higher, or Man7 or higher, or Man7-9 compared to Man3-5. (See, e.g., Clausen et al., Glycosylation Engineering—in Essentials of Glycobiology. Varki, et al., Eds., Cold Spring Harbor Laboratory Press, doi: 10.1101/glycobiology.3e.056 (2015-2017).)

In one embodiment, the invention includes a mammalian cell comprising one or more endogenous lipases that have been mutated (substitution, deletion, or addition) in at least one amino acid, and where the modification results in altered interaction with a protein or antibody. For example, endogenous lipases may be mutated to have reduced or eliminated glycosylation. Glycosylation of lipases may occur at an N-linked glycosylation motif “N-X-S/T” in which the first amino acid residue is N, the third amino acid residue is either S or T, and the second amino acid residue (X) is any amino acid other than proline (P). In one example, the wild-type (WT) Asn residue that corresponds to the location of N-linked glycosylation (motif N-X-S/T) is mutated to any other amino acid. In another example, the second amino acid residue could be mutated to P. In another example, the third amino acid residue (S or T) could be mutated to a different residue. Or, in some cases, more than one of these substitutions could be made. Specific examples of lipases amenable to such mutations include any of those listed above and in the above table, such as, for example. Thioesterase, Lipoprotein-associated Phospholipase A2, Phospholipase B-Like 2, palmitoyl protein thioesterase, phospholipase D3, and sphingomyelin phosphodiesterase, e.g., as presented above. Locations of these N-X-S/T motifs are shown in SEQ ID Nos: 1-6 for particular lipases found in Chinese hamster ovary (CHO) cells above each one of the sequences above. In some cases, the host cell is a CHO cell. For example, thioesterase has an N-X-S/T site at positions 298-300 and 422-424; LPLA2 has such sites at positions 99-101, 273-275, 289-291, and 398-401; PLBL2 at 47-49, 65-67, 69-71, 190-192, 395-397, and 474-476; PPT at 197-199, 212-214, and 232-234; PLD3 at 97-99, 102-104, 132-134, 234-236, 282-284, 385-387, and 430-432; and SP at 84-86, 173-175, 333-335, 393-395, 518-520, and 611-613 (see SEQ ID Nos: 1-6, respectively, shown above with the N residues underlined in each case).

In other cases, the N-linked glycosylation motif may be “N-X-C”, in which X is any residue except proline. For such an N-linked glycosylation site in a lipase, the N or C may be modified to a different amino acid, and/or the X may be modified to proline, for example.

Accordingly, embodiments of the disclosure include a modified host cell, such as a CHO cell, with an amino acid substitution at one or more of the above amino acid residues in PLBL2 and/or LPLA2, or another of the above lipases, as well as methods of producing antibodies from such a cell, including optionally determining the level of lipase-antibody interaction following production of the antibodies.

The present disclosure also contemplates methods of producing proteins or antibodies with reduced lipase interactions by altering the cell culture conditions of the host cell, for example. Culture conditions that may enhance the levels of mannosylated glycosylation modifications of Man6 or higher, or Man7 or higher, or Man7-9 compared to Man3-5, for example, may be used. See, for example. Pacis et al., Biotechnology and Bioengineering, 108(10): 2348-58 (2011); Rameez et al., Biotechnology Progress, 37(5): e3176, DOI: 10.1002/ptpr.3176 (2021). Examples include increasing osmolality of the culture medium, for example, by at least 100 or at least 200 mOsm/kg, adding manganese chloride or ammonium chloride to the medium, or altering pH or sugar and amino acid concentrations. Additional examples include increasing or adding raffinose, monensin, mannose, galactose, fructose, and/or maltose, as well as adding high mannose promoting inhibitors such as Kifunensine to the medium. In some cases, the change in cell culture conditions results in a significant increase in the percentage Man7-9 as compared to overall mannosylated species. In some cases, the change in cell culture conditions results in an overall Mann7-9 percentage as compared to overall mannosylated species of, for example, at least 15% or at least 20%.

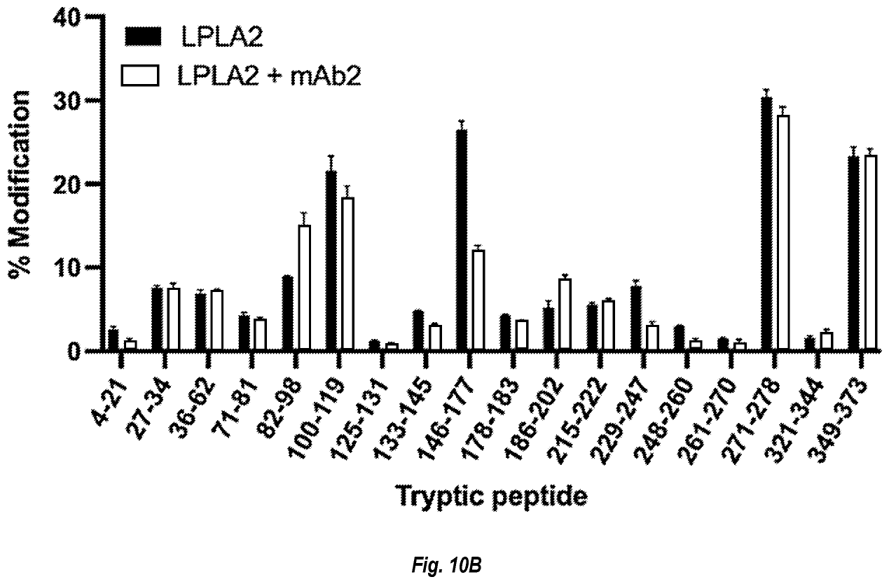

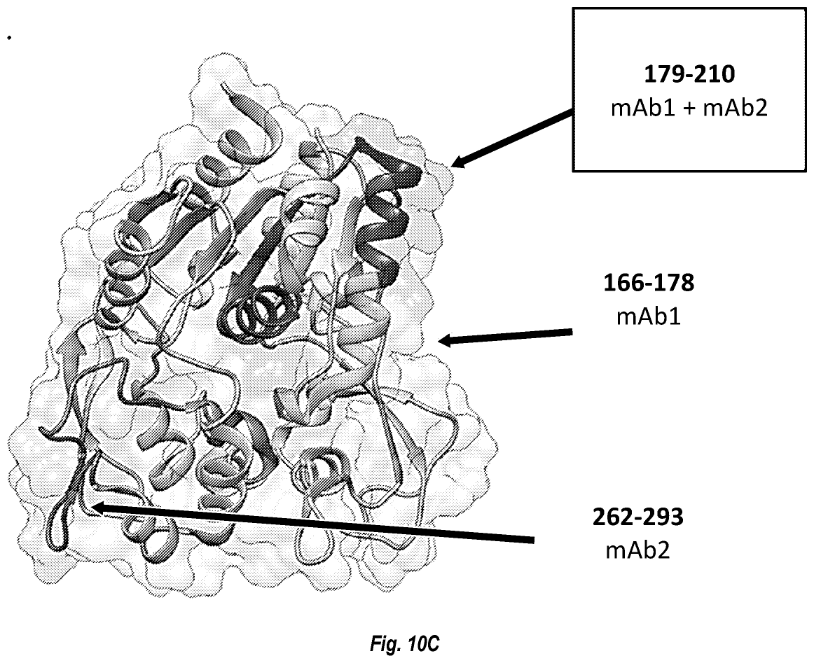

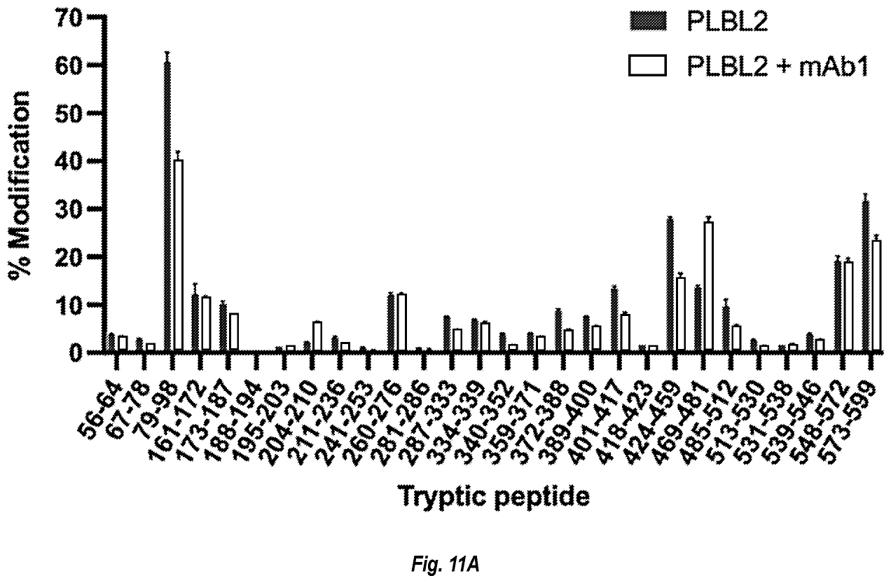

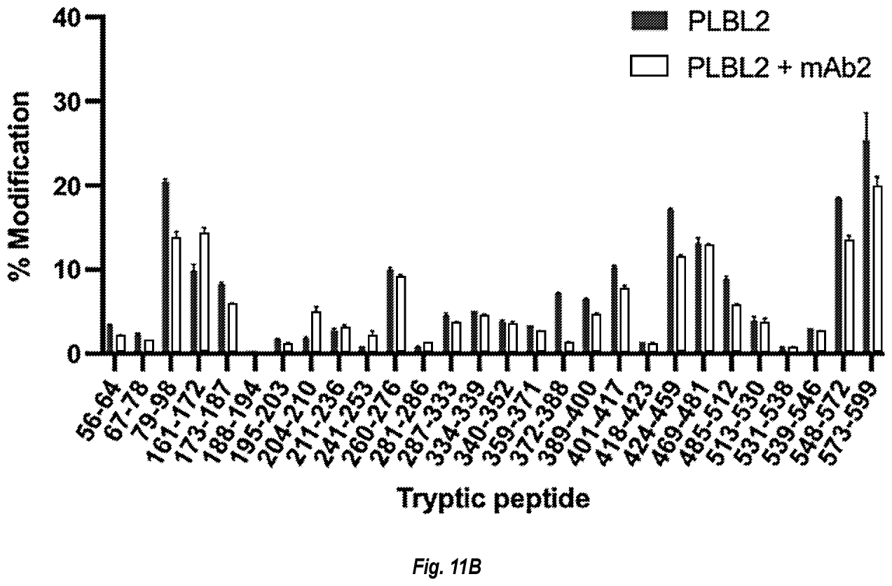

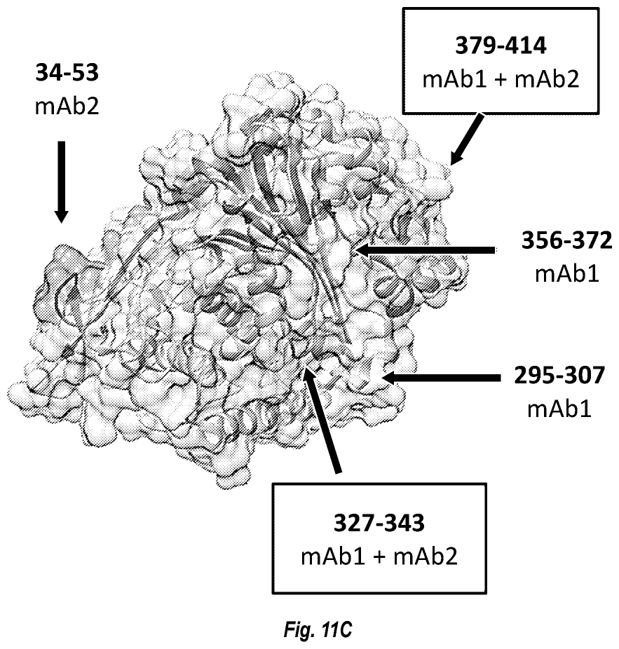

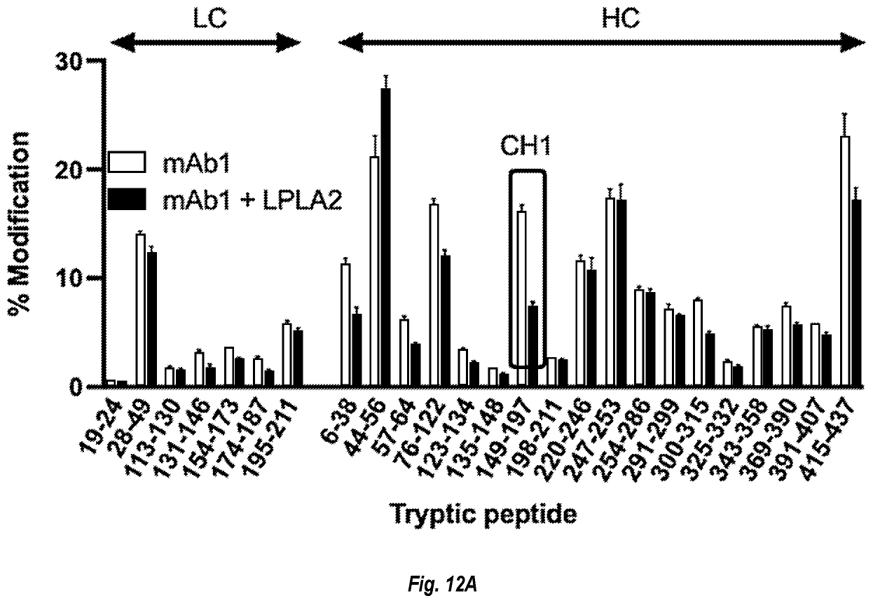

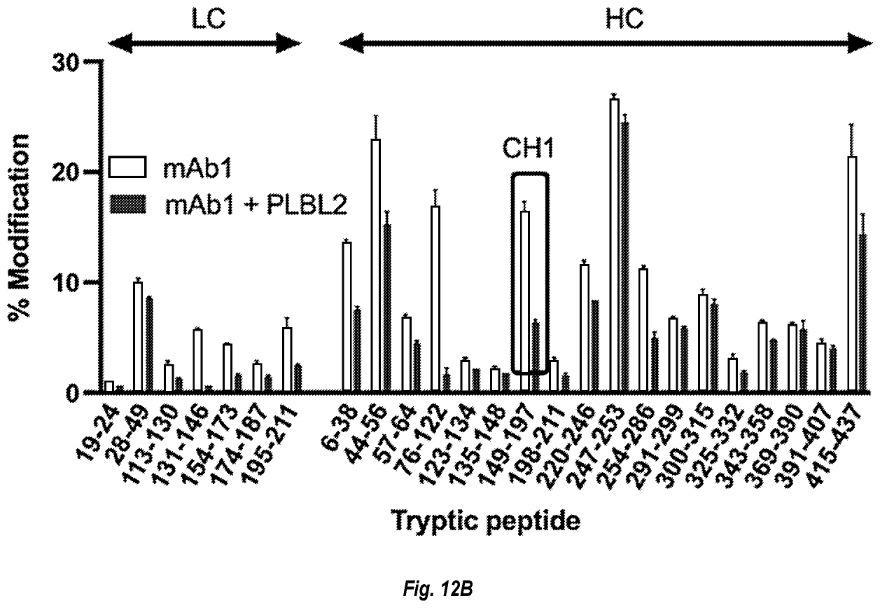

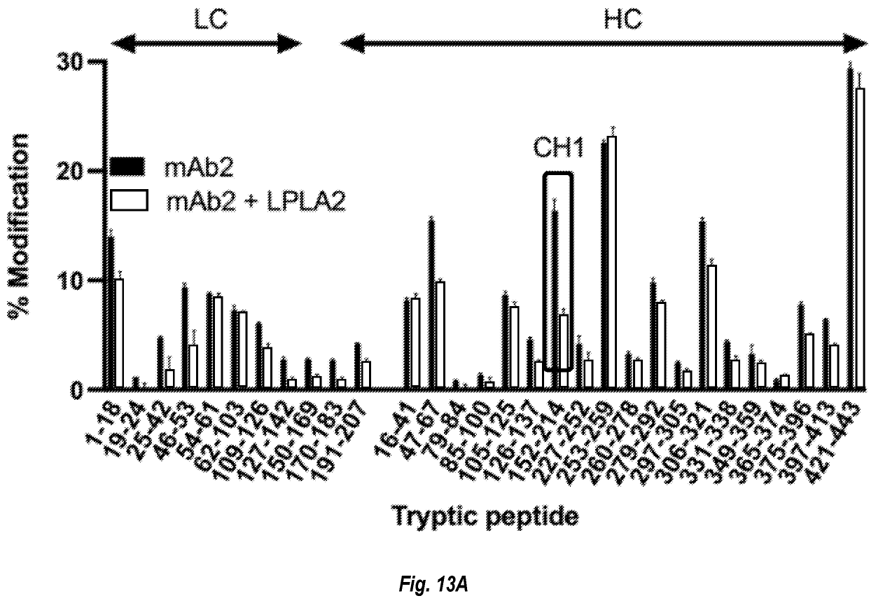

In addition, as shown in the Examples, peptides 125-131, 133-135 and 146-177 of LPLA2 displayed a significant decrease in oxidation when complexed with a monoclonal antibody mAb1 (FIG. 10A). By way of another example, LPLA2 incubated with an excess of a monoclonal antibody mAb2, peptides 133-135, 146-177, 229-247 and 248-260 displayed reduced oxidation indicating involvement in binding to mAb2 (FIG. 10B). In particular, peptide 146-177 was a common interacting region for both antibodies. (See Example 1 below.) Similarly, peptides 67-78, 79-98, 173-187, 211-236, 241-253, 287-333, 340-352, 359-371, 372-388, 389-400, 401-407, 424-459, 513-530, 539-546 and 573-599 of PLBL2 displayed reduced oxidation in the PLBL2-mAb1 complex and peptides 56-64, 67-78, 79-98, 173-187, 359-371, 372-388, 389-40), 401-407, 424-459, 485-512 and 548-572 displayed reduced oxidation in the PLBL2-mAb2 complex (FIG. 11A-B). Peptide 379-414 was identified as the common binding epitope on PLBL2 for both mAb1 and mAb2 (FIG. 11C). Peptides 79-98, 424-459 and 573-599 were common interacting regions with both antibodies. (See Example 1 below.) Mutations in one of these lipase regions could also be made as a means of modifying a host cell, such as a CHO cell, for reduced interaction between antibodies and the endogenous lipases PLBL2 and LPLA2. Accordingly, further contemplated herein arc methods of determining particular regions of an endogenous host cell lipase that display reduced oxidation levels, according to methods herein, indicating contact with an antibody product, and preparing amino acid substitutions in at least one amino acid within those regions.

### Methods of Detecting Lipase-Antibody Binding and Determining Binding Affinity

Host cell proteins (HCP) that are co-purified in antibody formulations, such as lipases, are traditionally identified through precipitation and enrichment experiments. Validation may be performed by over-expressing the proteins of interest, and directly assessing if the target binds. However, HCPs of interest may be <1% of the total proteins identified, making discovery difficult.

Techniques that rely on immobilization (for example surface plasmon resonance), look at proteins in a non-native state. Likewise, conjugation assays may alter the behavior of protein. Native mass spectrometry biophysical assays (native MS, HDX, FPOP) represent the gold standard for analysis.

The following work uses multiple techniques to establish the binding of several lipases across different antibodies. Through that analysis, structural regions of interest on both the antibody and lipase, important to mediating binding, were revealed. The work demonstrates characterization of antibody-lipases by native mass spectrometry and non-MS ion mobility for the first time, allowing stoichiometry and structure to be examined in unique ways.

Accordingly, in some embodiments, binding between antibodies and at least one lipase derived from a host cell is detected, optionally with binding affinity also being measured. In some cases, the level of binding and/or the affinity is compared to that of an antibody that is not modified to alter lipase interactions, but that otherwise is structurally identical. (i.e., where the antibody heavy chain constant region comprises an amino acid modification, binding is compared to an antibody that lacks the modification but otherwise has the same amino acid sequence, or where the antibody has a modified glycosylation state, binding is compared to an antibody that lacks the glycosylation modification but that otherwise does not differ). In some cases, binding is detected and/or affinity determined by surface plasmon resonance (SPR), microscale thermophoresis (MST), and/or ELISA, for example, with a purified lipase protein in vitro. In some cases, binding is detected by SPR, hydroxyl radical footprinting, native mass spectrometry, and/or ion mobility assays.

### Exemplary Formulations and Excipients

The disclosure herein also relates to formulations comprising the antibodies herein, which may, in some embodiments be for therapeutic use. Formulations herein may comprise at least one excipient, such as one or more of a pharmaceutically acceptable acid or base, buffers, salts, lyoprotectant (if the formulation is to be lyophilized), sugar, sugar alcohol, amino acid, an additional protein species, diluents, preservatives, polyvalent metal salts, and, in some cases a surfactant. In some cases, formulations may comprise a surfactant such as a polysorbate, poloxamer, pluronic, Brij, or alkylglycoside surfactant. Examples of surfactants include poly sorbates, such as polysorbate 20 (PS20) and polysorbate 80 (PS80). Other additional surfactants may include poloxamers and pluronics, such as poloxamer 188 or pluronic F68, or Brij. Other additional surfactants may include alkylglycosides, such as octyl maltoside, decyl maltoside, dodecyl maltoside, or octyl glucoside.

A “stabilizer” herein means any added excipient that is added to a formulation to help maintain it in a stable or unchanging state. In some cases, a stabilizer may be added to help prevent aggregation, oxidation, color changes, or the like.

A “pharmaceutically acceptable acid” includes inorganic and organic acids which are non-toxic at the concentration and manner in which they are formulated. For example, suitable inorganic acids include hydrochloric, perchloric, hydrobromic, hydroiodic, nitric, sulfuric, sulfonic, sulfnic, sulfanilic, phosphoric, carbonic, etc. Suitable organic acids include straight and branched-chain alkyl, aromatic, cyclic, cycloaliphatic, arylaliphatic, heterocyclic, saturated, unsaturated, mono, di- and tri-carboxylic, including for example, formic, acetic, 2-hydroxyacetic, trifluoroacetic, phenylacetic, trimethylacetic, t butyl acetic, anthranilic, propanoic, 2-hydroxypropanoic, 2-oxopropanoic, propandioic, cyclopentanepropionic, cyclopentane propionic, 3-phenylpropionic, butanoic, butandioic, benzoic, 3-(4-hydroxybenzoyl)benzoic, 2-acetoxy-benzoic, ascorbic, cinnamic, lauryl sulfuric, stearic, muconic, mandelic, succinic, embonic, fumaric, malic, maleic, hydroxymaleic, malonic, lactic, citric, tartaric, glycolic, glyconic, gluconic, pyruvic, glyoxalic, oxalic, mesylic, succinic, salicylic, phthalic, palmoic, palmeic, thiocyanic, methanesulphonic, ethanesulphonic, 1,2-ethanedisulfonic, 2-hydroxyethanesulfonic, benzenesulphonic, 4-chorobenzenesulfonic, napthalene-2-sulphonic, p-toluenesulphonic, camphorsulphonic, 4-methylbicyclo[2.2.2]-oct-2-ene-1-carboxylic, glucoheptonic, 4,4′-methylenebis-3-(hydroxy-2-ene-1-carboxylic acid), hydroxynapthoic.

“Pharmaceutically-acceptable bases” include inorganic and organic bases which are non-toxic at the concentration and manner in which they are formulated. For example, suitable bases include those formed from inorganic base forming metals such as lithium, sodium, potassium, magnesium, calcium, ammonium, iron, zinc, copper, manganese, aluminum, N-methylglucamine, morpholine, piperidine and organic non-toxic bases including, primary, secondary and tertiary amine, substituted amines, cyclic amines and basic ion exchange resins, [e.g., N(R′)₄+(where W is independently H or C14 alkyl, e.g., ammonium, Tris)], for example, isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic non-toxic bases are isopropylamine, diethylamine, ethanolamine, trimethamine, dicyclohexylamine, choline, and caffeine.

Additional pharmaceutically acceptable acids and bases useable with the present invention include those which are derived from the amino acids, for example, histidine, glycine, phenylalanine, aspartic acid, glutamic acid, lysine and asparagine.

Formulations herein may also include one or more buffers or salts. Buffers and salts include those derived from both acid and base addition salts of the above indicated acids and bases. Specific buffers and/or salts include arginine, histidine, succinate and acetate.

If a formulation is to be lyophilized, a lyoprotectant may be added. A “lyoprotectant” is a molecule which, when combined with a protein of interest, significantly prevents or reduces physicochemical instability of the protein upon lyophilization and subsequent storage. Exemplary lyoprotectants include sugars and their corresponding sugar alcohols; an amino acid such as monosodium glutamate or histidine; a methylamine such as betaine; a lyotropic salt such as magnesium sulfate; a polyol such as trihydric or higher molecular weight sugar alcohols, e.g., glycerin, dextran, erythritol, glycerol, arabitol, xylitol, sorbitol, and mannitol; propylene glycol; polyethylene glycol; Pluronics®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred lyoprotectant are the non-reducing sugars trehalose or sucrose.

A “pharmaceutically acceptable sugar” is a molecule which, when combined with a protein of interest, significantly prevents or reduces physicochemical instability of the protein upon storage. When the formulation is intended to be lyophilized and then reconstituted. “pharmaceutically acceptable sugars” may also be known as a “lyoprotectant”. Exemplary sugars and their corresponding sugar alcohols includes: an amino acid such as monosodium glutamate or histidine; a methylamine such as betaine; a lyotropic salt such as magnesium sulfate; a polyol such as trihydric or higher molecular weight sugar alcohols, e.g., glycerin, dextran, erythritol, glycerol, arabitol, xylitol, sorbitol, and mannitol; propylene glycol; polyethylene glycol; Pluronics®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred pharmaceutically-acceptable sugars are the non-reducing sugars trehalose or sucrose.

A “preservative” is a compound which can be added to the formulations herein to reduce bacterial activity. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methy 1 or propyl paraben, catechol, resorcinol, cyclohexanol, 3-pentanol, and m-cresol.

Additional proteins such as albumin (human serum albumin or bovine serum albumin, for example) or an immunoglobulin (an IgG constant region, for example) may be added to further stabilize the protein of interest.

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Tonicity agents may also be included, for example, to adjust or maintain the tonicity of a liquid composition. When used with large, charged biomolecules such as proteins and antibodies, such agents may interact with the charged groups of the amino acid side chains, thereby lessening the potential for inter and infra-molecular interactions. Tonicity agents can be present in any amount between 0.1% to 25% by weight, preferably 1 to 5%, taking into account the relative amounts of the other ingredients. Exemplary tonicity agents include polyhydric sugar alcohols, preferably trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thiosulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose: and polysaccharides such as dextrin or dextran.

The formulation herein may also contain more than one active compound as necessary for the particular indication being treated, preferably those with complementary activities that do not adversely affect each other. Thus, it may comprise more than one antibody or more than one protein, for example.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 18th edition, supra. Liposomal or proteinoid compositions may also be used to formulate the proteins or antibodies disclosed herein. See U.S. Pat. Nos. 4,925,673 and 5,013,556.

Stability of the proteins and antibodies described herein may be enhanced through the use of non-toxic “water-soluble poly valent metal salts”. Examples include Ca²⁺, Mg²⁺, Zn²⁺, Fe³⁺, Fe²⁺, Cu²⁺, Sn²⁺, Sn³⁺, Al²⁺ and Al³⁺. Example anions that can form water soluble salts with the above polyvalent metal cations include those formed from inorganic acids and/or organic acids. Such water-soluble salts have a solubility in water (at 20° C.) of at least about 20 mg/ml, alternatively at least about 100 mg/ml, alternative at least about 200 mg/ml. Suitable inorganic acids that can be used to form the “water soluble polyvalent metal salts” include hydrochloric, acetic, sulfuric, nitric, thiocyanic and phosphoric acid. Suitable organic acids that can be used include aliphatic carboxylic acid and aromatic acids. Aliphatic acids within this definition may be defined as saturated or unsaturated C₂₋₉ carboxylic acids (e.g., aliphatic mono-, di- and tri-carboxylic acids). For example, exemplary monocarboxylic acids within this definition include the saturated C₂₋₉ monocarboxylic acids acetic, propionic, butyric, valeric, caproic, enanthic, caprylic pelargonic and capryonic, and the unsaturated C₂₋₉ monocarboxylic acids acrylic, propriolic methacrylic, crotonic and isocrotonic acids. Exemplary dicarboxylic acids include the saturated C₂₋₉ dicarboxylic acids malonic, succinic, glutaric, adipic and pimelic, while unsaturated C₂₋₉ dicarboxylic acids include maleic, fumaric, citraconic and mesaconic acids. Exemplary tricarboxylic acids include the saturated C24 tricarboxylic acids tricarballylic and 1,2,3-butanetricarboxylic acid. Additionally, the carboxylic acids of this definition may also contain one or two hydroxyl groups to form hydroxy carboxylic acids. Exemplary hydroxy carboxylic acids include glycolic, lactic, glyceric, tartronic, malic, tartaric and citric acid. Aromatic acids within this definition include benzoic and salicylic acid.

### Therapeutic Methods and Routes of Administration

Any of the antibodies and antibody formulations provided herein may be used in therapeutic methods, with the type of therapy depending, for example, in part on the antigen binding properties or antigen target of the antibody. Antibodies, generally, may be used in a wide variety of therapeutic indications, such as treatments for autoimmune conditions, neurological disorders and neurodegenerative diseases, cancers, and infectious diseases, among others.

Antibodies herein can be administered alone or used in a combination therapy. For instance, the combination therapy includes administering an antibody and administering at least one additional therapeutic agent (e.g. one, two, three, four, five, or six additional therapeutic agents). Such combination therapies noted above encompass combined administration (where two or more therapeutic agents are included in the same or separate pharmaceutical compositions), and separate administration, in which case, administration of the antibody of the invention can occur prior to, simultaneously, and/or following, administration of the additional therapeutic agent or agents.

Antibodies can be formulated to be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic.

### Kits and Articles of Manufacture

In another aspect of the disclosure, recombinant antibodies herein may be used in vitro, i.e. in the laboratory to modify the behavior of cells, or for use in diagnostics, for example. For instance, there are numerous instances in which it may be beneficial to assay the behavior of a cell or tissue sample in which the level of a particular antigen is useful to determine. Thus, the present disclosure also encompasses kits comprising one or more recombinant antibodies of the disclosure. Kits may comprise the antibody, optionally also with instructions for use, appropriate buffers, and/or labeling molecules.

### EXAMPLES

### Example 1

### LPLA2 and PLBL2 Complexing of Multiple Antibodies

To establish the impact of antibody disulfide bond structure on binding, a panel of IgG1, IgG2, and IgG4 antibodies were evaluated using SPR to generate equilibrium dissociation constants (K_{D}) and assess relative affinity. Antibodies of class IgG4 bound most strongly to PLBL2-01 (PLBL2 lot 1) compared to IgG1 and IgG2 antibodies (Table 1). When SPR analysis was attempted on LPLA2-01 (LPLA2 lot 1), no binding was observed. MST was used as an alternative to SPR, and was able to detect binding for LPLA2-01 and thioesterase. While the IgG4 antibodies remained the tightest PLBL2-01 binders by MST, the disparities between the classes were minimized by MST detection (Table 2). For LPLA2-01, MAb2 (IgG1), MAb5 (IgG1), and MAb1 (IgG4) were the tightest binders, with <5 μM K_{d} values. Thioesterase only bound tightly to MAb2 and MAb5, with <10 μM K_{d} values.

TABLE 1
Biacore binding of mAbs to PLBL2-01.

| mAb | Subclass | K_{D} (uM); SPR | K_{D} (uM); MST |
|---|---|---|---|
| mAb1 | IgG4 | 1.2 ± 0.4 | 1.3 ± 0.3 |
| mAb2 | IgG1 | 67 | 1.5 |
| mAb3 | IgG1 | 82 ± 11 | 22 ± 9 |
| mAb4 | IgG1 | 55 ± 25 | >5 uM |
| mAb5 | IgG1 | 73 | N.D. |
| mAb6 | IgG2 | 91 ± 27 | 6 ± 2 |
| mAb7 | IgG4 | 2.3 ± 0.2 | 0.76 |

TABLE 2
MST binding of mAbs to lipases.

| mAb | Isotype | PLBL2 | LPLA2 | Thioesterase |
|---|---|---|---|---|
| mAb1 | IgG4 | 1.3 ± 0.3 | 1.7 ± 0.7 | >100 |
| mAb2 | IgG1 | 1.5 | 4.4 ± 1 | 1.3 ± 2.5 |
| mAb3 | IgG1 | 22 ± 9 | 29 ± 11.3 | 72.8 ± 3.8 |
| mAb4 | IgG1 | >5.5 | — | — |
| mAb5 | IgG1 | — | 2.3 | 7.6 |
| mAb6 | IgG2 | 6 ± 2 | 20 | — |
| mAb7 | IgG4 | 0.8 | 20.3 ± 4.7 | 46.9 ± 1.8 |

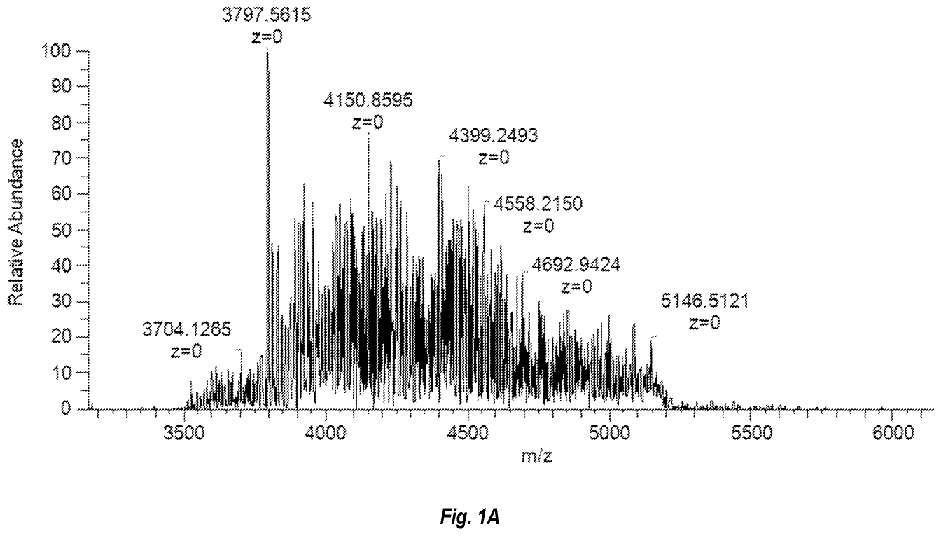

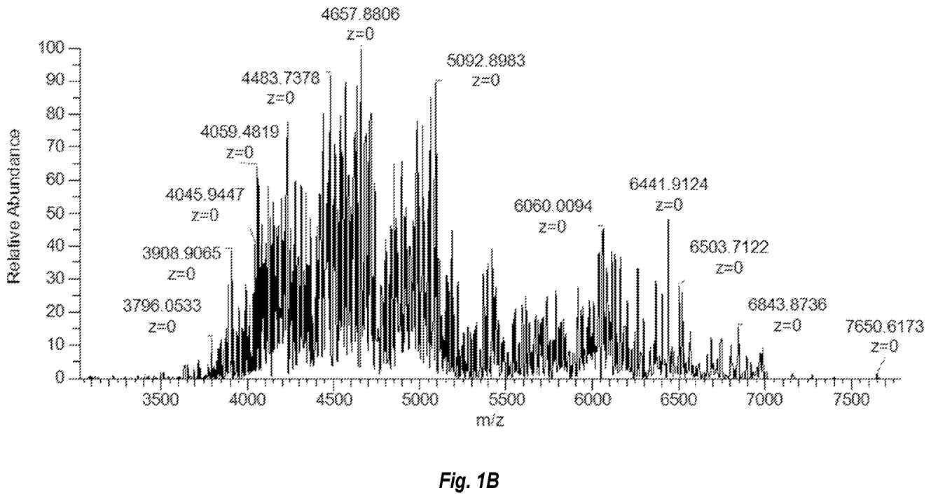

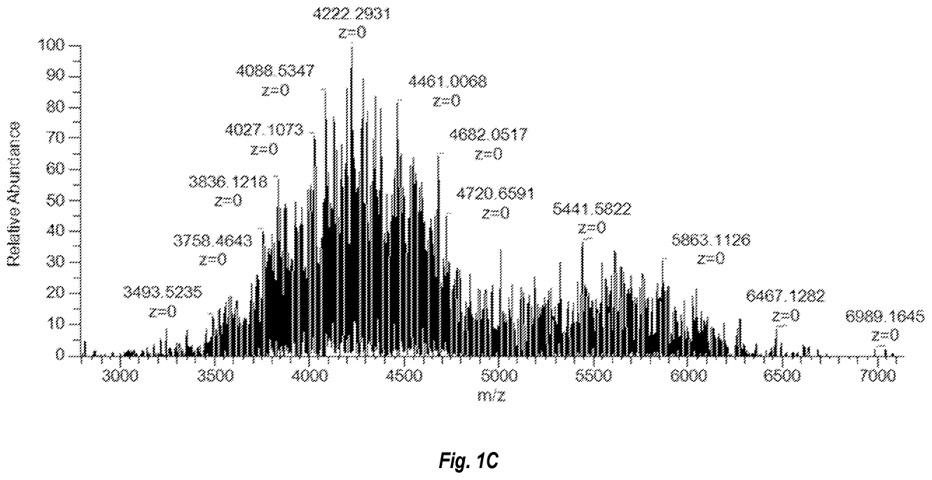

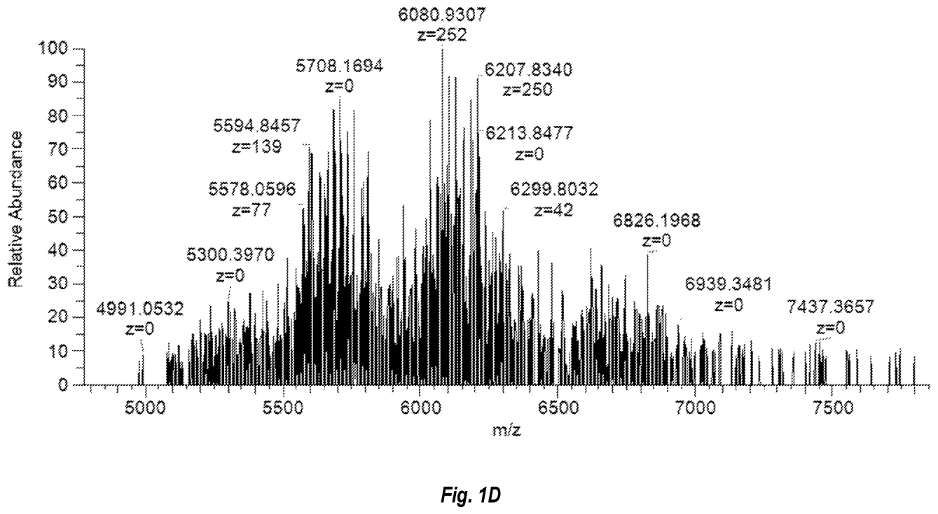

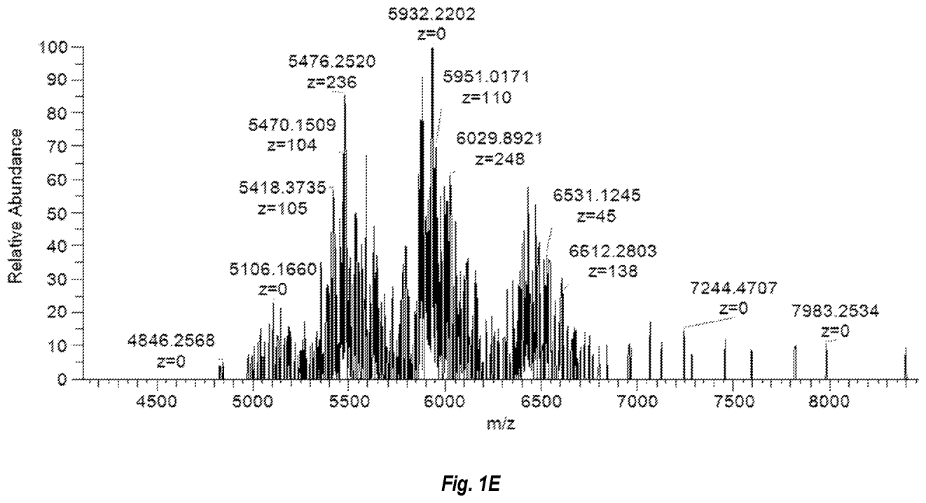

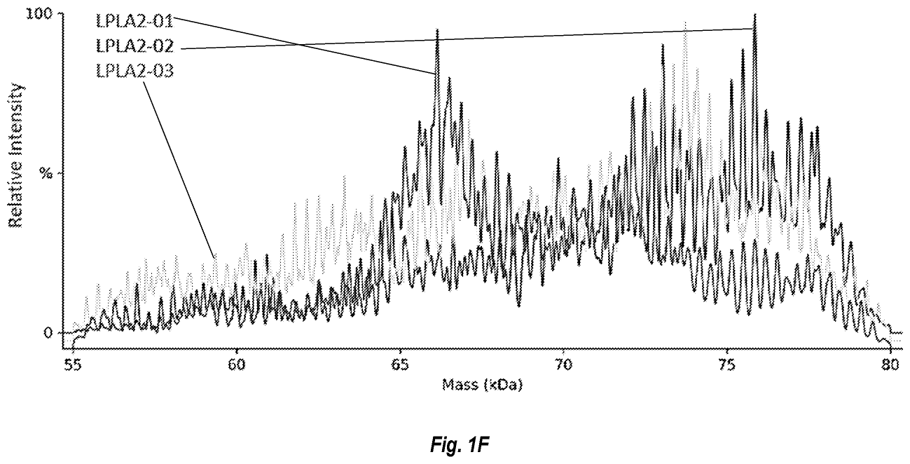

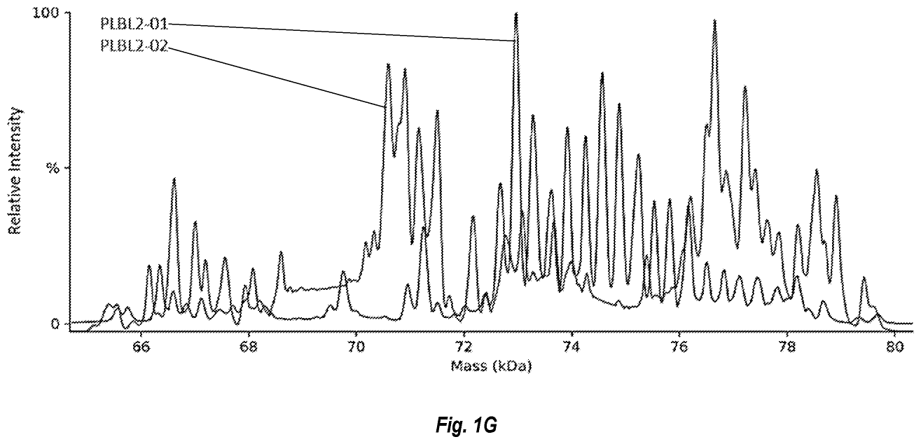

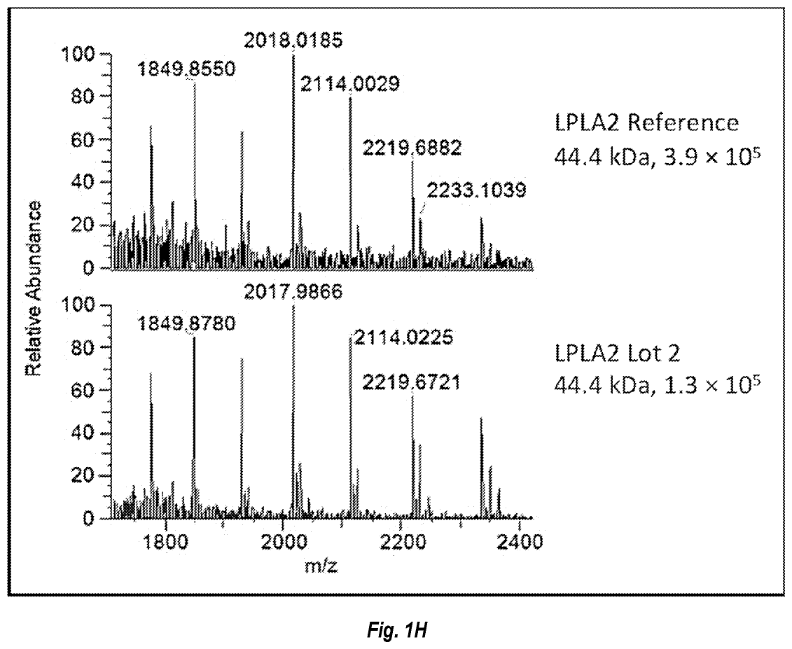

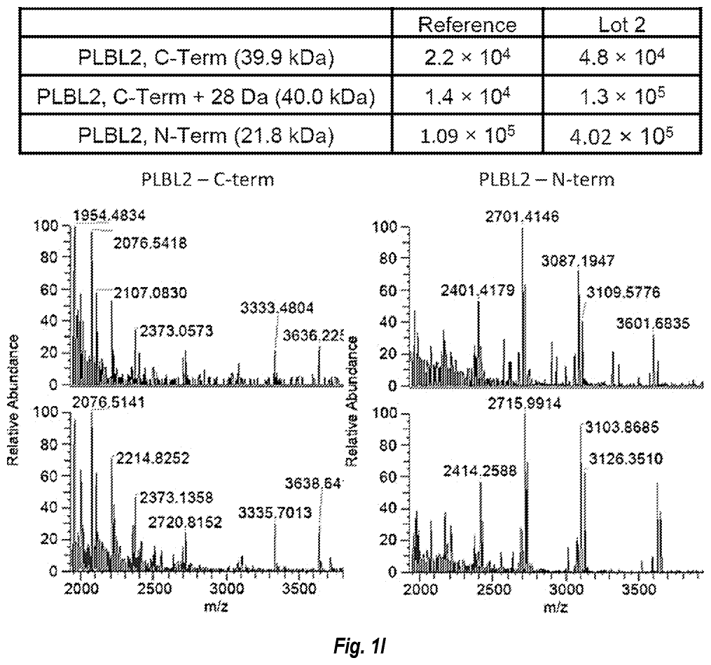

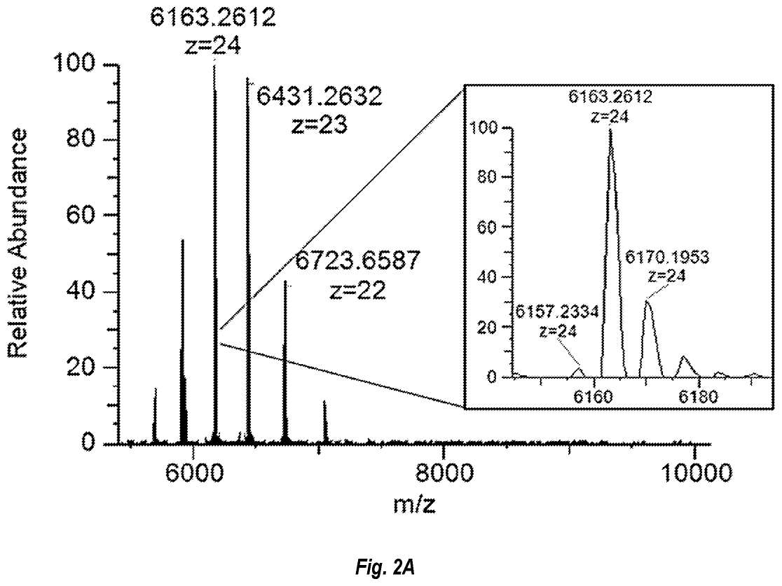

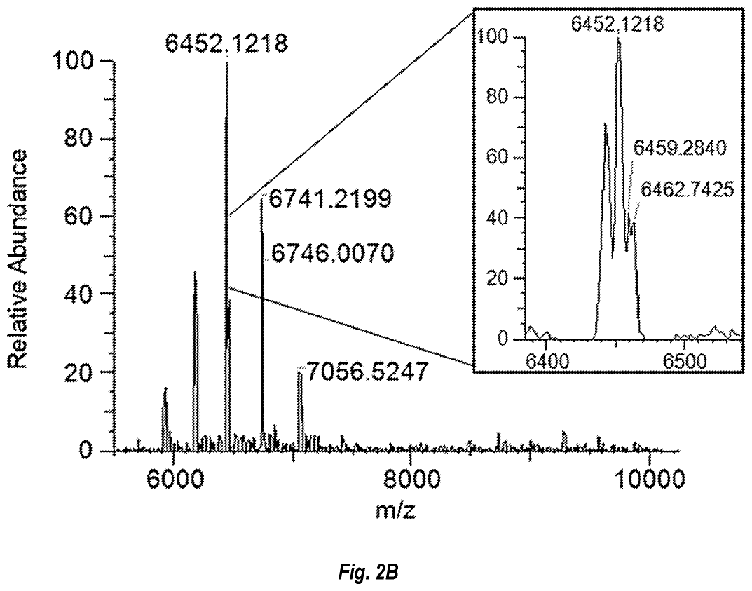

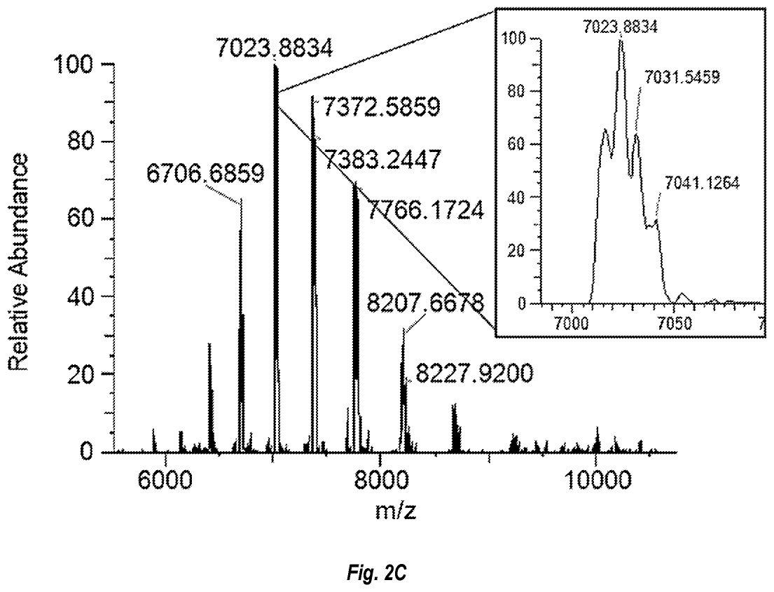

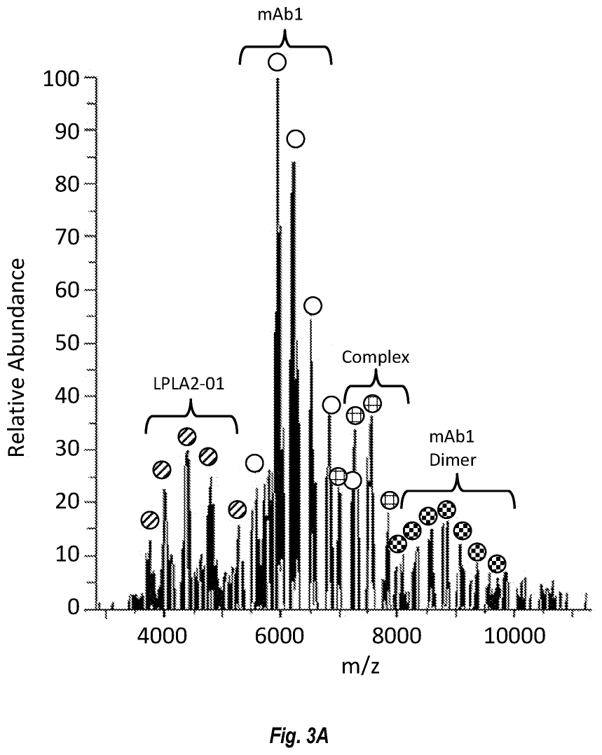

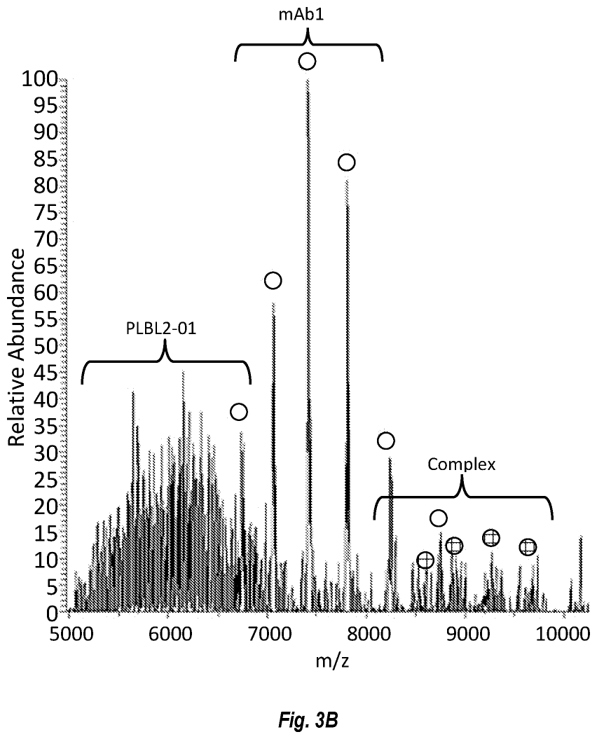

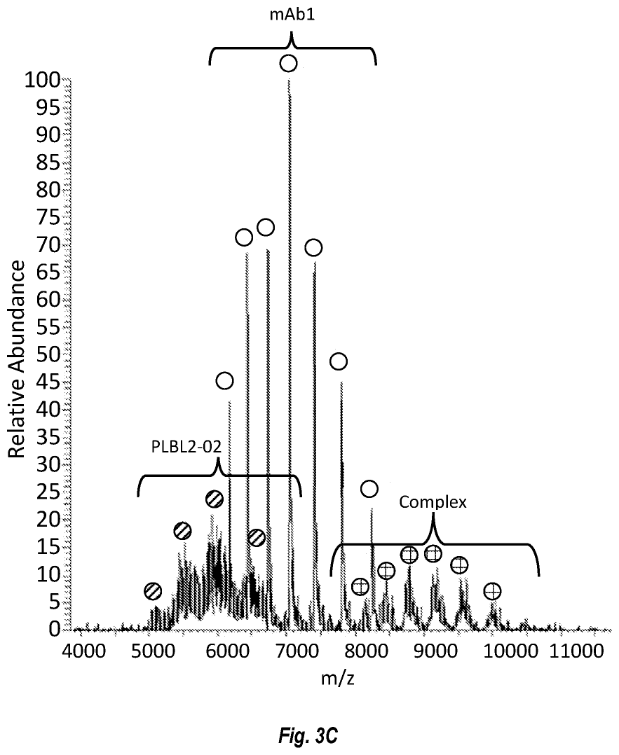

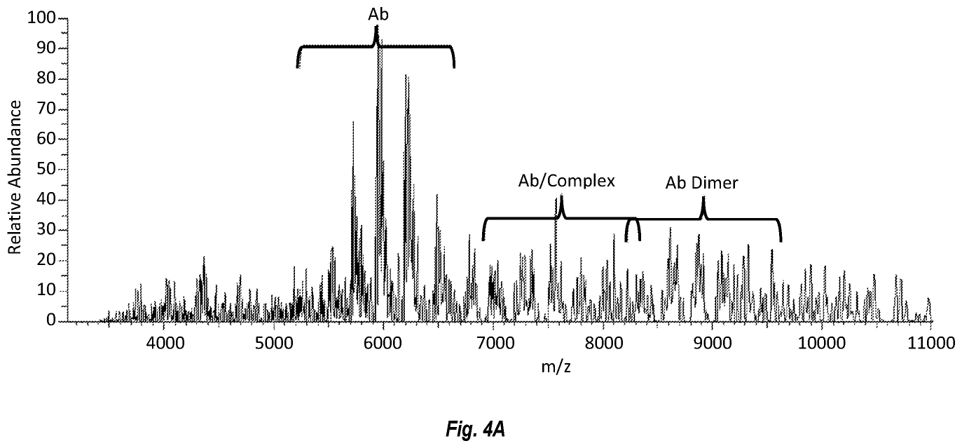

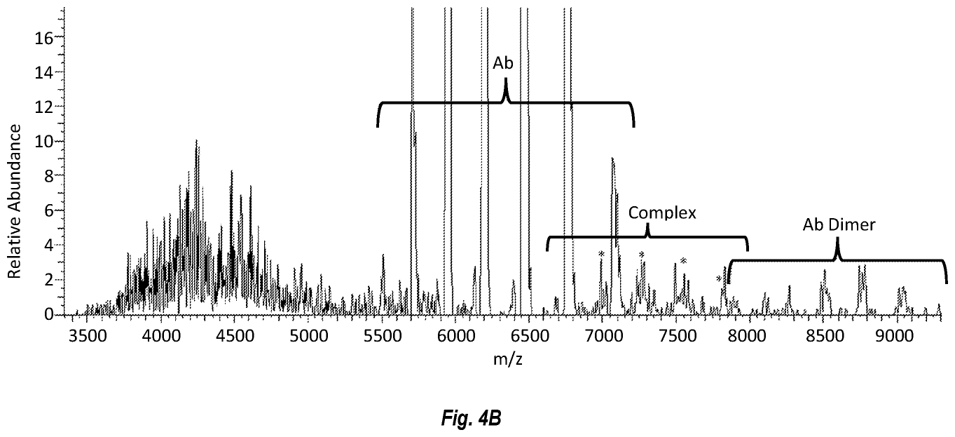

Native mass spectrometry was selected as an orthogonal strategy to validate complex formation because it offered an opportunity to detect non-immobilized or unlabeled complexes and to determine the stoichiometry. Initial characterization by native MS was used to first characterize the lipases (FIG. 1) and the antibodies independently (FIG. 2). The lipases were observed to have extensive glycosylation patterns, leading to heterogeneous spectra. LPLA2 and PLBL2 had a deconvolution mass range from 62-76 kDa and 66-80 kDa, respectively. Detection of the complex was achieved by optimizing MS transmission parameters (FIG. 3). Complex formation was tested at a 1:10 and 10:1 lipase:antibody molar ratio (FIG. 4). Even at the 10:1 lipase:Ab ratio, the antibody charge state distribution appeared with higher intensity compared to the lipase because of its preferred MS ionization and the split lipase signal resulting from the glycoforms. As such, all work was performed at a 10:1 ratio. Complexes of LPLA2-01 and PLBL2 to antibody showed high heterogeneity (FIG. 3), indicating that multiple lipase glycoforms complexed to the antibody. A 1:1 stoichiometry was observed with a complex average mass of approximately 218 kDa for PLBL2 and 213 kDa for LPLA2 antibody complexes.

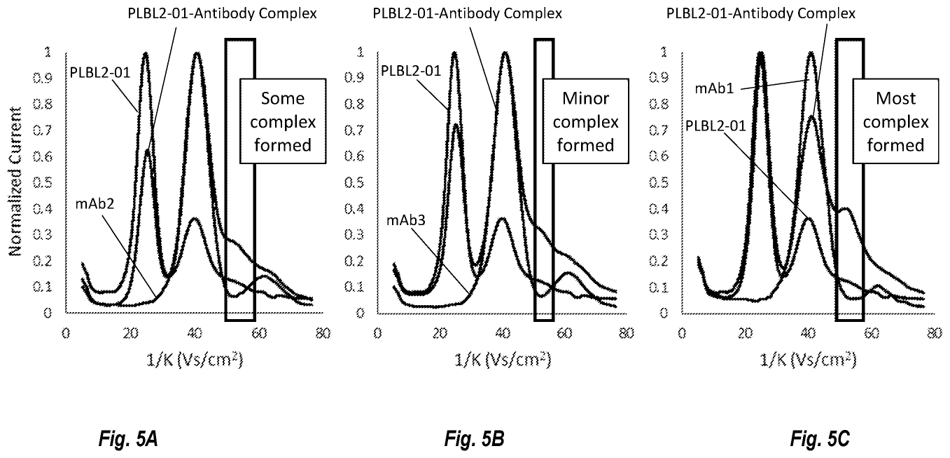

The heterogeneity of the lipase created challenges in the deconvolution of mass spectral data. Furthermore, the complex was only preserved through static spray MS, versus Triversa NanoMate™ (Advion, Inc., Ithaca, N.Y.) or LC infusion experiments, which limited the throughput of this method. Therefore a non-MS electrospray ion mobility spectroscopy instrument was evaluated as a first-in-kind screening technology for noncovalent protein complexes. In IM, electrosprayed proteins are produced with a single charge and follow a trajectory around a central rod, in a given electric field, based on their collisional cross sectional area (22). The inverse mobility (1/K) of ions across a swept voltage range was modeled, where higher inverse mobilities generally correlated with larger species. For each experiment, the control lipase, antibody, and complex species were normalized and compared (FIG. 5). PLBL2-01 (25.3 l/K) binding to MAb1(IgG4), MAb2 (IgG1), and MAb3 (IgG1) gave rise to a peak at 51.4 l/K, between the monomer Ab (40.9 I/K) and the electrospray gas-phase dimer (60.2 l/K). A minor peak was observed for the PLBL2-01-MAb3 complex, suggesting that 1M may have a lower limit of detection (LOD) than MST. The amount of complex agreed with the prior results, where PLBL2-01 complex showed a clear rank order in formation of MAb1>MAb2>MAb3.

### LPLA2 Structure Effects on Complex Binding

Atmospheric ion mobility analysis offered an opportunity to directly assess the binding of different lipase conformers to antibodies. A comparison of the mean inverse mobility of the monomer PLBL2-01 peak was made pre and post complexation. If all gycoforms of lipase bound equally to antibody, the peak would be expected to have a reduced amplitude, but maintain the same width and mean 1/K value. The rightward shift observed for the lipase peak (FIG. 5) indicated that smaller-sized, or lesser-glycosylated, lipases complexed preferentially with mAbs compared to their larger counterparts. To validate this observation, the effects of lipase glycosylation on complexation were further explored by more traditional analyses, including intact mass, glycan composition and exoglycosidase-treatment.

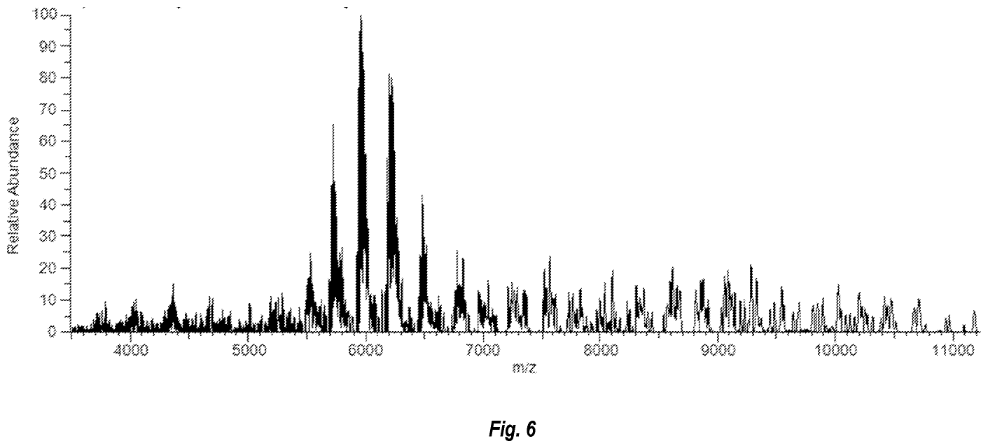

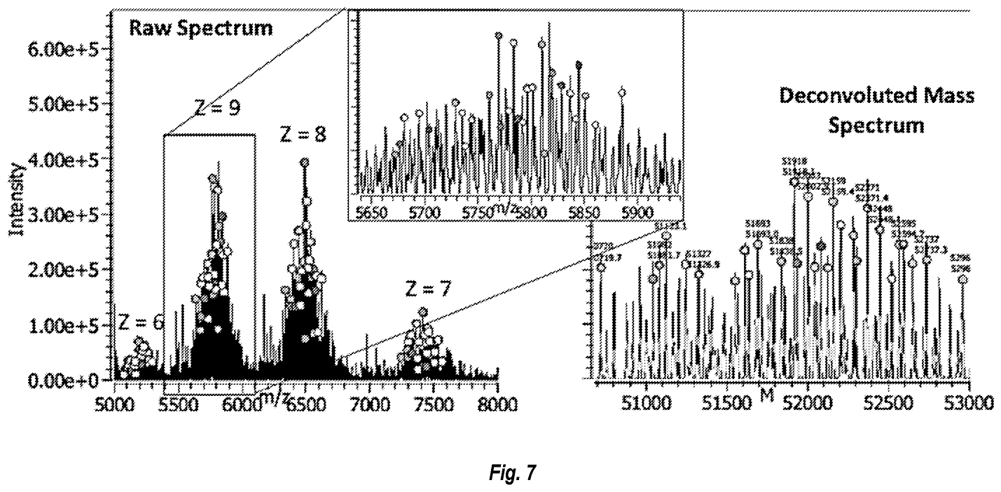

Native mass spectrometry was used to assess if a complex could be formed after exoglycosidase treatment. Deglycosylation of the lipases LPLA2-01 and PLBL2-01 by PNGaseF prevented binding to all antibodies tested. Interestingly, desilylation of the lipases by neuraminidase had no effect on the binding (FIG. 6). Neuraminidase-treated lipase also showed a less-heterogeneous spectra that was suitable for deconvolution, where 22 glycoforms were assigned (FIG. 7).

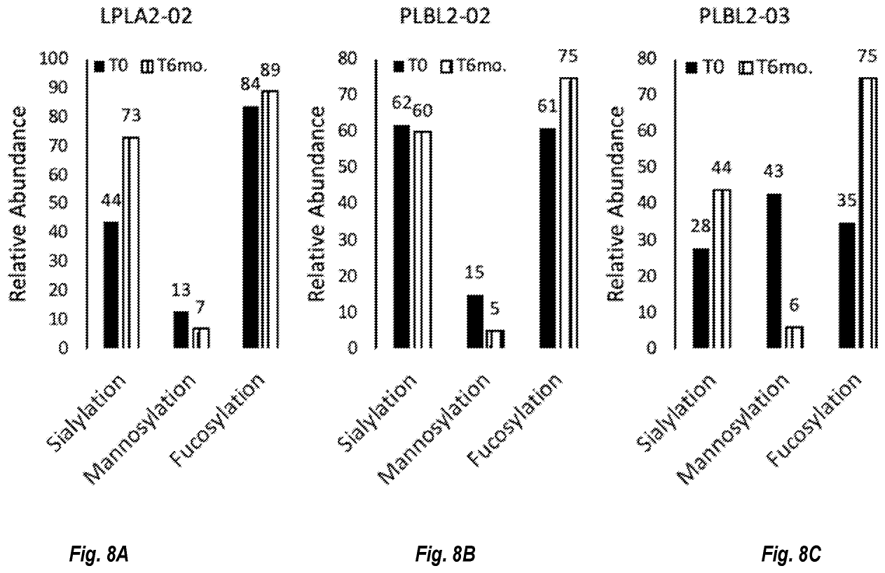

Glycosylation of proteins is known to vary across different lots of production. A second lot of lipase samples were purified in order to perform free glycan composition analysis. As shown in FIG. 1F, LPLA2-01 and -03 had significantly different glycoforms when compared to LPLA2-02, especially in the lower mass region. Overall, LPLA2-02 had significantly less glycoforms of mass 65-70 kDa and was enriched in high-mass forms at 74-80 kDa. (FIG. 1F). PLBL2-02 had a more even distribution of glycoforms spread over the mass range versus PLBL2-01, which showed a concentration of species between 72-77 kDa. (FIG. 1G.) Interestingly, no binding of LPLA2-02 to antibodies could be observed by MST or native MS, whereas PLBL2-02 bound tighter when compared to PLBL2-01. Differences in the relative proportion of mannose, sialylated, and fucosylated species were detected between the samples (FIG. 8, Table 3). PLBL2-02 had the highest proportion of mannosylated species detected, at 43%. LPLA2-02 had a higher total ratio of fucose-to-sialic acid containing glycans versus PLBL2-01, at 2:1 versus 1:1 levels, although both had similarly low mannosylation, at 13 and 15% of their total compositions respectively. Interestingly, no binding of LPLA2-02 to the mAbs could be observed by MST or native MS, whereas PLBL2-02 bound tighter.

TABLE 3
Glycan composition of PLBL2-01, PLBL2-02, and LPLA2-01 at time point zero and six months (inactive).

| | | | | | Sialylation | | Mannosylation | | Fucosylation | |
|---|---|---|---|---|---|---|---|---|---|---|
| Glycan Name | Rel. % T0 | Rel. % T6 mo. | Mass | Composition | T0 | T6 mo. | T0 | T6 mo. | T0 | T6 mo. |

LPLA2-01

| | | | | | | | | | | |
|---|---|---|---|---|---|---|---|---|---|---|
| Man3 | 2.91 | 0.00 | 910.3 | Hex3HexNAc2 | | | 2.91 | 0.00 | | |
| Man4 | 1.12 | 0.33 | 1072.4 | Hex4HexNAc2 | | | 1.12 | 0.33 | | |
| Man5 | 4.37 | 0.00 | 1234.4 | Hex5HexNAc2 | | | 4.37 | 0.00 | | |
| 2000 0A 0G | 0.82 | 0.59 | 1316.5 | Hex3HexNAc4G0 | | | | | | |
| 2100 0A 0G | 9.97 | 0.00 | 1462.5 | Hex3HexNAc4dHex1G0F | | | | | 9.97 | 0.00 |
| 2010 0A 0G | 0.00 | 1.62 | 1478.5 | Hex4HexNAc4G1 | | 0.00 | 0.00 | 0.00 | | 0.00 |
| Man7 | 0.87 | 0.00 | 1558.5 | Hex7HexNAc2 | | | 0.87 | 0.00 | | |
| 2110 0A 0G | 1.82 | 1.30 | 1624.6 | Hex4HexNAc4dHex1G1F | | | | | 1.82 | 1.30 |
| 3100 0A 0G | 16.31 | 0.00 | 1665.6 | Hex3HexNAc5dHex1 | | | | | 16.31 | 0.00 |
| 2120 0A 0G | 0.00 | 0.94 | 1786.7 | Hex5HexNAc4dHex1G2F | | | 0.00 | 0.00 | 0.00 | 0.94 |
| 3110 0A 0G | 6.96 | 11.54 | 1827.7 | Hex4HexNAc5dHex1 | | | | | 6.96 | 11.54 |
| Man9 | 3.40 | 5.48 | 1882.6 | Hex9HexNAc2 | | | 3.40 | 5.48 | | |
| 2111 1A 0G | 0.99 | 1.75 | 1915.7 | Hex4HexNAc4dHex1NeuAc1 | 0.99 | 1.75 | | | 0.99 | 1.75 |
| 2021 1A 0G/ | 0.00 | 1.36 | 1931.7 | Hex5HexNAc4NeuAc1/ | 0.00 | 1.36 | | | 0.00 | 1.36 |
| 2111 0A 1G | | | | Hex4HexNAc4dHex1NeuGc1 | | | | | | |
| 3011 1A 0G | 2.79 | 1.78 | 1972.7 | Hex4HexNAc5NeuAc1 | 2.79 | 1.78 | | | | |
| 3120 0A 0G | 5.13 | 0.58 | 1989.7 | Hex5HexNAc5dHex1 | | | | | 5.13 | 0.58 |
| Man9Glc1 | 0.00 | 1.56 | 2044.7 | Hex10HexNAc2 | 0.00 | | 0.00 | 1.56 | 0.00 | |
| 2121 1A 0G | 14.26 | 0.00 | 2077.7 | Hex5HexNAc4dHex1NeuAc1 | 14.26 | 0.00 | | | 14.26 | 0.00 |
| 3111 1A 0G | 0.00 | 19.91 | 2118.8 | Hex4HexNAc5dHex1NeuAc1 | 0.00 | 19.91 | 0.00 | | 0.00 | 19.91 |
| 3021 1A 0G/ | 0.00 | 0.33 | 2134.8 | Hex5HexNAc5NeuAc1/ | 0.00 | 0.33 | 0.00 | | 0.00 | 0.33 |
| 3111 0A 1G | | | | Hex4HexNAc5dHex1NeuGc1 | | | | | | |
| 3130 0A 0G | 2.69 | 2.89 | 2151.8 | Hex6HexNAc5dHex1 | | | | | 2.69 | 2.89 |
| 3121 1A 0G | 6.22 | 5.39 | 2280.8 | Hex5HexNAc5dHex1NeuAc1 | 6.22 | 5.39 | | | 6.22 | 5.39 |
| 3031 1A 0G/ | 0.00 | 0.78 | 2296.8 | Hex6HexNAc5NeuAc1/ | 0.00 | 0.78 | 0.00 | | 0.00 | 0.78 |
| 3121 0A 1G | | | | Hex5HexNAc5dHex1NeuGc1 | | | | | | |
| 3131 1A 0G | 19.36 | 41.88 | 2442.9 | Hex6HexNAc5dHex1NeuAc1 | 19.36 | 41.88 | | | 19.36 | 41.88 |

PLBL2-01

| | | | | | | | | | | |
|---|---|---|---|---|---|---|---|---|---|---|
| Man3 | 1.59 | 0.00 | 910.3 | Hex3HexNAc2 | | | 1.59 | 0.00 | | |
| Man4 | 3.45 | 2.12 | 1072.4 | Hex4HexNAc2 | | | 3.45 | 2.12 | | |
| Man5 | 3.41 | 0.00 | 1234.4 | Hex5HexNAc2 | | | 3.41 | 0.00 | | |
| 1100 0A 0G | 0.00 | 1.62 | 1259.5 | Hex3HexNAc3dHex1 | | 0.00 | | 0.00 | 0.00 | 1.62 |
| 1010 0A 0G | 0.00 | 3.29 | 1275.5 | Hex4HexNAc3 | | 0.00 | | 0.00 | | |
| | | | | nonspecific hexose | | | | | | |
| | | | | is Man or Gal | | | | | | |
| 2000 0A 0G | 1.47 | 1.15 | 1316.5 | Hex3HexNAc4G0 | | | | | | |
| Man6 | 3.91 | 2.51 | 1396.5 | Hex6HexNAc2 | | | 3.91 | 2.51 | | |
| 2100 0A 0G | 1.28 | 0.00 | 1462.5 | Hex3HexNAc4dHex1G0F | | | | | 1.28 | 0.00 |
| 2010 0A 0G | 2.17 | 0.00 | 1478.5 | Hex4HexNAc4G1 | | | | | | |
| Man7 | 1.82 | 0.00 | 1558.5 | Hex7HexNAc2 | | | 1.82 | 0.00 | | |
| 1011 1A 0G | 0.90 | 2.71 | 1566.6 | Hex4HexNAc3NeuAc1 | 0.90 | 2.71 | | | | |
| 2110 0A 0G | 2.21 | 2.32 | 1624.6 | Hex4HexNAc4dHex1G1F | | | | | 2.21 | 2.32 |
| 2020 0A 0G | 13.24 | 13.09 | 1640.6 | Hex5HexNAc4G2 | | | | | | |
| Man8 | 0.62 | 0.00 | 1720.6 | Hex8HexNAc2 | | | 0.62 | 0.00 | | |
| 1111 0A 1G | 2.06 | 0.00 | 1728.6 | Hex4HexNAc3dHex1NeuGc1 | 2.06 | 0.00 | | | 2.06 | 0.00 |
| 2011 1A 0G | 0.78 | 0.00 | 1769.6 | Hex4HexNAc4NeuAc1 | 0.78 | 0.00 | | | | |
| 2120 0A 0G | 3.24 | 11.25 | 1786.7 | Hex5HexNAc4dHex1G2F | | | | | 3.24 | 11.25 |
| 2111 1A 0G | 1.22 | 2.84 | 1915.7 | Hex4HexNAc4dHex1NeuAc1 | 1.22 | 2.84 | | | 1.22 | 2.84 |
| 2021 1A 0G/ | 23.36 | 48.70 | 1931.7 | Hex5HexNAc4NeuAc1/ | 23.36 | 48.70 | | | 23.36 | 48.70 |
| 2111 0A 1G | | | | Hex4HexNAc4dHex1NeuGc1 | | | | | | |
| 2021 0A 1G | 0.93 | 0.00 | 1947.7 | Hex5HexNAc4NeuGc1 | 0.93 | 0.00 | | | | |
| 2130 0A 0G | 0.00 | 1.48 | 1948.7 | Hex6HexNAc4dHex1 | | 0.00 | | 0.00 | 0.00 | 1.48 |
| 2121 1A 0G | 22.65 | 0.00 | 2077.7 | Hex5HexNAc4dHex1NeuAc1 | 22.65 | 0.00 | | | 22.65 | 0.00 |
| 3130 0A 0G | 0.00 | 0.81 | 2151.8 | Hex6HexNAc5dHex1 | | 0.00 | | 0.00 | 0.00 | 0.81 |
| 2022 2A 0G | 4.50 | 0.00 | 2222.8 | Hex5HexNAc4NeuAc2 | 4.50 | 0.00 | | | | |
| 2122 2A 0G | 3.87 | 0.00 | 2367.9 | Hex5HexNAc4dHex1NeuAc2 | 3.87 | 0.00 | | | 3.87 | 0.00 |
| 3131 1A 0G | 1.32 | 6.10 | 2442.9 | Hex6HexNAc5dHex1NeuAc1 | 1.32 | 6.10 | | | 1.32 | 6.10 |

PLBL2-02

| | | | | | | | | | | |
|---|---|---|---|---|---|---|---|---|---|---|
| Man3 | 8.59 | 0.00 | 910.3 | Hex3HexNAc2 | | | 8.59 | 0.00 | | |
| 0100 0A 0G | 0.00 | 0.31 | 1056.4 | Hex3HexNAc2dHex1 | | | | 0.00 | 0.00 | 0.31 |
| Man4 | 15.14 | 0.00 | 1072.4 | Hex4HexNAc2 | | | 15.14 | | | |
| 1000 0A 0G | 0.00 | 0.34 | 1113.4 | Hex3HexNAc3 | 0.00 | | | 0.00 | 0.00 | |
| Man5 | 16.26 | 0.00 | 1234.4 | Hex5HexNAc2 | | | 16.26 | 0.00 | | |
| 1100 0A 0G | 0.00 | 4.51 | 1259.5 | Hex3HexNAc3dHex1 | 0.00 | | | 0.00 | 0.00 | 4.51 |
| 1010 0A 0G | 0.95 | 0.00 | 1275.5 | Hex4HexNAc3 | | | | | | |
| | | | | nonspecific hexose | | | | | | |
| | | | | is Man or Gal | | | | | | |
| 2000 0A 0G | 3.33 | 0.00 | 1316.5 | Hex3HexNAc4G0 | | | | | | |
| Man6 | 2.20 | 0.00 | 1396.5 | Hex6HexNAc2 | | | 2.20 | | | |
| 2100 0A 0G | 0.90 | 0.00 | 1462.5 | Hex3HexNAc4dHex1G0F | | | | | 0.90 | 0.00 |
| 2010 0A 0G | 1.58 | 0.00 | 1478.5 | Hex4HexNAc4G1 | | | | | | |
| 3000 0A 0G | 0.00 | 0.25 | 1519.6 | Hex3HexNAc5 | 0.00 | | | 0.00 | 0.00 | |
| 1011 1A 0G | 0.00 | 3.10 | 1566.6 | Hex4HexNAc3NeuAc1 | 0.00 | 3.1.0 | | 0.00 | 0.00 | |
| 2110 0A 0G | 3.68 | 0.00 | 1624.6 | Hex4HexNAc4dHex1G1F | | | | | 3.68 | |
| 2020 0A 0G | 14.36 | 0.00 | 1640.6 | Hex5HexNAc4G2 | | | | | | |
| 3010 0A 0G | 0.00 | 0.66 | 1681.6 | Hex4HexNAc5 | 0.00 | | | 0.00 | 0.00 | |
| Man8 | 0.83 | 0.00 | 1720.6 | Hex8HexNAc2 | | | 0.83 | 0.00 | | |
| 1111 0A 1G | 1.15 | 0.00 | 1728.6 | Hex4HexNAc3dHex1NeuGc1 | 1.15 | 0.00 | | | 1.15 | 0.00 |
| 2120 0A 0G | 3.80 | 0.00 | 1786.7 | Hex5HexNAc4dHex1G2F | | | | | 3.80 | |
| 3110 0A 0G | 0.00 | 0.85 | 1827.7 | Hex4HexNAc5dHex1 | 0.00 | | | 0.00 | 0.00 | 0.85 |
| 2111 1A 0G | 1.35 | 0.00 | 1915.7 | Hex4HexNAc4dHex1NeuAc1 | 1.35 | | | | 1.35 | |
| 2021 1A 0G/ | 16.31 | 0.00 | 1931.7 | Hex5HexNAc4NeuAc1/ | 16.31 | | | | 16.31 | |
| 2111 0A 1G | | | | Hex4HexNAc4dHex1NeuGc1 | | | | | | |
| 2130 0A 0G | 0.00 | 0.28 | 1948.7 | Hex6HexNAc4dHex1 | 0.00 | | | 0.00 | 0.00 | 0.28 |
| 2121 1A 0G | 7.18 | 0.00 | 2077.7 | Hex5HexNAc4dHex1NeuAc1 | 7.18 | 0.00 | | | 7.18 | 0.00 |
| 3111 1A 0G | 0.00 | 0.73 | 2118.8 | Hex4HexNAc5dHex1NeuAc1 | 0.00 | 0.73 | | 0.00 | 0.00 | 0.73 |
| 3130 0A 0G | 0.00 | 2.71 | 2151.8 | Hex6HexNAc5dHex1 | 0.00 | | | 0.00 | 0.00 | 2.71 |
| 2022 2A 0G | 1.39 | 0.00 | 2222.8 | Hex5HexNAc4NeuAc2 | 1.39 | | | | | |
| 2122 2A 0G | 1.00 | 0.00 | 2367.9 | Hex5HexNAc4dHex1NeuAc2 | 1.00 | 0.00 | | | 1.00 | 0.00 |
| 3131 1A 0G | 0.00 | 10.76 | 2442.9 | Hex6HexNAc5dHex1NeuAc1 | 0.00 | 10.76 | | 0.00 | 0.00 | 10.76 |

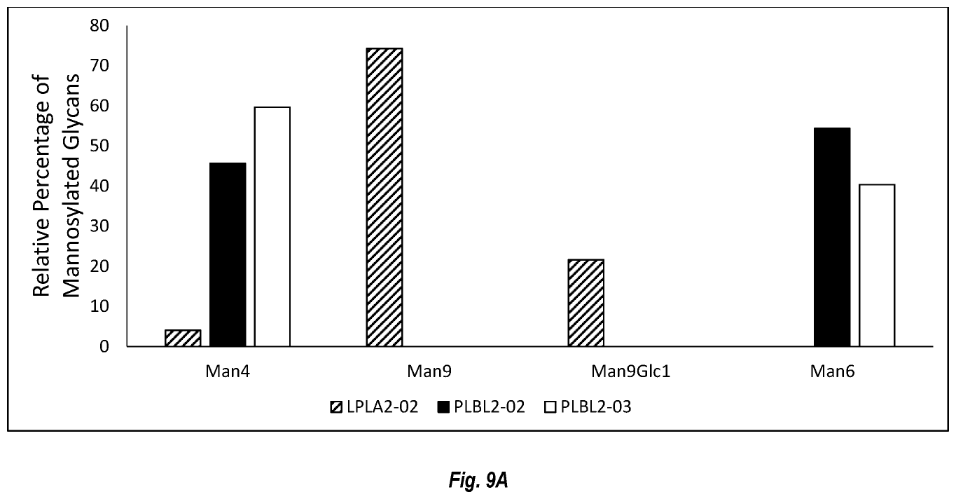

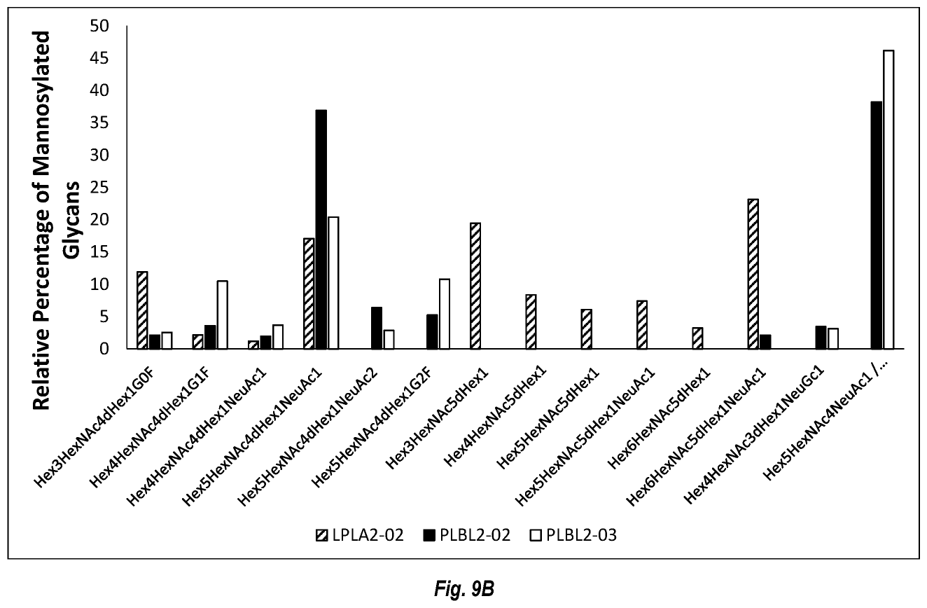

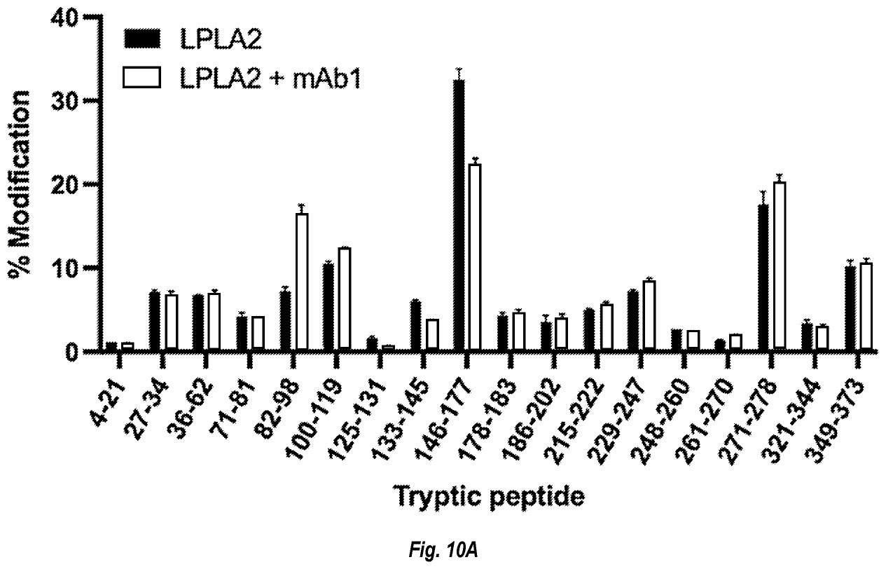

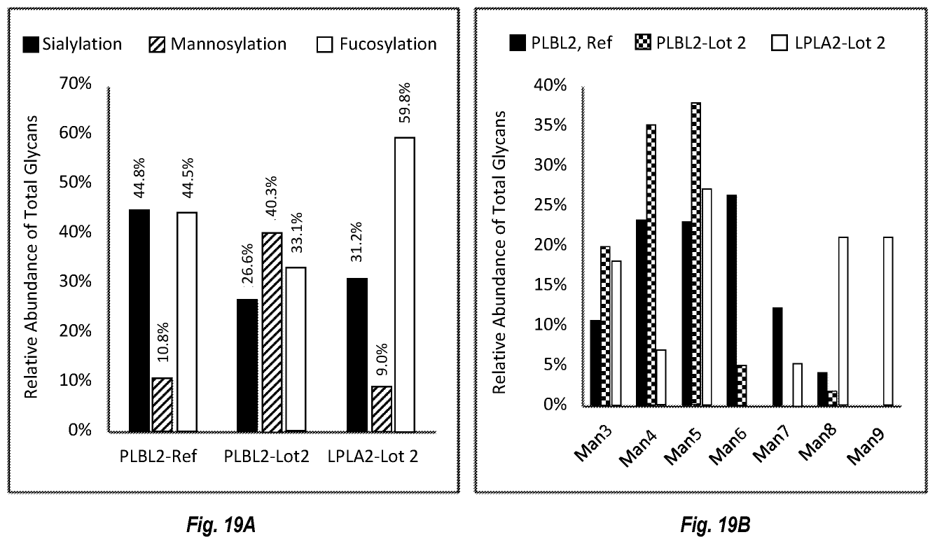

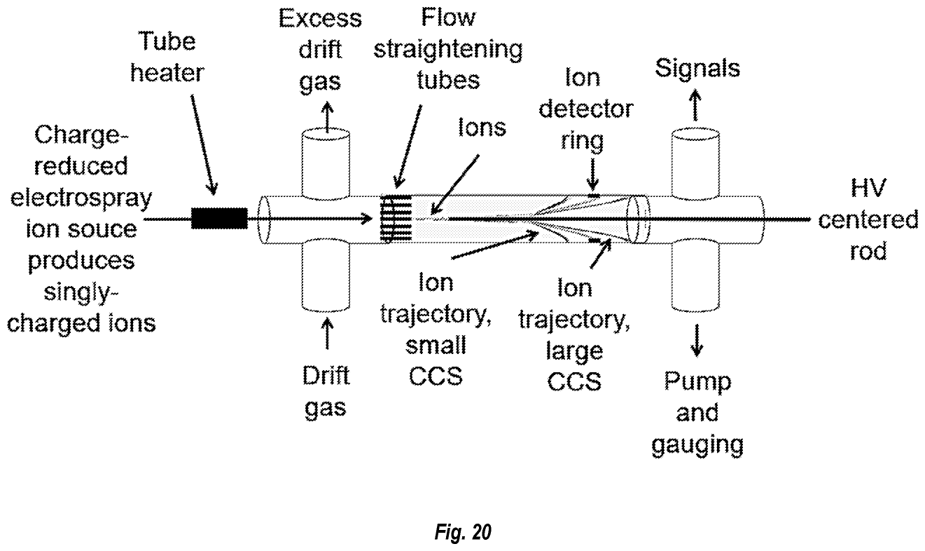

The compositions of the PNGase F-released glycans were assessed for trends that could explain the differences in lipase binding. (FIG. 19.) Compared to the reference, PLBL2-lot 2 (PLBL2-02) had ˜20% reduced sialylation, 30% increased mannosylation, and 10% decreased fucosylation. While there was insufficient sample remaining to analyze the released glycans from the LPLA2 reference, the LPLA2-lot 2 (LPLA2-02) sample, which had no binding activity, was predominantly composed of fucosylated species (60% relative abundance). As sialylation was known to be irrelevant to binding from the native MS binding experiments, the fucosylated and mannosylated glycans were compared (FIG. 9). There were not trends in fucosylation observed. While LPLA2-02 and PLBL2-01 had similar levels of total mannosylation, they differed in the size of the species detected, where LPLA2-02 contained nearly all Man9 or Man9Glc1 species, and PLBL2-01 contained only Man4 or Man6 species. Likewise, PLBL2-02 only contained the smaller mannose glycans Man4 and Man6. Compared to PLBL2, LPLA2-01 had smaller fucosylated species present and sampled approximately eight species at similar levels. PLBL2-01 and PLBL2-02 both had significant amounts of Hex5HexNAc4NeuAc1/Hex4HexNAc4dHex 1NeuGc1 (isobaric glycans) and Hex5HexNAc4dHex 1NeuAc1 present. 50% of LPLA2 lot 2's mannosylated species were >1200 Da (Man 7-9), whereas >80% of glycans in both PLBL2 samples were size Man6 or smaller. PLBL2-lot 2 had the highest percentage of small mannose species of the three samples.

Storage of the lipases in their purification buffers at 4° C. for six months resulted in the enrichment of certain glycoforms in solutions, with other species crashing out. In all cases, refrigerated lipases failed to bind to antibodies (storage of samples at −80° C. retained forms/activity), creating a pseudo “knock-out” experiment. At six months, the mannose species in each sample significantly decreased compared to pre-storage conditions (FIG. 7, Table 3). The levels of fucosylation and sialylation either remained approximately the same or increased proportionally to the reduction in mannosylation. The differences between the lot variants and the timecourse treatments, coupled to the changes observed in glycosylation, strongly speak for a role for glycosylation in mediating binding.

### Determination of the Antibody Binding Region for Lipases

Fast photochemical oxidation of proteins offered an opportunity to assess differences in the solvent exposed surface area (SASA) of proteins pre and post complexation. While FPOP cannot distinguish binding interfaces from binding-induced conformational changes, it provides regions that can be further interrogated for their precise role in binding (23). Each mAb-lipase pair was studied in two experiments wherein there was an excess of the mAb (10:1 molar ratio of mAb:lipase) to completely saturate the lipase and the converse with excess of lipase (1:10 mAb to lipase) in order to completely saturate the mAb binding site.

Peptides 125-131, 133-145 and 146-177 of LPLA2 displayed a decrease in oxidation upon complexation with mAb1 (FIG. 10A). This indicates that these peptides were protected from the solvent on complexation, and therefore may be involved in binding to mAb1 Upon incubation with excess mAb2, peptides 133-145, 146-177, 229-247 and 248-260 of LPLA2 displayed a decrease in oxidation indicating binding (FIG. 10B). Thus, peptides 133-145 and 146-177 of LPLA2 were common binding epitopes for both IgG4 isotype mAb1 and IgG1 isotype mAb2 while the other peptides were unique to each mAb respectively. In particular, peptide 146-177 of LPLA2 had a significant decrease in oxidation after complexation to both mAb1 and mAb2. (FIGS. 10A and 10B.) For PLBL2, peptides 67-78, 79-98, 173-187, 359-371, 372-388, 389-400, 401-407 and 424-459 of PLBL2 were implicated in binding to both mAb1 and mAb2, while peptides 211-236, 241-253, 287-333, 340-352, 513-530, 539-546, 573-599 and peptides 56-64, 485-512, 548-572 were interacting regions for mAb1 and mAb2 respectively (FIGS. 11A and B). In particular, peptides 79-98, 424-459, and 573-599 were common interacting regions with both mAb1 and mAb2, while peptides 372-388 and 548-572 only displayed reduced oxidation in complex with mAb2 and peptide 424-459 only displayed reduced oxidation in complex with mAb1.

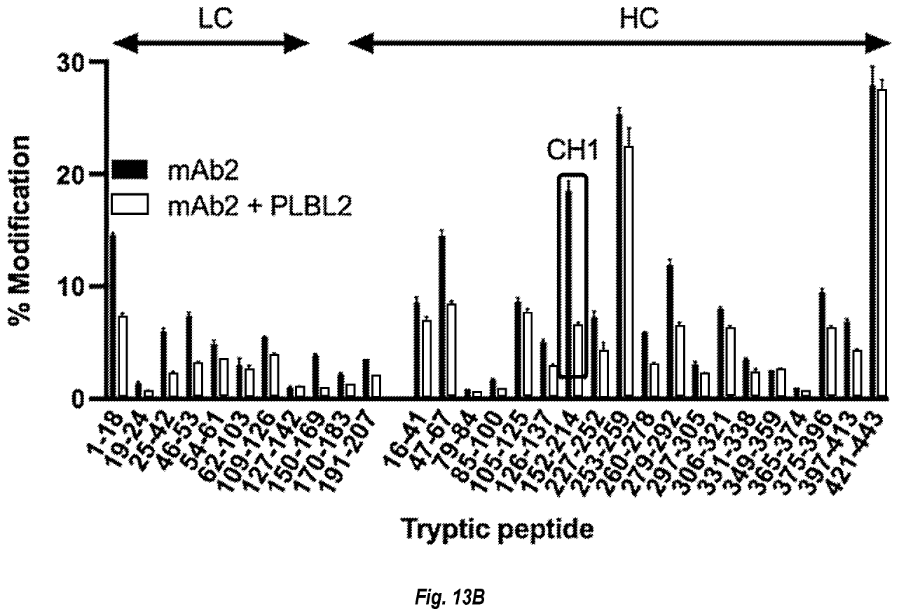

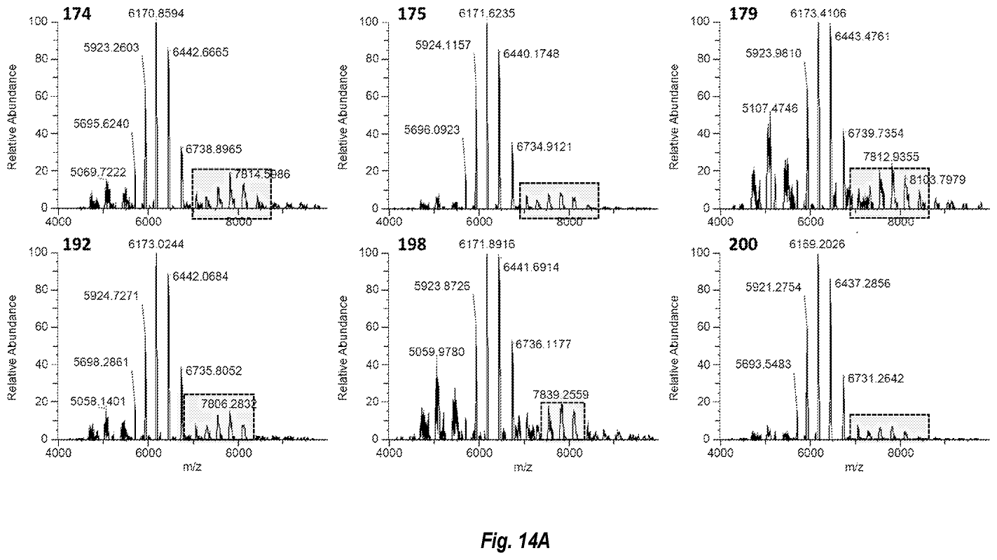

The converse experiment, with an excess of lipase, allowed identification of binding epitopes on the mAbs (FIGS. 12 and 13). When complexed to LPLA2, mAb1 peptides 131-146, 154-173, 174-187 of the light chain and peptides 6-38, 57-64, 76-122, 123-134, 149-197, 300-315 and 369-390 of the heavy chain displayed reduced oxidation upon complexation and thus, were implicated in LPLA2 binding. Similarly, mAb1 peptides 113-130, 131-146, 154-173, 195-211 of the light chain and 6-38, 57-64, 76-122, 149-197, 220-246, 254-286, 325-332, 343-358 of the heavy chain were implicated in binding to PLBL2. For the mAb2-LPLA2 complex, the mAb1 peptides involved in binding were 1-18 and 127-142 of the light chain and 46-67, 126-137, 152-214, 306-321, 331-338, 375-396, 397-413 of the heavy chain. For the mAb2-PLBL2 complex, the mAb1 peptides involved in binding were 150-169, of the light chain and 47-67, 85-100, 126-137, 152-214, 260-278, 279-292, 375-396, 397-413 of the heavy chain.

In particular, when complexed to LPLA2, mAb1 peptides 66-100 of the light chain (LC), and 6-38 of the heavy chain (HC) had significantly less oxidation against the control. For PLBL2, mAb1 LC peptides 50-65, 131-146, 154-173 and HC peptides 6-38 and 76-122 showed reduced oxidation. The HC peptide 149-197 was identified as a common mAb1 binding interface for LPLA2 and PLBL2. For mAb2 complexes, LC peptides 1-18 and 127-142 for LPLA2 or 150-169 for PLBL2 displayed a reduction in oxidation. For both lipases, LC peptides 25-42 and 46-53 and HC peptides 47-67 and 152-214 showed changes, which suggested changes due to binding. (See FIGS. 12 and 13.)

The oxidation changes in mAb1 peptides 149-197 and mAb2 peptides 152-214 suggested that a common binding interface fell on the mAb constant CH1 region. Since this region is largely conserved across different antibodies (including subtypes), we hypothesized that this interface could be a universal binding site for the diverse class of lipases produced by host cells and found across different drug products. To test if binding could be disrupted, single alanine mutations of the 32 CH1 domain residues of mAb1 were prepared and first screened against PLBL2 by SPR (Table 4). Fold decreases in K_{D} ranged from 0-70-fold, with 84% having at least a 5-fold effect.

TABLE 4
Screening of mAb1 alanine mutants against PLBL2 by SPR.

| | ka | kd | KD | Fold decrease in KD |
|---|---|---|---|---|
| mAb1 WT | 4.73E+03 | 0.01671 | 3.53E−06 | |
| G170A | 5.69E+02 | 0.009909 | 1.74E−05 | 5 |
| V171A | 2.04E+02 | 0.01245 | 6.12E−05 | 17 |
| T173A | 1.83E+02 | 0.009921 | 5.42E−05 | 15 |
| F174A | 1.53E+02 | 0.0378 | 2.47E−04 | 70 |
| P175A | 9.78E+01 | 0.01397 | 1.43E−04 | 40 |
| V177A | 2.45E+02 | 0.01264 | 5.17E−05 | 15 |
| L178A | 2.64E+02 | 0.01197 | 4.53E−05 | 13 |
| Q179A | 1.02E+02 | 0.02355 | 2.32E−04 | 66 |
| S180A | 2.62E+02 | 0.01583 | 6.04E−05 | 17 |
| S181A | 1.99E+02 | 0.01292 | 6.51E−05 | 18 |
| G182A | 1.47E+02 | 0.0143 | 9.71E−05 | 27 |
| Y184A | 6.60E+02 | 0.008499 | 1.29E−05 | 4 |
| L186A | 6.37E+02 | 0.01634 | 2.57E−05 | 7 |
| S187A | 1.09E+03 | 0.01262 | 1.16E−05 | 3 |
| F154A | 5.47E+02 | 0.01908 | 3.49E−05 | 10 |
| P155A | 4.60E+02 | 0.04123 | 8.96E−05 | 25 |
| V189A | 1.46E+02 | 0.01302 | 8.95E−05 | 25 |
| V190A | 1.71E+02 | 0.007244 | 4.25E−05 | 12 |
| T191A | 1.23E+02 | 0.00831 | 6.78E−05 | 19 |
| V192A | 1.48E+02 | 0.01682 | 1.14E−04 | 32 |
| P193A | 1.27E+02 | 0.007293 | 5.73E−05 | 16 |
| S194A | 3.59E+02 | 0.006978 | 1.94E−05 | 5 |
| S195A | 1.56E+02 | 0.006177 | 3.95E−05 | 11 |
| S196A | 1.58E+02 | 0.008049 | 5.11E−05 | 14 |
| L197A | 1.05E+03 | 0.01176 | 1.12E−05 | 3 |
| L198A | 1.88E+02 | 0.02024 | 1.08E−04 | 30 |
| T199A | 8.24E+02 | 0.007519 | 9.13E−06 | 3 |
| K200A | 1.12E+02 | 0.0129 | 1.16E−04 | 33 |
| E156A | 6.12E+04 | 0.09252 | 1.51E−06 | 0 |
| P157A | 4.03E+02 | 0.007351 | 1.82E−05 | 5 |
| V158A | 4.36E+02 | 0.007987 | 1.83E−05 | 5 |
| TI59A | 1.67E+02 | 0.008762 | 5.24E−05 | 15 |

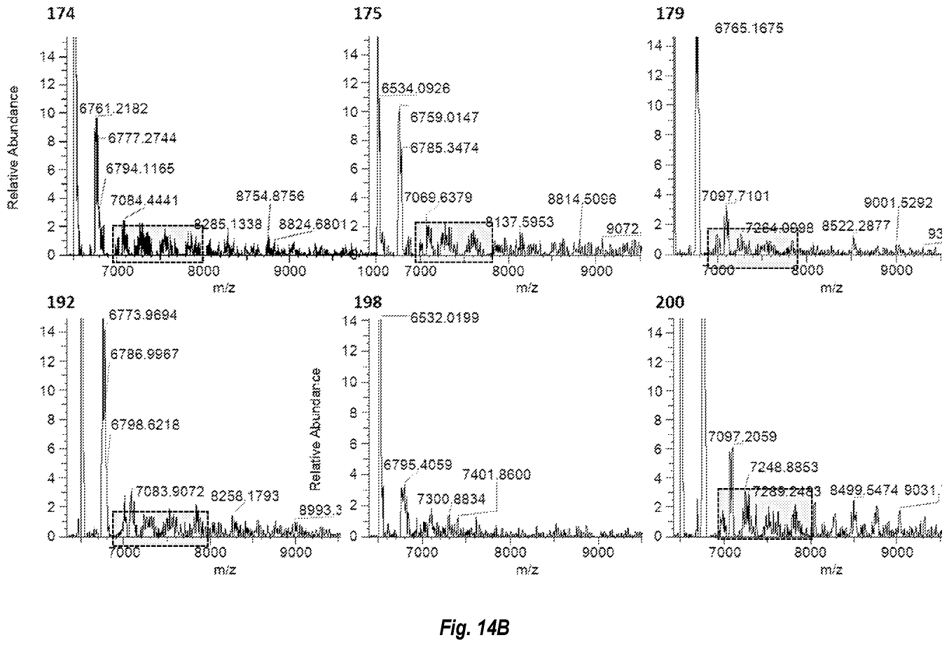

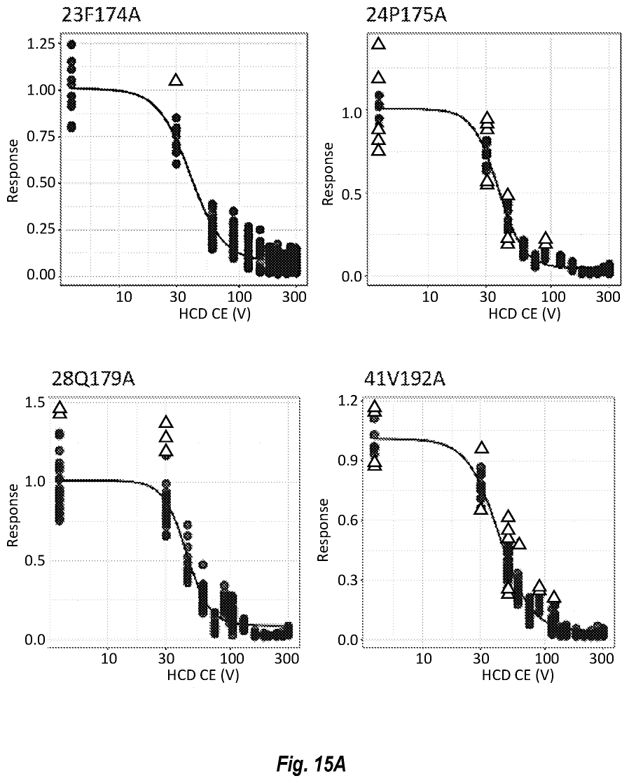

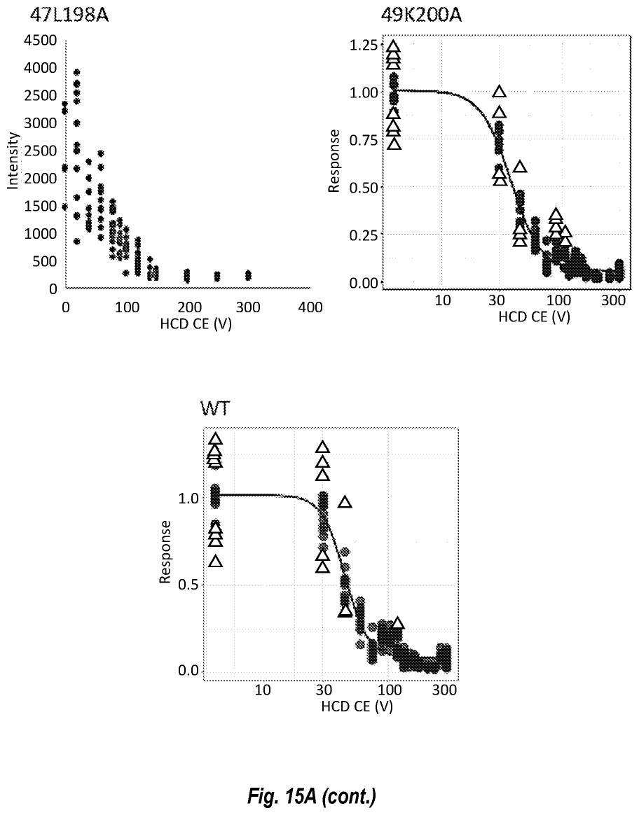

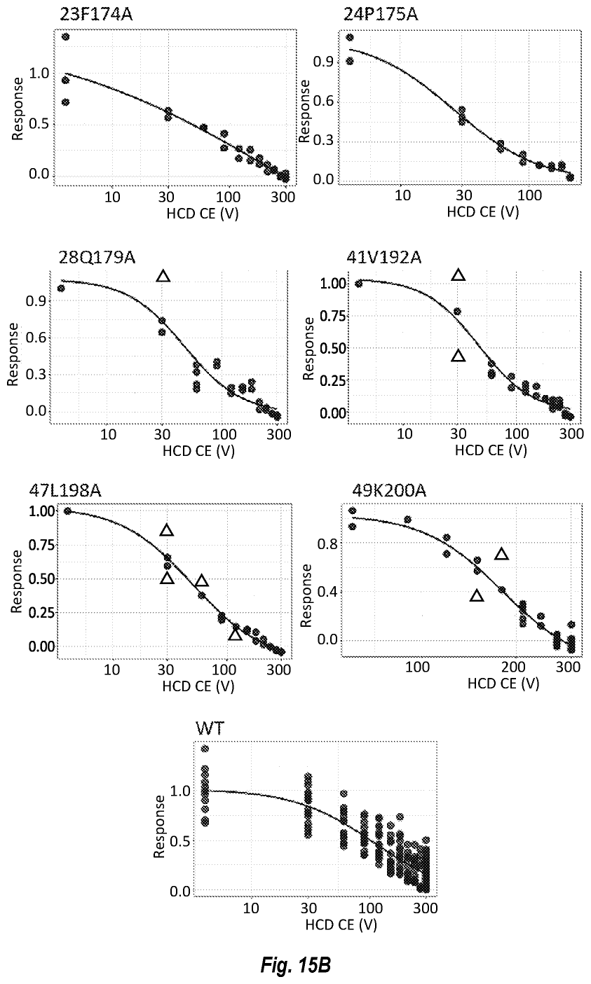

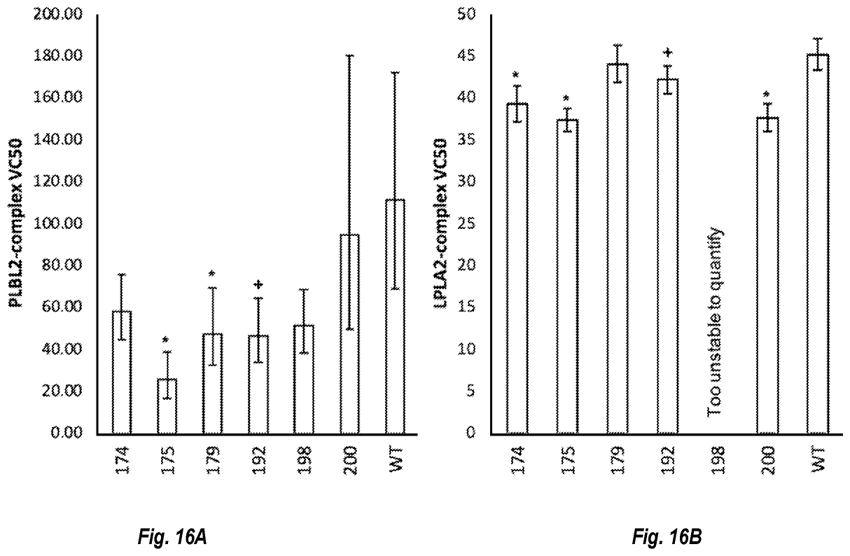

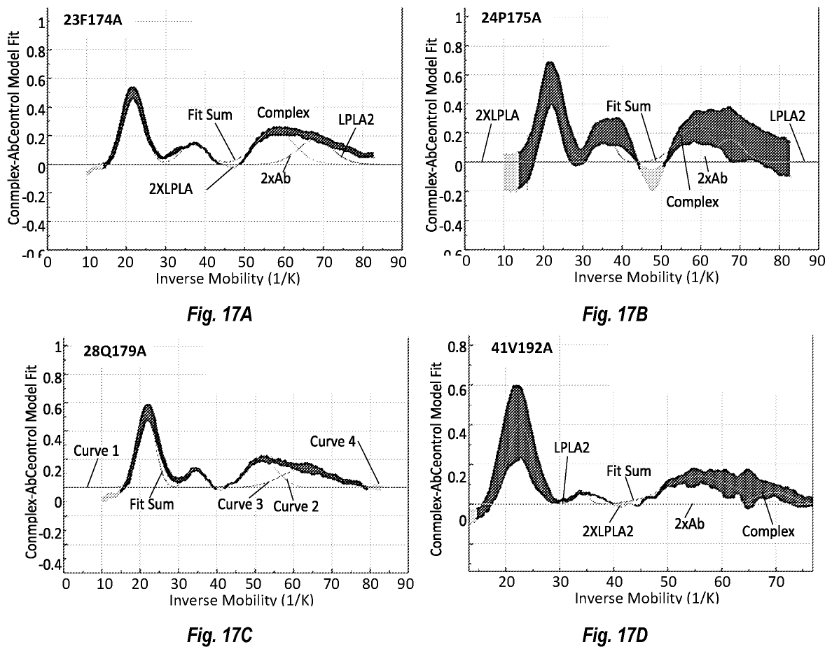

The subset of mutations that most substantially reduced binding (30-70 fold) were then tested in the LPLA2-mAb1 system by native MS and IM, because LPLA2 was not compatible with SPR. Compared to the stoichiometry and intact mass deconvolution experiments, the MS was re-tuned to preferentially increase the signal-to-noise of the complex peaks by changing to a lower resolving power (Table 5a). The +29 charge state of each LPLA2 or PLBL2-mAb1 complex was then isolated in the MS and subjected to a dissociation experiment to extrapolate their relative binding affinities to mAb WT, where the VC50 represents the level of HCD fragmentation energy to dissociate 50% of the complex (Table 5b, FIGS. 14-15). For mutants that knocked out binding such that the signal of the intact complex could not be measured (i.e. the lowest energy possible in the mass spectrometer fell on the S-curve slope), the VC50 is not reported (FIG. 15). PLBL2-02 was a significantly more stable binder than LPLA2-03 against WT mAb1, with a VC50 approximately 2.5 times higher and a MS1 S/N approximately 7-fold higher (112.6 versus 15.6), reflecting the differences in their relative binding affinities revealed by MST (Table 1). Therefore, per unit HCD fragmentation voltage, LPLA2-IgG4-B complexes would be expected to undergo increased dissociation compared to PLBL2. The decreases observed in the binding energy (VC50) of the mAb1 mutant species, compared to their respective wild types, against PLBL2 or LPLA2 showed similar trends (Table 5b. FIG. 16). Mutant L198A was too unstable for analysis when complexed to LPLA2, and likewise had over 50% reduced activity against PLBL2. Mutant P175A significantly decreased binding, with 95% confidence, against both lipases, with a 77% decrease in PLBL2 and a 17% decrease in LPLA2. While Q179A and V192A showed significantly decreased stability against PLBL2, there was no significant change in the Q179A VC50 against LPLA2, and a decrease of 6% for mutant V192A was only significant at the 90% confidence interval. Mutant K200A and F174A had a 16% and 13% decrease, respectively, in the VC50 versus the LPLA2-WT sample. No difference was observed for either species against PLBL2 complexes.

TABLE 5a
MS Parameters optimized for the detection of free lipase, free antibody,
lipase antibody complexes at 17,500 RP, and SIM experiments at 4375
RP (settings used for binding dissociation experiments).

| | Free | Free | Complex @ | SIM complex @ |
|---|---|---|---|---|
| | Lipase | Antibody | 17,500 RP | 4375 RP |
| Injection Time | 100 | 50 | 100 | 1000 |
| MS2 isolation | N/A | N/A | N/A | 20 |
| window | | | | |
| S-lens voltage | 21 | 21 | 21 | 21 |
| Skimmer voltage | 15 | 15 | 15 | 15 |
| In-Source | −50 | −150 | −50 | −10 |
| Trapping | | | | |
| Injection flatapole | 5 | 5 | 5 | 8 |
| offset (V) | | | | |
| Bent flatapole DC | 2 | 2 | 2 | 2 |
| Quadrupole | High | High | High | High |
| transmission RF | | | | |
| mode | | | | |
| Trans multipole | 0 | 0 | 0 | 0 |
| DC | | | | |
| C-trap entrance | 2 | 2 | 2 | 2 |
| lens | | | | |
| Orbitrap detector | Low | Low | Low | Low |
| mode | | | | |
| Extended | 10 | 3 | 0 | 3-300 |
| Trapping Voltage | | | | |
| Pressure | 4 | 1 | 6 | 1 |

TABLE 5b
VC50 values and confidence intervals (CI) for
binding dissociation native MS experiments
LPLA2-03 and PLBL2-03 against mAb1 mutants.

| | LPLA2 | | | PLBL2 | | |
|---|---|---|---|---|---|---|
| | | Lower CI, | Lower CI, | | Lower CI, | Lower CI, |
| Mutant | VC50 | VC50 | VC50 | VC50 | VC50 | VC50 |
| 174 | 39.28 | 37.23 | 41.44 | 58.18 | 44.71 | 75.71 |
| 175 | 37.4 | 36.04 | 38.82 | 25.49 | 16.72 | 38.87 |
| 179 | 44.05 | 41.91 | 46.3 | 47.29 | 32.2.8 | 69.3 |
| 192 | 42.21 | 40.59 | 43.88 | 46.63 | 33.83 | 64.27 |
| 198 | Too low to quantify | | | 51.18 | 38.23 | 68.52 |
| 200 | 37.65 | 36.01 | 39.36 | 94.52 | 49.56 | 180.28 |
| WT | 45.18 | 43.35 | 47.09 | 111.43 | 68.76 | 171.98 |

To validate the MS data and compare the relative concentration of complex formed. IM analysis was performed for LPLA2-mAb1 species (Table 6, FIG. 17). Mutant 47L198A had approximately a 90% decrease in the amount of complex formed compared to WT, corroborating the low-levels detected by native MS. Mutants 23F174A, 24P175A, and 28Q179A were decreased over 50% compared to the WT. Minor decreases of 36% and 12% were observed for mutants 41V192A and 49K200A.

TABLE 6
Ratio of the LPLA2-03-antibody complex peak areas, normalized against
the total IM spectral area, for each of the lipase mutants.

| | 174 | 175 | 179 | 192 | 198 | 200 | WT |
|---|---|---|---|---|---|---|---|
| Normalized | 0.63 | 0.55 | 0.54 | 0.88 | 0.18 | 1.21 | 1.38 |
| complex areas | | | | | | | |

The mass shift is reported against the experimentally determined deglycosylated intact mass (44491.5 Da). Where identifications were not possible due to the large combinatorial space of 14 different glycans permitted across four N-glycosylation sites, the relative glycan unit shifts compared to the most intense mass, labeled reference A, is given. Of 38 deconvolved masses, only three were not found to be associated with a predicted glycosylation pattern.

TABLE 7
The reduced heterogeneity of desiaiylated LPLA2
permitted deconvolution of the spectra.

| | | | Delta Mass to |
|---|---|---|---|
| | Average | | Deglycosylated |
| Identification | Mass | Intensity | Intact Protein |
| Ref A | 51919.13 | 2.87E+04 | 7427.63 |
| A + Hex | 52286.90 | 2.01E+04 | 7795.40 |
| A + 2Hex − HexNac | 52042.92 | 1.96E+04 | 7551.42 |
| A + 2Hex − 2HexNac | 51840.66 | 1.75E+04 | 7349.16 |
| A + 2HexNac − Hex | 52161.78 | 1.75E+04 | 7670.28 |
| A + 2Hex + HexNac | 52448.30 | 1.72E+04 | 7956.80 |
| A + 2Hex + 2HexNac | 52651.66 | 1.60E+04 | 8160.16 |
| A − 2Hex − HexNac − Fuc | 51245.45 | 1.54E+04 | 6753.95 |
| A + 3Hex − HexNac | 52204.03 | 1.52E+04 | 7712.53 |
| A − 2Hex + 2HexNac | 52000.66 | 1.49E+04 | 7509.16 |
| A + Hex | 52082.84 | 1.38E+04 | 7591.34 |
| A + 2Hex + HexNac + Fuc | 52594.37 | 1.27E+04 | 8102.87 |
| A − Hex − HexNac | 51554.44 | 1.25E+04 | 7062.94 |
| A + 3Hex + 2HexNac + Fuc | 52960.11 | 1.24E+04 | 8468.61 |
| A + HexNac | 52122.14 | 1.09E+04 | 7630.64 |
| A − Fuc | 51775.95 | 1.07E+04 | 7284.45 |
| A + 4Hex + Fuc | 51121.84 | 1.03E+04 | 6630.34 |
| A + 3Hex + 3HexNac | 52814.34 | 1.02E+04 | 8322.84 |
| A − 2HexNac + Hex | 51677.66 | 1.01E+04 | 7186.16 |
| A + 4Hex + 3HexNac + Fuc | 53324.30 | 1.00E+04 | 8832.80 |
| A + 4Hex | 52570.86 | 9.64E+03 | 8079.36 |
| A − 3Hex − 2HexNac − Fuc | 50878.42 | 8.97E+03 | 6386.92 |
| A − Hex | 51756.77 | 8.97E+03 | 7265.27 |
| A − 3Hex | 52408.32 | 8.72E+03 | 7916.82 |
| A − Hex − HexNac − Fuc | 51409.79 | 8.36E+03 | 6918.29 |
| A + 2Hex + HexNac + 2Fuc | 52736.13 | 7.83E+03 | 8244.63 |
| A + HexNac − Fuc | 51979.77 | 7.60E+03 | 7488.27 |
| A + 5Hex + 4HexNac + Fuc | 53690.83 | 6.68E+03 | 9199.33 |
| A − 3Hex + HexNac | 51636.53 | 6.44E+03 | 7145.03 |
| A − 3HexNac | 51312.33 | 6.14E+03 | 6820.83 |
| A − HexNac | 51716.11 | 5.84E+03 | 7224.61 |
| A − 4Hex − HexNac | 52772.68 | 5.62E+03 | 8281.18 |
| A + 2Hex + HexNac + 3Fuc | 52883.36 | 4.35E+03 | 8391.86 |
| | 56709.13 | 4.32E+03 | 12217.63 |
| A + 3Hex + 3HexNac + Fuc | 53160.52 | 4.07E+03 | 8669.02 |
| | 57375.51 | 3.36E+03 | 12884.01 |
| Hex3HexNAc4G0 & | 47458.06 | 1.82E+03 | 2966.56 |
| Hex4HexNAc5NeuAc1 | | | |
| | 47594.05 | 1.50E+03 | 3102.55 |

### Discussion

Hypothesis-informed testing of lipase-antibody interactions through targeted and next-generation methods offers new opportunities to observe low affinity host cell protein-antibody binding. In this study, the recombinant expression and purification of lipases allowed new methods to be developed for screening against multiple antibodies. Furthermore, we probed mechanistic aspects of antibody lipase complex formation and revealed a new role for lipase glycosylation mediating complexation and determined a common structural region located in the Ab constant heavy chain that impacts binding.

Traditional approaches to detect host cell protein impurities have been hindered by the large dynamic range of co-purified proteins. A variety of sophisticated two-dimensional techniques, including MS/2D-gel electrophoresis (9, 24) and 2D-LC-MS (25, 26) have proven most successful for detecting new impurities (27), including lipases such as clusterin, PLBL2 and lipoprotein lipase (LPL). While proteins detected in LC-MS assays represent a manufacturing concern due to their abundance, low-level impurities, such as LPLA2, can still have enzymatic activity. When investigated with targeted PRM assays, instead of a discovery LC-MS approach, LPLA2 was observed at less than 1 ppm levels, which was shown to be functionally relevant for the hydrolysis of polysorbate (11).

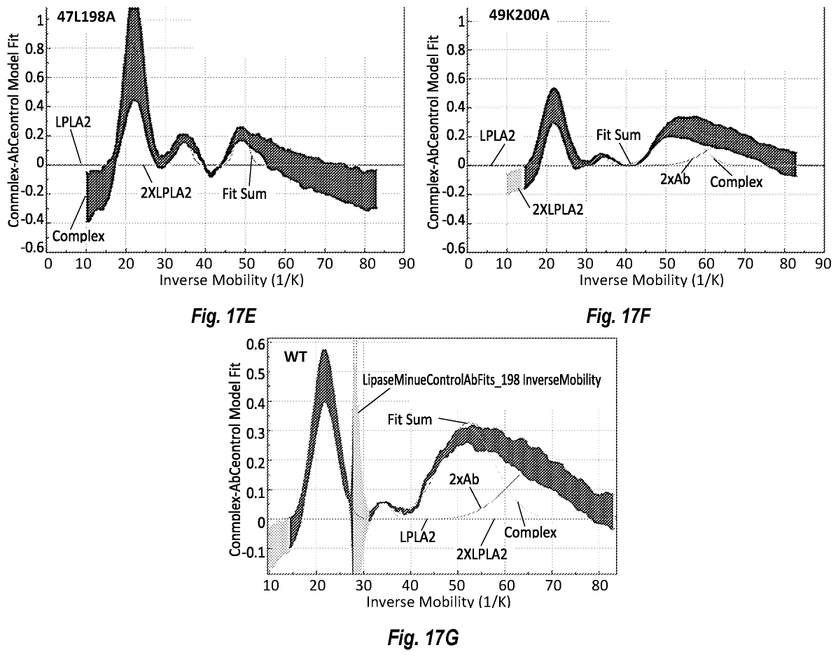

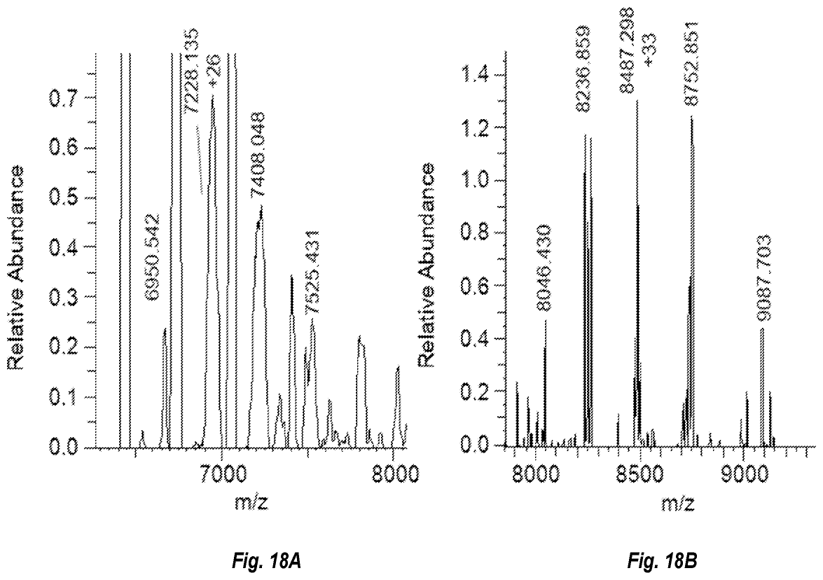

Selection of lipases for targeted analysis could be based on experimental proteomics datasets (28), the predicted proteome database (193 results for lipase search results in CHO cells in the TrEMBL database), and prior knowledge of lipase-antibody interactions across any system, rather than detection within a manufactured product. Based on this approach, three additional lipases, palmitoyl protein thioesterase (PPT), phospholipase D3 (PLD3), and sphingomyelin phosphodiesterase (SP) were also screened for binding by native MS (FIG. 18). SP was not detected, and PPT and PLD3 complexes could only be observed in 100 mM ammonium acetate, rather than 50 mM, suggesting that electrostatic repulsion could play a role in mediating binding. For PPT, found at 0.6% relative abundance, binding was only achieved at a 100:1 protein:mAb solution ratio (FIG. 18A), supporting prior work showing a concentration-dependent binding effect (29). For PLD3 (FIG. 18B), which exists naturally as a dimer, a 2:1 stoichiometric complex at 0.8% levels was observed in a 10:1 solution ratio. Binding was not observed between mAb1 mutants and PPT or PLD3, although a reduction in binding as small as 5% would likely have prevented detection due to the low abundances observed with the WT.

The detection of PPT and PLD3 complexes highlights the requirement for orthogonal and native solution techniques to characterize binding. Just as MST could screen for LPLA2 binding, which went undetected in SPR experiments, native MS observed PPT and PLD3 complexes that went undetected by MST. Differences between the assays may be caused by the immobilization or labeling of proteins leading to structural changes, or in the sensitivity of the detector, such as in the comparison of native MS to atmospheric pressure ion mobility. While native MS and FPOP experiments provide high level structural details, MST and atmospheric ion mobility are much more amenable to higher-throughput assays, positioning them to be a first-screen for mutant testing or knock-out experiments.

Early work to establish PLBL2-mAb1 binding was performed using SPR (9), which suggested a role for the F(ab′)2 domain, and these interactions were newly detailed in this study across multiple lipases and antibodies. With FPOP analysis, the SASA of specific regions on the lipase and mAbs were affected by binding, leading to significant decreases in the percent oxidation observed for the mAb CDR-L2, CDR-H1, and CDR-H3 regions. The CH1 domain was a common motif across antibodies implicated by FPOP, leading to a hypothesis that this was a specific interaction site for lipase binding. Conserved interactions may provide a baseline affinity for binding, while the antibody or lipase-specific binding regions may diminish or enhance affinity, accounting for different binding dissociation constants between a given lipase to different mAbs.

Mutagenesis across the CH1 region significantly diminished binding to LPLA2 and PLBL2, supporting the role of this region as structurally important to binding. The results of these alanine-scan experiments were consistent between SPR, ion mobility, and native MS. Protein A binding of antibodies is known to occur to the Fc portion of the antibody, between the CH2 and CH3 domains (30). Were lipase antibody complexes to form on-column, during purification, the F(ab′)2 domains would be the most solvent accessible region for lipase binding. The importance of antibody orientation on the beads in a chromatography column is supported in a study examining the impact of antibody load on protein A column, where it was shown that PLBL2 elution increased at a disproportionately greater rate to antibody load (31). The authors proposed that an increasing number of interaction sites on a column could be responsible, and the work reported here suggests that specifically, the F(ab′)2 domain orientation could be a critical parameter. Interestingly, the CH1 region tested in this study neighbors a region implicated in PLBL2 binding to mAbs, as determined by SPR, and reported in a 2018 PCT publication (32). However, this region did not appear as a common epitope across the lipases and mAbs that were tested in this study. Possible differences include each study's buffer composition or structural differences in the expressed lipases.

Lipase binding sites were also identified in the Fc region, although this region was not targeted for mutagenesis. The role of the Fc region in lipoprotein binding was first demonstrated for the glycoprotein clusterin (29, 33). In that work, papain-generated Fc and FAb fragments (derived from an IgG1 and IgG3 mixture) were shown to bind with similar affinity to IgG2 and IgM isotoypes, respectively, using an ELISA assay that showed the strongest binding to intact IgG1. Since then, the Fc has been implicated in PLBL2 binding (32, 34, 35) and LPL (36) to antibodies.

How lipase structure influences binding was evaluated by FPOP, MS and IM and then further investigated with exoglycosidase experiments to establish the effects of glycosylation. While deglycosylation inhibited complex formation, desialylation had no effect on binding by native MS. Were fucosylation to promote binding, then the second lot produced of LPLA2, which was 60% composed of fucosylated species, would have been expected to form mAb complexes; likewise, PLBL2-02, which had 10% fewer fucose species, bound tighter than PLBL2-01. Lipase binding activity therefore seemed to be most sensitive to the amount and type of mannose glycans. The tightest binders, PLBL2-01 and PLBL2-02 were enriched in low-number mannose glycans (Man3-5). These also correspond to the smallest molecular weight glycans, correlating the IM results that showed complexation of low molecular weight intact lipase glycoforms. Interestingly, the common epitopes found in the LPLA2 and PLBL2 samples overlap a glycosylation site, indicating that there may be one glycan sitting at the binding interface. We hypothesize that glycans that are too large could interfere with the protein-mAb orientation, but elimination of the glycans could remove hydrogen bonding partners.

While lipase expression during mAb production helps host cells survive, the results presented here indicate there is a possible opportunity to produce cell lines with partially deglycosylated lipases through asparagine-mutagenesis. Glycosylation-engineered lipases could minimize the formation of non-covalent mAb-lipase complexes and ultimately reduce the propensity for these types of enzymes to persist in formulation buffers, leading to visible particulates. Vice versa, mutagenesis of the CH1 domain, which does not play a central role in antigen binding, could limit co-purifying antibodies. Both strategies delineated here represent new opportunities for controlling HCP expression and purification in manufacturing, and remains an important avenue for further testing and exploration.

### Methods

### Materials

The therapeutic mAbs used in this study were produced at Genentech, Inc. Ammonium acetate (AMAC), formic acid (FA), dithiothreitol (DTT), guanidine HCl, methanol (MeOH), and tris HCl were purchased from Sigma-Aldrich (St Louis, Mo.). Acetonitrile (ACN), trifluoroacetic acid (TFA) and water was purchased from Fisher Scientific (Hampton, N.H.). All solvents were HPLC grade or >99.9% purity.

### Protein and Antibody Expression

Plasmids were made by Genewiz Inc. (South Plainfield, N.J.) through gene synthesis and subcloning. All lipases, mAbs and alanine mutants were expressed in CHO cells. The lipases were purified by affinity chromatography using a Ni NTA column (Cytiva, Marlborough, Mass.) followed by gel filtration chromatography. mAbs were purified using a protein A column (Cytiva, Marlborough, Mass.) followed by gel filtration chromatography. Proteins were characterized using SDS-PAGE and analytical SEC.

The following notation is used for proteins generated at different time points in multiple batches, with their associated production dates given: PLBL2-01 (November 2020), PLBL2-02=(batch cr3 Nov. 2020), LPLA2-01=February 2019, LPLA2-02=November 2020, LPLA2-03=June 2020.

### Surface Plasmon Resonance Analysis

Lipases of interest were immobilized onto CMS chip via amine coupling. Experiments were carried out on a Biacore® T200 Instrument (Uppsala, Sweden) using PBS as the running buffer and 10 mM Glycine pH 2.0 as the regeneration buffer. At least 8 different concentrations of the mAb were injected onto the chip immobilized with the lipase and the binding curves were globally fit to the 1:1 Langmuir binding model.

### Microscale Thermophoresis (MST)

In order to measure binding using MST, the lipase was labeled using the Red-tris-NTA dye (NanoTemper Technologies Inc., South San Francisco, Calif.) that binds to the his-tag on the lipase. In brief, excess dye was incubated with the lipase and the labeled lipase was purified using a Zeba Spin desalting column with a 7 k molecular weight cutoff (Thermo Fisher Scientific, Waltham, Mass.). 1-5 nM of the labeled lipase was incubated with various concentrations of the mAb and subjected to thennophoresis. Data was analyzed using the MO.Screening Analysis Software (NanoTemper Technologies Inc., South San Francisco, Calif.).

### Fast Photochemical Oxidation of Proteins (FPOP)

Antibody:lipase solutions were prepared at a 1:10 or a 10:1 ratio. An arginine radical scavenger was added to the solutions before asymmetrically mixing with hydrogen peroxide as described previously (37). Samples were flowed through a 150 μm capillary and exposed to a 248 nm KrF excimer laser (GAM Laser Inc. Orlando, Fla.) pulsed at 30 mJ/pulse. The samples were collected in 10 μL of 50 nM catalase and 200 mM methionine to scavenge residual peroxide. Proteins were cleaned up using a molecular weight cut off filter, reduced, alkylated, and tryptically digested. Peptides were loaded onto an Agilent 1200 HPLC with a Waters BEH300 C18 (1.7 μm 2.1×150 mm) column. A flow rate of 0.3 mL/min was used, with solvent B (acetonitrile, 0.8% trifluoroacetic acid) increased to 55% at 45 min. Peptides were detected on a Orbitrap™ Elite (Thermo Fisher, Bremen, Germany) in full scan positive-ion mode at 60,000 resolving power in data-dependent acquisition mode. Peak identification and quantitation of percent oxidation for each peptide were performed using Byos® Software Suite (Protein Metric Inc., Cupertino, Calif.). Spectra was searched against peptides that were identified using Mascot with a custom database (including a decoy database) using the antibodies or lipases of interest. All oxidation-based modifications were enabled as variable modifications, and the mass tolerance was set at 10 ppm. The modification intensities were taken from the extracted ion chromatogram of the peptides at the MS1 level.

### Desalting and Preparation of Protein Samples for MS and IM

Protein or antibody samples were buffer exchanged into 50 mM ammonium acetate (pH 7) and exchanged according to the manufacturer protocol on a Micro Bio-Spin™ 6 column (Bio-Rad, Hercules, Calif.). Samples were used within three days of desalting and stored at 4° C. deg. Desalted samples were then split for native MS and IM analyses. Complex was prepared for MS analysis at a 10:1 lipase:antibody (2.7:0.27 μM) molar ratio, respectively, and for IM at a 2:1 molar ratio (400:200 nM), respectively, just prior to analysis, at a 50 mM and 25 mM final ammonium acetate concentration, respectively.

Treatment of Lipases with Glycosidases

For desialylation, lipases were incubated with α2-3,6,8 neuraminidase (New England Biolabs, Ipswich, Mass.) at 100 units/40 μg lipase for 3h at 37° C. for 3h. For native deglycosylation, samples were incubated with glycerol-free PNGaseF at 1 unit/5 μg lipase overnight at 37° C.

### Native Mass Spectrometry Analysis

Borosilicate glass (1.2 mm OD, 0.69 mm ID) was pulled on a P-1000 puller (Sutter Instruments, Novato, Calif.) using methods previously described (38). Tips were sputter coated to 6 nm with 80:20 Au/Pd using an Ace600 high vacuum sputter coater (Leica Microsystems Inc. Buffalo Grove, Ill.). Between 2-5 μL of sample was loaded into each tip, inserted into a Nanospray Flex source, and interfaced to a Q Exactive™ UHMR (Thermo Fisher Scientific, Bremen, DE). The capillary voltage was set between 1.2-1.3 kV to maintain stable spray and the inlet temperature was set to 200° C. MS transmission and detection conditions were optimized using approaches previously described (39). The final conditions for the control samples (free lipase, free antibody) and for low and high resolution complex spectra are reported in Table 5a. All spectra were deconvolved for analysis using UniDec 3.1 (40).

For mass spectrometry binding energy dissociation experiments, each +29 protein complex was isolated using the centroided peak m/z and a 20 m/z isolation window. The HCD collision energy voltage was swept from 3-300 V using a fixed injection time. An in-house python program was used to auto-extract the base peak intensity of peaks inside the isolation window and the associated HCD energies. These values were subsequently imported into R Studio v1.3 and fit with the dr4p1 package (41). The data was normalized, cleaned for outliers based on the Tukey method, and fit with a four-parameter logistic growth function using the Mead method for initial parameter selection, Broyden-Fletcher-Goldfarb-Shanno (BFGS) method for parameter optimization and eigenvalue selection through the computation Hessian method. VC50 values, taken as the voltage to reduce the complex to 50% of its initial intensity, were extracted and the areas under each curve were integrated.

### Atmospheric Ion Mobility Analysis

A non-MS, stand alone atmospheric ion mobility device, the IMgenius™ (IonDX, Inc.) was used to compare the formation complex between different antibodies and antibody mutants. The IMgenius (Figure A6), which has yet to be described in the literature and is based on work to measure the particle sizes of lipoproteins (22), separates singly charged, electrosprayed ions in an electric field according to their collisional cross sectional area. Samples were infused at 300 nL/min using a nanoLC system adapted for flow injection and equipped with pacified fused silica capillary (220 μm OD, 50 μm ID). Electrospray onset was at 2.7-3 kV in a chamber with 1.9 SLM air and 0.1 SLM CO₂. The central rod voltage was swept from 0 to 4 kV and the current detected on a 3 mm wide ring digitized with a 4-channel 12-bit Pico-Scope (Model 4424, Pico Technologies, UK). Fluid dynamic models of the trajectory of singly charged ions, generated in SIMION (Scientific Instrument Services, Inc., Ringoes, N.J.), were used to construct a voltage versus mobility lookup table.

Spectra acquired were the average of five scans, background subtracted, and smoothed with a three-point moving average. For control data sets, data was normalized and imported Magicplot Pro 2.9.3 (Sydney, AU) (42). For complex protein data sets, the normalized antibody control IM spectra was subtracted from the normalized complex protein spectra. Control spectra were fit using an automated fit-sum approach of two or four Gaussian-A curves (y(x)=a*exp(−ln(2)*(x−x0)\hat{ }/dx\hat{ }2)) for control or complex datasets, respectively, from which the mean inverse mobility and curve area were exported. The Mason-Schamp equation was used to convert inverse mobility to particle diameter using an assumption of a spherical particle.

### Global N-Linked Glycan Composition Analysis by LC MS Analysis

Ten μg of protein was denatured with 8 M guanidine HCl at a 1:1 volume ratio and reduced with 100 mM dithiothreitol for 10 min at 95° C. Samples were diluted with 100 mM Tris HCl, pH 7.5 to a final concentration of 2 M guanidine HCl, followed by a 18-hour digestion at 37° C. with 2 μl of glycerol-free PNGase F (New England BioLabs, Ipswich, Mass.). Deglycosylated sample (150 ng) was injected onto a 1260 Infinity HPLC-Chip Cube, equipped with a 43 mm PGC-Chip II column (Agilent Technologies, Santa Clara, Calif.). A binary pump was used to deliver 500 nL/min solvent A (99.88% water, 0.1% formic acid and 0.02% trifluoroacetic acid) and solvent B (90% acetonitrile, 9.88% water, 0.1% formic acid and 0.02% trifluoroacetic acid) as a gradient of 2% to 32% B over 6 min, 32% B for 1.5 min, 32 to 85% over 0.5 min, and 85% B for 1 min. The column was re-equilibrated at 2% B for 3 min.

Glycans were electrosprayed into an Agilent 6520 Q-TOF mass spectrometer using the following parameters: 1.9 kV spray voltage; 325° C. gas temperature: 5 l/min drying gas flow; 160 V fragmentor voltage; 65 V skimmer voltage; 750 V oct 1 RF Vpp voltage; 400 to 3,000 m/z scan range; positive polarity; MS centroid data acquisition using extended dynamic range (2 GHz) instrument mode; 3 spectra/s: 333.3 ms/spectrum: 3243 transients/spectrum; and a CE setting of 0.

Acquired data were searched against a glycan library in the Agilent MassHunter Qualitative Analysis software. The software algorithm utilized a combination of accurate mass with a mass tolerance of 10 ppm and expected retention time for glycan identification. The AUC of extracted N-glycans was calculated, and the relative percentages, compared to the total glycan area per run, was determined.

### Example 2

Testing Complex Formation of Lipases with Deglycosylated Antibodies

Each antibody is prepared as follows −20-150 pg of antibody is diluted to a I pg/mL with glycobuffer 2 (diluted from 10×) and 20 mM ammonium acetate. Glycerol-free PNGas F (New England 15 Biolabs) is added to the antibody at a PNGase F:antibody ratio of 1:5. A control sample is prepared as follows −20-150 pg of antibody is diluted to a 1 μg/mL with glycobuffer 2 (diluted from 1Ox) and 20 mM ammonium acetate. No PNGase F is added.

Each antibody and control sample are incubated at 37° C. for 16 hours.

500 μL if 50 mM ammonium acetate is added to a 100 kDa molecular weight cutoff centrifugal filter (Amicon Ultra). The centrifugal filter is centrifuged at 25° C. for 10 minutes at 14,000×g. The flow through is discarded.

Each antibody and control sample is added to its own centrifugal filter. Up to 500 μL of antibody or control is added to each centrifugal filter. The centrifugal filters are centrifuged at 25° C. for 10 minutes at 14,000×g. The flow through is discarded. These centrifugation steps are performed three more times to concentrate the antibody and control samples.

Each concentrated antibody and control is collected. Each centrifugal filter is placed upside down in a new vial. Each centrifugal filter is centrifuged at 25° C. for 2 minutes at 1,000×g. The flow through is discarded. The filters are discarded, and the vials are closed and sealed with parafilm.

The concentration of antibodies in each antibody and control sample is determined using a Bradford assay.

For measurements by static spray native MS, antibody-complexes are prepared at an antibody:complex molar ratio of 1:10. For measurements by LC- MS, antibody-complexes are prepared at an antibody:complex molar ratio of 1:2. For measurements by ion mobility, antibody-complexes are prepared at an antibody:complex molar ratio of 1:1. Comparisons are made between the amount of

complexes formed by lipase and the control antibody, and the amount of complexes formed by lipase and the deglycosylated antibody.

### REFERENCES CITED IN THE EXAMPLES

1. M. Vanderlaan et al., Experience with host cell protein impurities in biopharmaceuticals. Biotechnology Progress 34, 828-837 (2018).
2. M. Jones et al., “High-risk” host cell proteins (HCPs): A multi-company collaborative view. Biotechnol. Bioeng. 118, 2870-2885 (2021).
3. S. K. Fischer et al., Specific Immune Response to Phospholipase B-Like 2 Protein, a Host Cell Impurity in Lebrikizumab Clinical Material. The AAPS Journal 19, 254-263 (2017).
4. N. A. Hanania et al, Lebrikizumab in moderate-to-severe asthma: pooled data from two randomised placebo-controlled studies. Thorax 70, 748-756 (2015).
5. S. X. Gao et al., Fragmentation of a highly purified monoclonal antibody attributed to residual CHO cell protease activity. Biotechnol. Bioeng. 108, 977-982 (2011).
6. N. Dixit, N. Salamat-Miller, P. A. Salinas, K. D. Taylor, S. K. Basu, Residual Host Cell Protein Promotes Polysorbate 20 Degradation in a Sulfatase Drug Product Leading to Free Fatty Acid Particles. J. Pharm. Sci. 105, 1657-1666 (2016).
7. F. Robert et al, Degradation of an Fe-fusion recombinant protein by host cell proteases: Identification of a CHO cathepsin D protease. Biotechnol Bioeng. 104, 1132-1141 (2009).
8. Test Procedures and Acceptance Criteria for Biotechnological Biological Products (Food and Drug Administration, US Department of Health and Human Services, Rockville, Md., 1999), vol. Q6B.
9. M. Vanderlaan, W. Sandoval, P. Liu. Hamster phospholipase B-like 2 (PLBL2), a host cell protein impurity in CHO-derived therapeutic monoclonal antibodies. BioProcess Int 13, 18-29 (2015).
10. J. Chiu et al., Knockout of a difficult-to-remove CHO host cell protein, lipoprotein lipase, for improved polysorbate stability in monoclonal antibody formulations. Biotechnol. Bioeng. 114, 1006-1015 (2017).
11. T. Hall, S. L. Sandefur, C. C. Frye, T. L. Tuley, L. Huang, Polysorbates 20 and 80 Degradation by Group XV Lysosomal Phospholipase A2 Isomer X1 in Monoclonal Antibody Formulations. J. Pharm. Sci. 105, 1633-1642 (2016).
12. M. Dwivedi, M. Blech, I. Presser, P. Garidel, Polysorbate degradation in biotherapeutic formulations: Identification and discussion of current root causes. Int. J. Pharm. 552, 422-436 (2018).
13. J. Zhu-Shimoni et al., Host cell protein testing by ELISAs and the use of orthogonal methods. Biotechnol. Bioeng. 111, 2367-2379 (2014).
14. S. Zhang et al., Putative Phospholipase B-Like 2 is Not Responsible for Polysorbate Degradation in Monoclonal Antibody Drug Products. J. Pharm. Sci. 109, 2710-2718 (2020).
15. S. Cao, N. Martinez-Martin, Unbiased Identification of Extracellular Protein-Protein Interactions for Drug Target and Biologic Drug Discovery [Online First]. IntechOpen 10.5772/intechopen.97310 (2021).
16. S. Zhang, H. Xiao. R. Molden, H. Qiu, N. Li, Rapid Polysorbate 80 Degradation by Liver Carboxylesterase in a Monoclonal Antibody Formulated Drug Substance at Early Stage Development. J. Pharm. Sci. 109, 3300-3307 (2020).
17. M. Thou, Q. Li, R. Wang, Current Experimental Methods for Characterizing Protein-Protein Interactions. ChemMedChem 11, 738-756 (2016).
18. V. Kairys, L. Baranauskiene, M. Kazlauskiene, D. Matulis. E. Kazlauskas, Binding affinity in drug design: experimental and computational techniques. Expert Opinion on Drug Discovery 14, 755-768 (2019).
19. A. Khodabandehloo, D. D. Y. Chen. Particle sizing methods for the detection of protein aggregates in biopharmaceuticals. Bioanalysis 9, 313-326 (2017).
20. W. Wang, C. J. Roberts, Protein aggregation—Mechanisms, detection, and control. Int. J. Pharm. 550, 251-268 (2018).
21. D. Hambly, M. Gross. Laser flash photochemical oxidation to locate heme binding and conformational changes in myroglobin. Int. J. Mass spectrom. 259, 124-129 (2007).
22. M. P. Caulfield et al., Direct Determination of Lipoprotein Particle Sizes and Concentrations by Ion Mobility Analysis. Clin. Chem. 54, 1307-1316 (2008).
23. K. S. Li, L. Q. Shi, M. L. Gross, Mass Spectrometry-Based Fast Photochemical Oxidation of Proteins (FPOP) for Higher Order Structure Characterization. Acc. Chem. Res. 51, 736-744 (2018).
24. A. K. Hunter et al., Separation of product associating E. coli host cell proteins OppA and DppA from recombinant apolipoprotein A-IMilano in an industrial HIC unit operation. Biotechnology Progress 25, 446-453 (2009).
25. C. E. Doneanu et al., Analysis of host-cell proteins in biotherapeutic proteins by comprehensive online two-dimensional liquid chromatography/mass spectrometry. Mabs-Austin 4, 24-44 (2012).
26. X. Liu et al., Identification and characterization of co-purifying CHO host cell proteins in monoclonal antibody purification process. J. Pharm. Biomed. Anal. 174, 500-508 (2019).
27. K. N. Valente, A. M. Lenhoff, K. H. Lee. Expression of difficult-to-remove host cell protein impurities during extended Chinese hamster ovary cell culture and their impact on continuous bioprocessing. Biotechnol. Bioeng. 112, 1232-1242 (2015).
28. D. Baycin-Hizal et al., Proteomic Analysis of Chinese Hamster Ovary Cells. J. Proteome Res. 11, 5265-5276 (2012).
29. M. R. Wilson, S. B. Easterbrook-Smith, Clusterin binds by a multivalent mechanism to the Fc and Fab regions of IgG. Biochim. Biophys. Acta 1159, 319-326 (1992).
30. J. Deisenhofer, Crystallographic refinement and atomic models of a human Fc fragment and its complex with fragment B of protein A from Staphylococcus aureus at 2.9- and 2.8-.ANG. resolution. Biochemistry 20, 2361-2370 (1981).
31. B. Tran et al., Investigating interactions between phospholipase B-Like 2 and antibodies during Protein A chromatography. Journal of Chromatography A 1438, 31-38 (2016).
32. H. Bosteels et al., Antibodies with reduced binding to process impurities (USPTO, WO2018065389, 2017).
33. M. R Wilson, P. J. Roeth, S. B. Easterbrook-Smith, Clusterin enhances the formation of insoluble immune complexes. Biochem. Biophys. Res. Commun. 177, 985-990 (1991).
34. C. Dumet, J. Pottier, V. Gouilleux-Gruart, H. Watier, Insights into the IgG heavy chain engineering patent landscape as applied to IgG4 antibody development. Mabs-Austin 11, 1341-1350 (2019).
35. F. Gunawan et al., Compositions and methods for detecting and quantifying host cell protein in cell lines and recombinant polypeptide products. (USPTO WO2015038884A2, 2016).
36. S. K. Singh, A. Mishra, D. Yadav, N. Budholiya, A. S. Rathore, Understanding the mechanism of copurification of “difficult to remove” host cell proteins in rituximab biosimilar products. Biotechnology Progress 36, e2936 (2020).
37. N. K. Garcia, A. Sreedhara, G. Deperalta, A. T. Wecksler, Optimizing Hydroxyl Radical Footprinting Analysis of Biotherapeutics Using Internal Standard Dosimetry. Journal of the American Society for Mass Spectrometry 31, 1563-1571 (2020).
38. N. Kirshenbaum, I. Michaelevski, M. Sharon, Analyzing large protein complexes by structural mass spectrometry. J Vis Exp 10.3791/1954, 1954 (2010).
39. M. van de Waterbeemd et al., High-fidelity mass analysis unveils heterogeneity in intact ribosomal particles. Nat. Methods 14, 283-286 (2017).
40. M. T. Marty et al., Bayesian deconvolution of mass and ion mobility spectra: from binary interactions to polydisperse ensembles. Anal. Chem. 87, 4370-4376 (2015).
41. H. An, J. T. Landis, A. G. Bailey, J. S. Marron, D. P. Dittmer, dr4p1: A Stable Convergence Algorithm for the 4 Parameter Logistic Model. The R Journal 11 (2019).
42. J. Ooi, D. Traini, P. M. Young, Graphing software for medical writers. Medical Writing 23, 41-44 (2014).
43. K. Lakomek et al., Initial insight into the function of the lysosomal 66.3 kDa protein from mouse by means of X-ray crystallography. BMC Struct. Biol. 9, 1-17 (2009).
44. A. Glukhova et al., Structure and function of lysosomal phospholipase A2 and lecithin:cholesterol acyltransferase. Nat. Commun. 6, 1-12 (2014).

1. A recombinant antibody produced by a host cell engineered to express said antibody, wherein said antibody has a modification (substitution, deletion, or addition) of at least one amino acid residue in the heavy chain CH1 region, CH2 region, or CH3 region of the antibody, wherein the modification results in altered interaction of the antibody with one or more endogenous lipases expressed by said host cell.

2. (canceled)

3. (canceled)

4. (canceled)

5. (canceled)

6. (canceled)

7. The antibody of claim 1, wherein the antibody comprises a human IgG4 constant region, and wherein said modification is a substitution of at least one amino acid from P149 to S197 (Kabat numbering).

8. The antibody of claim 1, wherein the antibody comprises a human IgG1 constant region, and wherein said modification is a substitution of an amino acid selected from V152 to P214 (Kabat numbering).

9. The antibody of claim 1, wherein said modification comprises a substitution of at least one amino acid selected from the group consisting of G170, V171, T173, F174, P175, V177, L178, Q179, S180, S181, G182, L186, F154, P155, V189, V190, T191, V192, P193, S194, S195, S196, L198, K200, P157, V158, and T159 (Kabat numbering).

10. The antibody of claim 1, wherein said modification comprises a substitution of an amino acid selected from the group consisting of F174, P175, Q179, V192, L198 and K200 (Kabat numbering).

11. The antibody of claim 9, wherein said substitution is selected from the group consisting of G170A, V171A, T173A, F174A, P175A, V177A, L178A, Q179A, S180A, S181A, G182A, L186A, F154A, P155A, V189A, V190A, T191A, V192A, P193A, S194A, S195A, S196A, L198A, K200A, P157A, V158A, and T159A (Kabat numbering).

12. The antibody of claim 11, wherein said substitution is selected from the group consisting of F174A, P175A, Q179A, V192A, L198A and K200A.

13. (canceled)

14. (canceled)

15. (canceled)

16. (canceled)

17. (canceled)

18. (canceled)

19. (canceled)

20. The antibody of claim 1, wherein said antibody comprises modifications in one, two, three, or four amino acid in the CH1 region.

21. (canceled)

22. (canceled)

23. The antibody of claim 1, wherein said antibody comprises modifications in four or more amino acids in the CH1 region.

24. (canceled)

25. A pharmaceutical composition comprising the antibody of claim 1.

26. An isolated nucleic acid encoding an antibody of claim 1.

27. An expression vector comprising the isolated nucleic acid of claim 26.

28. An isolated host cell containing, transformed or transduced with the expression vector of claim 27.

29. A method of treating a human subject in need thereof, comprising administering to said subject a pharmaceutically effective dose of the pharmaceutical composition of claim 25.

30. A method of producing the antibody of claim 1, comprising incubating a host cell containing, transformed or transduced with an isolated nucleic acid that expresses the antibody under conditions in which the antibody is produced.

31. (canceled)

32. A method of reducing interactions between a recombinant antibody and one or more lipases expressed in a host cell used to express the antibody, wherein the method comprises obtaining a recombinant antibody according to claim 1, and detecting interaction between the antibody and the one or more endogenous lipases or determining the binding affinity of the one or more endogenous lipases to the antibody.

33. (canceled)

34. The method of claim 32, wherein the interaction of lipase and antibody is detected by analysis of antibody produced in the host cell, for example by SPR, hydroxyl radical footprinting, native mass spectrometry (e.g., ESI-MS or SEC-MS), and/or ion mobility.

35. The method of claim 32, wherein the method further comprises determining binding affinity of lipase and antibody, for example by surface plasmon resonance (SPR), microscale thermophoresis (MST), and/or ELISA.

36. The method of claim 32, wherein the modification results in a reduced level of interaction with the one or more endogenous lipases of the host cell, such as lysosomal phospholipase A2 (LPLA2), phospholipase B-like protein (PLBL2), thioesterase, palmitoyl protein thioesterase (PPT), phospholipase D3 (PLD3), or sphingomyelin phosphodiesterase (SP).

37. The method of claim 36, wherein the modification results in a reduced level of interaction with LPLA2 and/or PLBL2.

38. The method of claim 32, wherein said modification results in at least a 5-fold, 10-fold, 20-fold, 30-fold, 50-fold, or 100-fold reduction in binding affinity (i.e., increase in K_{D}) of said antibody to said one or more endogenous lipases expressed by said host cell, optionally wherein binding affinity is determined by surface plasmon resonance (SPR), microscale thermophoresis (MST), and/or ELISA; or wherein said modification results in at least a 5-fold, 10-fold, 20-fold, 30-fold, 50-fold, or 100-fold reduction in the level of interaction of said antibody to said one or more endogenous lipases, for example, as determined by binding dissociation as measured by mass spectroscopy (e.g., by VC50) or by the amount of antibody-lipase complexes detected by SEC-MS or atmospheric ion mobility (e.g., IM-MS).

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. The method of claim 32, wherein the host cell is a Chinese hamster ovary (CHO) cell.

61. The method of claim 32, wherein the host cell is modified to: mutate, down-regulate, or knock-out one or both of alpha-Man-1 or alpha-Man-2; to inhibit processing of Asn-linked Man₉GlcNac₂ glycan precursors and/or to increase high molecular weight mannose species, such as Man6 or higher, Man7 or higher, or Man7-9, relative to Man3-5; and/or to increase expression of one or more enzymes that increase the chain length of glycans, such as GNT-1, GNT-2, GNT-3, GNT-4abc, GNT-5, or GalT.

62. A method of producing a recombinant protein or antibody, comprising:
a. expressing the protein or antibody in a host cell modified to: mutate, down-regulate, or knock-out one or both of alpha-Man-1 or alpha-Man-2; inhibit processing of Asn-linked Man₉GlcNac₂ glycan precursors and/or to increase high molecular weight mannose species, such as Man6 or higher, Man7 or higher, or Man7-9, relative to Man3-5; and/or increase expression of one or more enzymes that increase the chain length of glycans, such as GNT-1, GNT-2, GNT-3, GNT-4abc, GNT-5, or GalT; and
b. determining whether the protein or antibody has reduced interaction with one or more endogenous lipases expressed by said host cell as compared to a protein or antibody expressed from an unmodified host cell.

63. A method of producing a recombinant protein or antibody, comprising:
a. expressing the protein or antibody in a host cell under conditions that significantly increase the concentration of high molecular weight mannose species, such as Man6 or higher, Man7 or higher, or Man7-9 relative to overall mannosylated species, and
b. determining whether the protein or antibody has reduced interaction with one or more endogenous lipases expressed by said host cell as compared to a protein or antibody expressed from an unmodified host cell.

64. The method of claim 63, wherein the conditions comprise increasing the osmolality of the culture medium, for example, by at least 100 or at least 200 mOsm/kg, adding manganese chloride or ammonium chloride to the medium, increasing or adding raffinose, monensin, mannose, galactose, fructose, and/or maltose, or adding a high mannose promoting inhibitors such as Kifunensine to the medium.

65. A method of producing a recombinant protein or antibody, comprising:
a. expressing the protein or antibody in a host cell modified to eliminate at least one glycosylation site on at least one endogenous lipase; and
b. determining whether the protein or antibody has reduced interaction with one or more endogenous lipases expressed by said host cell as compared to a protein or antibody expressed from an unmodified host cell.

66. The method of claim 65, wherein the host cell is modified to eliminate at least one glycosylation site on LPLA2 and/or PLBL2.

67. The method of claim 65, wherein the modification comprises at least one amino acid substitution within at least one N-X-S/T site in at least one lipase enzyme of the host cell.

68. The method of claim 62, wherein the host cell is a CHO cell.

69. The method of claim 67, wherein the host cell is a CHO cell and wherein the modification comprises:
a. one or more amino acid substitutions in LPLA2 (SEQ ID NO: 2) at one or more of positions 39-41, 99-101, 273-275, and 289-291,
b. one or more amino acid substitutions in LPLA2 (SEQ ID NO: 2) at one or more of positions 125-131, 133-145, 146-177, 229-247, and 248-260
c. one or more amino acid substitutions in LPLA2 (SEQ ID NO: 2) at one or more of positions 146-177,
d. one or more amino acid substitutions in PLBL2 (SEQ ID NO: 3) at one or more of positions 47, 65, 69, 190, 395, and 474,
e. one or more amino acid substitutions in PLBL2 (SEQ ID NO: 3) at one or more of positions 67-78, 79-98, 173-187, 359-371, 372-388, 389-400, 401-407, 424-459, 211-236, 241-253, 287-333, 340-352, 513-530, 539-546, 573-599, 56-64, 485-512, and 548-572,
f. one or more amino acid substitutions in PLBL2 (SEQ ID NO: 3) at one or more of positions 79-98, 424-459, 573-599, 372-388, and 548-572,
g. one or more amino acid substitutions in thioesterase (SEQ ID NO: 1) at one or more of positions 298-300 and 422-424,
h. one or more amino acid substitutions in PPT (SEQ ID NO: 4) at one or more of positions 197-199, 212-214, and 232-234,
i. one or more amino acid substitutions in PLD3 (SEQ ID NO: 5) at one or more of positions 97-99, 102-104, 132-134, 234-236, 282-284, 385-387, and 430-432, and/or
j. one or more amino acid substitutions in SP (SEQ ID NO: 6) at one or more of positions 84-86, 173-175, 333-335, 393-395, 518-520, and 611-613.

70. The method of claim 62, wherein determining whether the protein or antibody has reduced interaction with one or more endogenous lipases is by SPR, hydroxyl radical footprinting, native mass spectrometry (e.g., ESI-MS or SEC-MS), and/or ion mobility analysis of the protein or antibody expressed from the host cell (e.g., IM-MS) as compared to the protein or antibody expressed from an unmodified host cell.
